SEQUENCE LISTING (1 sequence)

SEQ ID NO: 1 - protein, 20 aa, synthetic construct
RHKDESTNQCGPAVILMFYW

(19) United States
(12) Patent Application Publication
(10) Pub. No.: US 2025/0298016 A1
(43) Pub. Date: Sep. 25, 2025

(54) PLASMONIC PARTICLE SYSTEMS FOR SINGLE-ANALYTE ASSAYS

(71) Applicant: Nautilus Subsidiary, Inc., Seattle, WA (US)

(72) Inventors: Meysam Rezaei BARMI, Decatur, GA (US); Michael Augusto DARCY, Fremont, CA (US); Rukshan PERERA, Newark, CA (US)

(21) Appl. No.: 19/084,461

(22) Filed: Mar. 19, 2025

Related U.S. Application Data
(60) Provisional application No. 63/568,945, filed on Mar. 22, 2024.

(51) Int. Cl. G01N 33/553; G01N 33/53; G01N 33/543

(52) U.S. Cl. CPC G01N 33/553; G01N 33/5308; G01N 33/54346; G01N 33/54386

(57) ABSTRACT

Array-based systems, including single-molecule systems, for the interrogation of analytes are provided, in which the system contain metal nanoparticles that produce plasmonic interactions with incident light. Methods of utilizing plasmonic interactions between light fields and metal nanoparticles are provided during array-based characterization of analytes, including spectroscopic characterizations and affinity-based characterizations.

### CROSS-REFERENCE TO RELATED APPLICATIONS

This application claims priority to U.S. Provisional Application No. 63/568,945 filed on Mar. 22, 2024, which is incorporated herein by reference in its entirety.

### SEQUENCE LISTING

The instant application contains a Sequence Listing which has been submitted electronically in XML file format and is hereby incorporated by reference in its entirety. Said XML copy, created on Feb. 25, 2025, is named SL_50109.4024WO.xml and is 4,096 bytes in size.

### BACKGROUND

Certain optical effects can be enhanced by the presence of a material and a nanostructured surface. Analytical techniques such as surface-enhanced Raman spectroscopy and surface-enhanced infrared spectroscopy utilize contacting of molecules to nanostructures to enhance spectroscopic signals associated with the molecules. Nanoparticles may also be utilized to produce surface-enhanced fluorescence effects when fluorescent molecules are contacted to the nanoparticles in the presence of fluorescence-stimulating photons. In many cases, nanoparticles of noble metals may be useful for surface-enhanced optical techniques.

The assay of macromolecules, including biomolecules (e.g., polypeptides, nucleic acids) may be performed in array-based formats. Arrays provide an advantage of spatially separating molecules, thereby facilitating interrogation of individual molecules at single-analyte resolution. Array-based analytical methods may be useful for sequencing and/or characterizing macromolecules. Optical interrogation of molecules provided on arrays may utilize labeled (e.g., fluorescent or luminescent labels) techniques, or non-labeled techniques.

### SUMMARY

In an aspect, provided herein is a composition, comprising: a) a nucleic acid nanoparticle comprising a face, wherein the face contains a first attachment site and a second attachment site, b) a first metal nanoparticle and a second metal nanoparticle, wherein the first metal nanoparticle is attached to the first attachment site and the second metal nanoparticle is attached to the second attachment site, c) an entity coupled to the nucleic acid nanoparticle, wherein the entity is disposed between the first metal nanoparticle and the second metal nanoparticle, and d) a pendant single-stranded nucleic acid attached to the nucleic acid nanoparticle.

In another aspect, provided herein is a composition, comprising: a) a solid support, b) a nucleic acid nanoparticle attached to the solid support, wherein the nucleic acid nanoparticle comprises a face, wherein the face is substantially distal to the solid support, and wherein the face comprises a first attachment site, a second attachment site, and a third attachment site, c) first metal nanoparticle and a second metal nanoparticle, wherein the first metal nanoparticle is attached to the first attachment site and the second metal nanoparticle is attached to the second attachment site, and d) a polymeric chain coupled to the third attachment site, wherein the polymeric chain is disposed between the first metal nanoparticle and the second metal nanoparticle.

In another aspect, provided herein is a composition, comprising: a) an affinity agent, b) a linking moiety attached to the affinity agent, and c) a nanoparticle cluster attached to the linking moiety, wherein the nanoparticle cluster comprises a first metal nanoparticle, a second metal nanoparticle, and a fluorescent dye disposed between the first metal nanoparticle and the second metal nanoparticle.

In another aspect, provided herein is a method, comprising: a) coupling a binding reagent to an analyte, wherein the analyte is immobilized on a solid support, and wherein the binding reagent comprises a nanoparticle cluster, wherein the nanoparticle cluster comprises a first metal nanoparticle, a second metal nanoparticle, and a fluorescent dye disposed between the first metal nanoparticle and the second metal nanoparticle, b) contacting the nanoparticle cluster with light, and c) detecting a fluorescent signal from the fluorescent dye, thereby identifying an address of the solid support containing the binding reagent coupled to the analyte.

In another aspect, provided herein is a method, comprising: a) providing an analyte immobilized on a solid support, wherein the analyte is disposed on the solid support between a first metal nanoparticle and a second metal nanoparticle, b) contacting the analyte immobilized on the solid support with light, c) detecting scattering of the light contacted to the analyte immobilized on the solid support, and d) based upon the scattering of the light contacted to the analyte, identifying a structure of the analyte immobilized on the solid support.

In another aspect, provided herein is an array, comprising: a) a solid support comprising a plurality of optically resolvable sites, b) at each individual site of the plurality of optically resolvable sites, one and only one analyte coupled to each individual site, and c) binding reagents coupled to analytes at sites of the plurality of optically resolvable sites, wherein each individual binding reagent comprises no more than 5 detectable labels.

In another aspect, provided herein is a method, comprising: a) providing an array as set forth herein, b) illuminating the plurality of optically resolvable sites with a light field, and c) detecting at each individual site of the sites of the plurality of optically resolvable sites a detectable signal from a binding reagent of the binding reagents.

In another aspect, provided herein is a method, comprising: a) providing an analyte immobilized on a solid support at a fixed address, b) coupling a binding reagent to the analyte at the fixed address, wherein the binding reagent comprises an affinity agent coupled to a metal nanoparticle, c) detecting a detectable signal from the binding reagent at the fixed address, and d) after detecting the detectable signal, contacting the metal nanoparticle with a light field comprising light with an infrared wavelength.

### INCORPORATION BY REFERENCE

All publications, items of information available on the internet, patents, and patent applications cited in this specification are herein incorporated by reference to the same extent as if each individual publication, patent, or patent application was specifically and individually indicated to be incorporated by reference. To the extent publications, items of information available on the internet, patents, or patent applications incorporated by reference contradict the disclosure contained in the specification, the specification is intended to supersede and/or take precedence over any such contradictory material.

### BRIEF DESCRIPTION OF DRAWINGS

FIGS. 1A, 1B, 1C, 1D, 1E, 1F, 1G, 1H, and 1I depict various compositions containing plasmonic particle system, in accordance with some embodiments.

FIG. 2 illustrates various length scales associated with plasmonic particle system, in accordance with some embodiments.

FIGS. 3A, 3B, and 3C display steps of a method of detecting an analyte utilizing a binding reagent containing a plasmonic particle system, in accordance with some embodiments.

FIGS. 4A, 4B, 4C, and 4D show steps of a method of characterizing an analyte or macromolecule that is attached to a field-orientable nanoparticle, in accordance with some embodiments. FIG. 4E depicts an alternative configuration of the plasmonic particle system depicted in FIGS. 4A-4D.

FIGS. 5A, 5B, 5C, 5D, 5E, and 5F depict linking moiety compositions for attaching plasmonic particle systems to other object, in accordance with some embodiments.

FIGS. 6A and 6B illustrate a method of characterizing an analyte comprising a polymeric chain (SEQ ID NO: 1) by translation of the polymeric chain through a plasmonic particle system, in accordance with some embodiments.

FIG. 7 displays a schematic flow chart of a method of determining an optical signature for an analyte or macromolecule, in accordance with some embodiments.

FIGS. 8A, 8B, 8C, and 8D show steps of a method of dissociating a binding reagent utilizing irradiation of a metal nanoparticle, in accordance with some embodiments.

FIGS. 9A, 9B, 9C, and 9D depict methods of releasing array-bound entities from an array site by thermoplasmonic heating of metal nanoparticles, in accordance with some embodiments.

### DETAILED DESCRIPTION

It may be useful to provide plasmonic particle systems to single-analyte arrays for certain optical interrogation techniques. The plasmonic particle systems may comprise clusters of metallic nanoparticles that facilitate enhancement of optical signals associated with array entities. In some cases, a plasmonic particle system may be attached to a solid support of an array, thereby affixing the plasmonic particle system to a fixed address of the array. In other cases, a plasmonic particle system may be attached to a mobile array entity, such as an affinity agent that is configured to form binding interactions with macromolecules attached to an array.

Plasmonic particle systems may comprise clusters of nanoparticles that are coupled together. The nanoparticle clusters may comprise 2 or more individual nanoparticles (e.g., about 2, 3, 4, 5, 6, 7, 8, 9, or 10 individual nanoparticles). The nanoparticles may be clustered in close proximity and an array entity (e.g., a macromolecule, a detectable label) may be disposed between the nanoparticles of a nanoparticle cluster. Preferably, the array entity may be contacted to each of the nanoparticles of a nanoparticle cluster. Alternatively, an array entity may be disposed adjacent to one or more nanoparticles of a nanoparticle cluster.

Array compositions containing plasmonic particle systems, as set forth herein, may be associated with a sensing device that detects optical signals associated with array entities. The sensing device may be spatially resolved (e.g., a two-dimensional pixel array), thereby associating detected optical signals with specific array addresses. A sensed optical signal from a particular array address may be enhanced by the presence of a plasmonic particle system at the array address.

Provided herein are systems and methods that utilize plasmonic particle systems. Disclosed systems and methods can include systems and methods for detecting the binding of affinity reagents to analytes on analyte arrays utilizing plasmonic particle systems attached to the affinity reagents. Disclosed systems and methods can also include methods for characterizing analytes on analyte arrays utilizing plasmonic particle systems.

### Definitions

Terms used herein will be understood to take on their ordinary meaning in the relevant art unless specified otherwise. Several terms used herein and their meanings are set forth below.

As used herein, the term “plasmonic particle system” refers to a composition comprising two or more spatially separated metal nanoparticles and an entity disposed between the two or more spatially separated metal nanoparticles. Any two metal nanoparticles of a plasmonic particle system may be spatially separated by a separation gap as measured between respective nanoparticle centerpoints or between points of nearest approach of the respective surfaces of the nanoparticles. A plasmonic particle system may have a separation gap of at least about 0.1 nanometers (nm). An entity disposed between the metal nanoparticles of a plasmonic particle system may be disposed in the separation gap between the metal nanoparticles. An entity may be contacted to at least one metal nanoparticle, or optionally contacted to each metal nanoparticle of the plasmonic particle systems. Alternatively, an entity may not be contacted to any metal nanoparticle of a plasmonic particle system. Entities disposed between the metal nanoparticles can include small molecules (e.g., molecules having a molecular weight less than 1 kiloDalton), macromolecules (e.g., molecules having a molecular weight of at least 1 kiloDalton), analytes (e.g., polymeric molecules, biopolymers), and detectable labels (e.g., fluorescent molecules, luminescent molecules). A plasmonic particle system can further comprise one or more linking moieties. A linking moiety may couple a first metal nanoparticle to a second metal nanoparticle, or may couple an entity to a metal nanoparticle, or may couple the plasmonic particle system to another object (e.g., an array site, a particle, an affinity agent). As used herein, the term “nanoparticle cluster” refers to two or more metal nanoparticles that are coupled together. A nanoparticle cluster may be formed during the formation of a plasmonic particle system before an entity has been provided to the plasmonic particle system. A nanoparticle cluster may be characterized by a separation gap between metal nanoparticles as set forth herein for plasmonic particle systems.

As used herein, the term “binding reagent” refers to a composition comprising an affinity agent coupled to a detectable label. A binding reagent may comprise two or more affinity agents. A binding reagent may comprise two or more detectable labels. Optionally, a binding reagent may comprise a plasmonic particle system, as set forth herein. A binding reagent may comprise a particle (e.g., a nucleic acid nanoparticle, a polymer nanoparticle, a branched or dendrimeric nanoparticle) that couples one or more affinity agents to one or more detectable labels.

As used herein, the term “address” refers to a location in an array where a particular analyte (e.g. protein, peptide or unique identifier label) is present. An address can contain a single analyte, or it can contain a population of several analytes of the same species (i.e. an ensemble of the analytes). Alternatively, an address can include a population of different analytes. Addresses are typically discrete. The discrete addresses can be contiguous, or they can be separated by interstitial spaces. An array useful herein can have, for example, addresses that are separated by less than 100 microns, 10 microns, 1 micron, 100 nm, 10 nm or less. Alternatively or additionally, an array can have addresses that are separated by at least 10 nm, 100 nm, 1 micron, 10 microns, or 100 microns. The addresses can each have an area of less than 1 square millimeter, 500 square microns, 100 square microns, 10 square microns, 1 square micron, 100 square nm or less. An array can include at least about 1x104, 1x105, 1x106, 1x107, 1x108, 1x109, 1x1010, 1x1011, 1x1012, or more addresses.

As used herein, the term “affinity agent” refers to a molecule or other substance that is capable of specifically or reproducibly binding to an analyte (e.g. protein). An affinity reagent can be larger than, smaller than or the same size as the analyte. An affinity reagent may form a reversible or irreversible bond with an analyte. An affinity reagent may bind with an analyte in a covalent or non-covalent manner. Affinity reagents may include reactive affinity reagents, catalytic affinity reagents (e.g., kinases, proteases, etc.) or non-reactive affinity reagents (e.g., antibodies or fragments thereof). An affinity reagent can be non-reactive and non-catalytic, thereby not permanently altering the chemical structure of an analyte to which it binds. Affinity reagents that can be particularly useful for binding to proteins include, but are not limited to, antibodies or functional fragments thereof (e.g., Fab′ fragments, F(ab′)2 fragments, single-chain variable fragments (scFv), di-scFv, tri-scFv, or microantibodies), affibodies, affilins, affimers, affitins, alphabodies, anticalins, avimers, DARPins, monobodies, nanoCLAMPs, nucleic acid aptamers, protein aptamers, lectins or functional fragments thereof.

As used herein, the terms “analyte” and “analyte of interest,” when used in reference to a structured nucleic acid particle, refer to a molecule, particle, or complex of molecules or particles that is coupled to a display moiety of a structured nucleic acid particle. An analyte may comprise a target for an analytical method (e.g., sequencing, identification, quantification, etc.) or may comprise a functional element such as a binding ligand or a catalyst. An analyte may comprise a biomolecule, such as a polypeptide, polysaccharide, nucleic acid, lipid, metabolite, enzyme cofactor or a combination thereof. An analyte may comprise a non-biological molecule, such as a polymer, metal, metal oxide, ceramic, semiconductor, mineral, or a combination thereof. As used herein, the terms “sample analyte” refers to an analyte derived from a sample collected from a biological or non-biological system. A sample analyte may be purified or unpurified. As used herein, the term “control analyte” refers to an analyte that is provided as a positive or negative control for comparison to a sample analyte. A control analyte may be derived from the same source as a sample analyte, or derived from a differing source from the sample analyte. As used herein, the term “standard analyte” refers to a known or characterized analyte that is provided as a physical or chemical reference to a process. A standard analyte may comprise the same type of analyte as a sample analyte, or may differ from a sample analyte. For example, a polypeptide analyte process may utilize a polypeptide standard analyte with known characteristics. In another example, a polypeptide analyte process may utilize a non-polypeptide standard analyte with known characteristics. As used herein, the term “inert analyte” refers to an analyte with no expected function in a process or system.

As used herein, the term “array” refers to a population of analytes (e.g. proteins) that are associated with unique identifiers such that the analytes can be distinguished from each other. A unique identifier can be, for example, a solid support (e.g. particle or bead), address on a solid support, tag, label (e.g. luminophore), or barcode (e.g. nucleic acid barcode) that is associated with an analyte and that is distinct from other identifiers in the array. Analytes can be associated with unique identifiers by attachment, for example, via covalent bonds or non-covalent bonds (e.g. ionic bond, hydrogen bond, van der Waals forces, electrostatics etc.). An array can include different analytes that are each attached to different unique identifiers. An array can include different unique identifiers that are attached to the same or similar analytes. An array can include separate solid supports or separate addresses that each bear a different analyte, wherein the different analytes can be identified according to the locations of the solid supports or addresses.

As used herein, the term “attached” refers to the state of two things being joined, fastened, adhered, connected or bound to each other. Attachment can be covalent or non-covalent. For example, a particle can be attached to a protein by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions, adhesion, adsorption, and hydrophobic interactions.

As used herein, the term “binding profile” refers to a plurality of binding outcomes for a protein or other analyte. The binding outcomes can be obtained from independent binding observations, for example, independent binding outcomes can be acquired using different affinity reagents, respectively. Alternatively, the binding outcomes can be generated in silico, for example, being derived from a modification of an empirically obtained binding outcome. A binding profile can include empirical measurement outcomes, candidate measurement outcomes, calculated measurement outcomes, theoretical measurement outcomes or a combination thereof. A binding profile can exclude one or more of empirical measurement outcomes, candidate measurement outcomes, calculated measurement outcomes, or theoretical measurement outcomes or putative measurement outcomes. A binding profile can include a vector of binding outcomes.

As used herein, the term “conformational state,” when used in reference to a molecule or particle, refers to the shape or proportionate dimensions of the molecule or particle. At the molecular level conformational state can be characterized by the spatial arrangement of a molecule that results from the rotation of its atoms about their bonds. The conformational state of a macromolecule, such as a protein or nucleic acid, can be characterized in terms of secondary structure, tertiary structure, or quaternary structure. Secondary structure of a nucleic acid is the set of interactions between bases of the nucleic acid such as interactions formed by internal complementarity in a single stranded nucleic acid or by complementarity between two strands in a double helix. Tertiary structure of a nucleic acid is the three-dimensional shape of the nucleic acid as defined, for example, by the relative locations of its atoms in three-dimensional space. Quaternary structure of a nucleic acid is the overall shape resulting from interactions between two or more nucleic acids at a higher level than the secondary or tertiary levels. Secondary structure of a protein is the three-dimensional form of local segments of the protein which can be defined, for example, by the pattern of hydrogen bonds between the amino hydrogen and carboxyl oxygen atoms in the peptide backbone or by the regular pattern of backbone dihedral angles in a particular region of the Ramachandran plot for the protein. Tertiary structure of a protein is the three-dimensional shape of a single polypeptide chain backbone including, for example, interactions and bonds of side chains that form domains. Quaternary structure of a protein is the three-dimensional shape and interaction between the amino acids of multiple polypeptide chain backbones. A molecule or particle having a given composition may take on more than one conformational state with or without changes to its composition. For example, a protein having a given amino acid sequence (i.e. protein primary structure) may take on different conformations at the secondary, tertiary or quaternary level, and a nucleic acid having a given nucleotide sequence (i.e. nucleic acid primary structure) may take on different conformations at the secondary, tertiary or quaternary level.

The term “comprising” is intended herein to be open-ended, including not only the recited elements, but further encompassing any additional elements.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “face” refers to a portion of a molecule, particle, or complex (e.g., a SNAP or a SNAP complex) that contains one or more moieties with substantially similar orientation and/or function. For example, a substantially rectangular or square SNAP may have a coupling face that comprises one or more coupling moieties, with each coupling moiety having a substantially similar orientation to each other coupling moiety (e.g., oriented about 1800 from a display moiety that is configured to be coupled to an analyte). In another example, a spherical nanoparticle may have a coupling face comprising a coupled plurality of coupling moieties confined to a hemisphere of the particle (i.e., a plurality of coupling moieties having similar function but differing orientations). In some cases, a face may be defined by an imaginary plane relative to which a moiety or a portion thereof may have a spatial proximity or angular orientation when the plane is contacted with a point or portion of a molecule, particle, or complex. A moiety or a portion thereof may have a spatial separation from an imaginary plane defining a face of a molecule, particle, or complex of no more than about 100 nanometers (nm), 90 nm, 80 nm, 70 nm, 60 nm, 50 nm, 40 nm, 30 nm, 25 nm 20 nm, 15 nm, 10 nm, 9 nm, 8 nm, 7 nm, 6 nm, 5 nm, 4 nm, 3 nm, 2 nm, 1 nm, 0.5 nm, 0.1 nm, or less than 0.1 nm. A moiety or a portion thereof may have an angular orientation relative to a normal vector of an imaginary plane of no more than about 90°, 85°, 80°, 75°, 70°, 65°, 60°, 55°, 50°, 45°, 40°, 35°, 30°, 25°, 20°, 15°, 10°, 5°, 1°, or less than 1°.

As used herein, the terms “group” and “moiety” are intended to be synonymous when used in reference to the structure of a molecule. The terms refer to a component or part of the molecule. The terms do not necessarily denote the relative size of the component or part compared to the rest of the molecule, unless indicated otherwise.

As used herein, the term “immobilized,” when used in reference to a molecule that is in contact with a fluid phase, refers to the molecule being prevented from diffusing in the fluid phase. For example, immobilization can occur due to the molecule being confined at, or attached to, a solid phase. Immobilization can be temporary (e.g. for the duration of one or more steps of a method set forth herein) or permanent. Immobilization can be reversible or irreversible under conditions utilized for a method, system or composition set forth herein.

As used herein, the terms “label” and “detectable label” refer synonymously to a molecule or moiety that provides a detectable characteristic. The detectable characteristic can be, for example, an optical signal such as absorbance of radiation, luminescence emission, luminescence lifetime, luminescence polarization, fluorescence emission, fluorescence lifetime, fluorescence polarization, or the like; Rayleigh and/or Mie scattering; binding affinity for a ligand or receptor; magnetic properties; electrical properties; charge; mass; radioactivity or the like. Exemplary labels include, without limitation, a fluorophore, luminophore, chromophore, nanoparticle (e.g., gold, silver, carbon nanotubes), heavy atoms, radioactive isotope, mass label, charge label, spin label, receptor, ligand, or the like. A label may produce a signal that is detectable in real-time (e.g., fluorescence, luminescence, radioactivity). A label may produce a signal that is detected off-line (e.g., a nucleic acid barcode) or in a time-resolved manner (e.g., time-resolved fluorescence). A label may produce a signal with a characteristic frequency, intensity, polarity, duration, wavelength, sequence, or fingerprint.

As used herein, the terms “linker” and “linking moiety” refer synonymously to a moiety that connects two objects to each other. One or both objects can be a molecule, solid support, address, particle or bead. Both objects can be moieties of a molecule, solid support, address, particle or bead. The term can also refer to an atom, moiety or molecule that is configured to react with two objects to form a moiety that connects the two objects. The connection of a linker to one or both objects can be a covalent bond or non-covalent bond. A linker may be configured to provide a chemical or mechanical property to the moiety connecting two objects, such as hydrophobicity, hydrophilicity, electrical charge, polarity, rigidity, or flexibility. A linker may comprise two or more functional groups that facilitate coupling of the linker to the first and second objects. A linker may include a polyfunctional linker such as a homobifunctional linker, heterobifunctional linker, homopolyfunctional linker, or heteropolyfunctional linker. Exemplary compositions for linkers can include, but are not limited to, a polyethylene glycol (PEG), polyethylene oxide (PEO), amino acid, protein, nucleotide, nucleic acid, nucleic acid origami, dendrimer, protein nucleic acid (PNA), polysaccharide, carbon, nitrogen, oxygen, ether, sulfur, or disulfide. A linker can be a bead or particle such as a structured nucleic acid particle.

As used herein, the term “nucleic acid nanoparticle” refers to a single- or multi-chain polynucleotide molecule comprising a compacted three-dimensional structure. The compacted three-dimensional structure can optionally have a characteristic tertiary structure. An exemplary nucleic acid nanostructure is a structured nucleic acid particle (SNAP). A SNAP can be configured to have an increased number of interactions between regions of a polynucleotide strand, less distance between the regions, increased number of bends in the strand, and/or more acute bends in the strand, as compared to the same nucleic acid molecule in a random coil or other non-structured state. Alternatively or additionally, the compacted three-dimensional structure of a nucleic acid nanostructure can optionally have a characteristic quaternary structure. For example, a nucleic acid nanostructure can be configured to have an increased number of interactions between polynucleotide strands or less distance between the strands, as compared to the same nucleic acid molecule in a random coil or other non-structured state. In some configurations, the tertiary structure (i.e. the helical twist or direction of the polynucleotide strand) of a nucleic acid nanostructure can be configured to be more dense than the same nucleic acid molecule in a random coil or other non-structured state. Nucleic acid nanostructures may include deoxyribonucleic acid (DNA), ribonucleic acid (RNA), peptide nucleic acid (PNA), other nucleic acid analogs, and combinations thereof. Nucleic acid nanostructures may have naturally-arising or engineered secondary, tertiary, or quaternary structures. A structured nucleic acid particle can contain at least one of: i) a moiety that is configured to couple an analyte to the nucleic acid nanostructure, ii) a moiety that is configured to couple the nucleic acid nanostructure to another object such as another SNAP, a solid support or a surface thereof, iii) a moiety that is configured to provide a chemical or physical property or characteristic to a nucleic acid nanostructure, or iv) a combination thereof. Exemplary SNAPs may include nucleic acid nanoballs (e.g. DNA nanoballs), nucleic acid nanotubes (e.g. DNA nanotubes), and nucleic acid origami (e.g. DNA origami). A SNAP may be functionalized to include one or more reactive handles or other moieties. A SNAP may comprise one or more incorporated residues that contain reactive handles or other moieties (e.g., modified nucleotides).

As used herein, the term “nucleic acid origami” refers to a nucleic acid construct having an engineered tertiary or quaternary structure. A nucleic acid origami may include DNA, RNA, PNA, modified or non-natural nucleic acids, or combinations thereof. A nucleic acid origami may include a plurality of oligonucleotides that hybridize via sequence complementarity to produce the engineered structuring of the origami. A nucleic acid origami may include sections of single-stranded or double-stranded nucleic acid, or combinations thereof. Exemplary nucleic acid origami structures may include nanotubes, nanowires, cages, tiles, nanospheres, blocks, and combinations thereof. A nucleic acid origami can optionally include a relatively long scaffold nucleic acid to which multiple smaller nucleic acids hybridize, thereby creating folds and bends in the scaffold that produce an engineered structure. The scaffold nucleic acid can be circular or linear. The scaffold nucleic acid can be single stranded but for hybridization to the smaller nucleic acids. A smaller nucleic acid (sometimes referred to as a “staple”) can hybridize to two regions of the scaffold, wherein the two regions of the scaffold are separated by an intervening region that does not hybridize to the smaller nucleic acid.

As used herein, the terms “protein” and “polypeptide” refer synonymously to a molecule comprising two or more amino acids joined by a peptide bond. A protein may also be referred to as a polypeptide, oligopeptide or peptide. A protein can be a naturally-occurring molecule, or synthetic molecule. A protein may include one or more non-natural amino acids, modified amino acids, or non-amino acid linkers. A protein may contain D-amino acid enantiomers, L-amino acid enantiomers or both. Amino acids of a protein may be modified naturally or synthetically, such as by post-translational modifications. In some circumstances, different proteins may be distinguished from each other based on different genes from which they are expressed in an organism, different primary sequence length or different primary sequence composition. Proteins expressed from the same gene may nonetheless be different proteoforms, for example, being distinguished based on non-identical length, non-identical amino acid sequence or non-identical post-translational modifications. Different proteins can be distinguished based on one or both of gene of origin and proteoform state.

As used herein, the term “single,” when used in reference to an object such as an analyte, means that the object is individually manipulated or distinguished from other objects. A single analyte can be a single molecule (e.g. single protein), a single complex of two or more molecules (e.g. a multimeric protein having two or more separable subunits, a single protein attached to a structured nucleic acid particle or a single protein attached to an affinity reagent), a single particle, or the like. Reference herein to a “single analyte” in the context of a composition, system or method herein does not necessarily exclude application of the composition, system or method to multiple single analytes that are manipulated or distinguished individually, unless indicated contextually or explicitly to the contrary.

As used herein, the term “solid support” refers to a substrate that is insoluble in aqueous liquid. Optionally, the substrate can be rigid. The substrate can be non-porous or porous. The substrate can optionally be capable of taking up a liquid (e.g. due to porosity) but will typically, but not necessarily, be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, inorganic glasses, optical fiber bundles, gels, and polymers. In particular configurations, a flow cell contains the solid support such that fluids introduced to the flow cell can interact with a surface of the solid support to which one or more components of a binding event (or other reaction) is attached.

As used herein, the term “structured nucleic acid particle” or “SNAP” refers to a single- or multi-chain polynucleotide molecule having a compacted three-dimensional structure. The compacted three-dimensional structure can optionally be characterized in terms of hydrodynamic radius or Stoke's radius of the SNAP relative to a random coil or other non-structured state for a nucleic acid having the same sequence length as the SNAP. The compacted three-dimensional structure can optionally be characterized with regard to tertiary structure. For example, a SNAP can be configured to have an increased number of internal binding interactions between regions of a polynucleotide strand, less distance between the regions, increased number of bends in the strand, and/or more acute bends in the strand, as compared to a nucleic acid molecule of similar length in a random coil or other non-structured state. Alternatively or additionally, the compacted three-dimensional structure can optionally be characterized with regard to tertiary or quaternary structure. For example, a SNAP can be configured to have an increased number of interactions between polynucleotide strands or less distance between the strands, as compared to a nucleic acid molecule of similar length in a random coil or other non-structured state. In some configurations, the secondary structure of a SNAP can be configured to be more dense than a nucleic acid molecule of similar length in a random coil or other non-structured state. A SNAP may contain DNA, RNA, PNA, modified or non-natural nucleic acids, or combinations thereof. A SNAP may include a plurality of oligonucleotides that hybridize to form the SNAP structure. The plurality of oligonucleotides in a SNAP may include oligonucleotides that are attached to other molecules (e.g., probes, analytes such as proteins, reactive moieties, or detectable labels) or are configured to be attached to other molecules (e.g., by functional groups). A SNAP may include engineered or rationally designed structures. Exemplary SNAPs include nucleic acid origami and nucleic acid nanoballs.

As used herein, the terms “type” and “species,” when used in reference to a subset of analytes, refer synonymously to a characteristic that is shared by the analytes in the subset and that distinguishes the analytes in the subset from analytes that are not in the subset. The characteristic can be any of a variety of characteristics known for the analytes. Any of a variety of analytes can be categorized by type, including for example, proteins. Exemplary characteristics that can be used to categorize proteins by type include, but are not limited to, amino acid composition, full length amino acid sequence, proteoform, presence or absence of an amino acid sequence motif, number of amino acids present (i.e. sequence length), molecular weight, presence or absence of a particular epitope, presence or absence of epitope(s) recognized by a particular affinity reagent, probability of binding a particular affinity reagent, presence or absence of a post-translational modification, enzymatic activity, affinity for binding a particular protein or protein motif, or the like.

The embodiments set forth below and recited in the claims can be understood in view of the above definitions.

### Plasmonic Particle Systems

Provided herein is a composition comprising: a) a nucleic acid nanoparticle comprising a face, in which the face contains a first attachment site and a second attachment site, b) a first metal nanoparticle and a second metal nanoparticle, in which the first metal nanoparticle is attached to the first attachment site and the second metal nanoparticle is attached to the second attachment site, c) an entity coupled to the nucleic acid nanoparticle, in which the entity is disposed between the first metal nanoparticle and the second metal nanoparticle, and d) a pendant single-stranded nucleic acid attached to the nucleic acid nanoparticle.

In another aspect, provided herein is a composition, comprising: a) a solid support, b) a nucleic acid nanoparticle attached to the solid support, wherein the nucleic acid nanoparticle comprises a face, in which the face is substantially distal to the solid support, and in which the face comprises a first attachment site, a second attachment site, and a third attachment site, c) first metal nanoparticle and a second metal nanoparticle, in which the first metal nanoparticle is attached to the first attachment site and the second metal nanoparticle is attached to the second attachment site, and d) a polymeric chain coupled to the third attachment site, in which the polymeric chain is disposed between the first metal nanoparticle and the second metal nanoparticle.

In another aspect, provided herein is a composition, comprising: a) an affinity agent, b) a linking moiety attached to the affinity agent, and c) a nanoparticle cluster attached to the linking moiety, in which the nanoparticle cluster comprises a first metal nanoparticle, a second metal nanoparticle, and a fluorescent dye disposed between the first metal nanoparticle and the second metal nanoparticle.

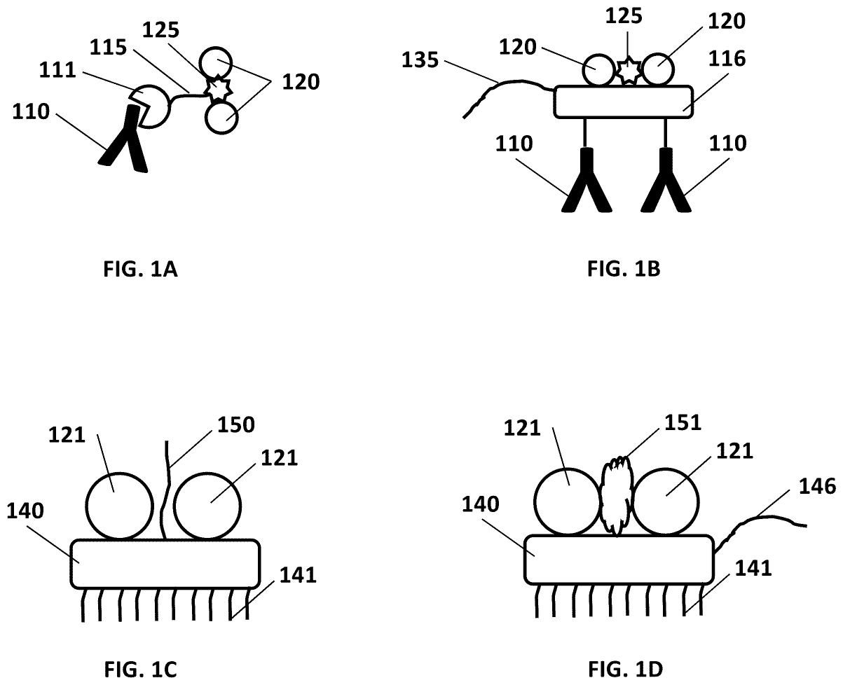

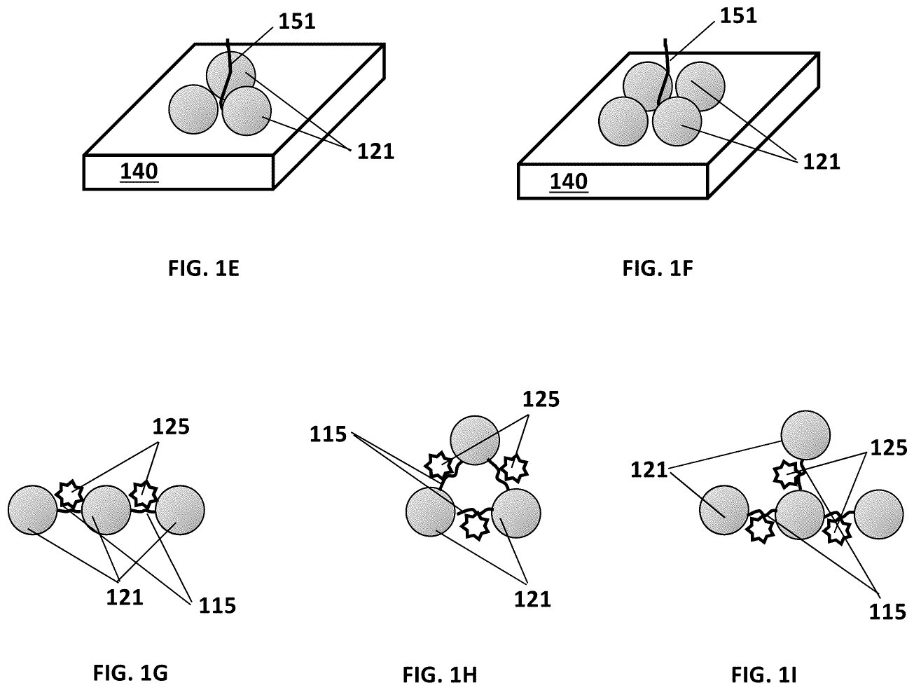

FIGS. 1A-1F illustrate aspects of compositions comprising plasmonic particle systems. FIGS. 1A-1B illustrate configurations of binding reagents containing affinity agents and plasmonic particle systems. FIG. 1A depicts a binding reagent comprising an affinity agent 110 coupled to a plasmonic particle system by a linking moiety 115. The linking moiety 115 may be covalently attached to the affinity agent 110. Alternatively, the linking moiety 115 may be non-covalently attached to the affinity agent 110. For example, FIG. 1A depicts an antibody affinity agent 110 that is attached to a linking moiety 115 by an immunoglobulin-binding protein 111 (e.g., protein A, protein G). The linking moiety 115 is further attached to a plasmonic particle system comprising at least two metal nanoparticles 120 and a detectable label 125 (e.g., a fluorescent dye, a luminescent dye) disposed between the metal nanoparticles 120. FIG. 1B depicts a binding reagent comprising a plurality of affinity agents 110 and a plasmonic particle system comprising at least two metal nanoparticles 120 and a detectable label 125. The affinity agents 110, metal nanoparticles 120, and detectable label 125 may be coupled together by a retaining moiety 116 (e.g., a nucleic acid nanoparticle). A binding reagent may further comprise a tether strand 135, as set forth herein, that is configured to increase the avidity of the binding reagent in the presence of a complementary docker strand (not shown). In some configurations, a tether strand 135 may comprise a pendant oligonucleotide. Optionally, the retaining moiety 116 may comprise a first face and a second face, in which an affinity agent is attached to the first face and a plasmonic particle system is attached to the second face. Preferably, the first face and the second face may be in a substantially opposed orientation or substantially distal from each other. Additional useful configurations and aspects of binding reagents are set forth in U.S. Pat. No. 11,692,217, which is herein incorporated by reference in its entirety.

FIGS. 1C-1D illustrate configurations of particles (e.g., nucleic acid nanoparticles) that may be useful for attaching analytes to sites on solid supports. FIG. 1C depicts a nanoparticle 140 (e.g., a nucleic acid nanoparticle) comprising a plurality of surface-coupling moieties 141 (e.g., pendant oligonucleotides). The surface-coupling moieties 141 may be configured to bind to complementary surface-coupled moieties of a site on a solid support (not shown). The nanoparticle 140 is further coupled to a plasmonic particle system comprising at least two metal nanoparticles 121 and an analyte 150 that is provided in a denatured or substantially unstructured configuration. FIG. 1D depicts an alternative configuration to the configuration of FIG. 1C, in which the analyte 151 is provided in a folded or structured configuration. Optionally, the retaining moiety 140 may comprise a first face and a second face, in which a surface-coupling moiety 141 is attached to the first face and a plasmonic particle system is attached to the second face. Preferably, the first face and the second face may be in a substantially opposed orientation or substantially distal from each other. Optionally, the nanoparticle 140 comprises a pendant docker strand 146, as set forth herein. The skilled person will readily recognize that additional metal nanoparticles 121 can be attached to a particle 140 in configurations that dispose an entity (e.g., a detectable label, an analyte) between each nanoparticle.

FIGS. 1E-1F illustrate particles comprising more than two metal nanoparticles. FIG. 1E depicts a particle 140 comprising a face that is attached to three metal nanoparticles 121 in a triangular configuration. An analyte 151 is disposed between the three metal nanoparticles 151. FIG. 1F depicts a particle 140 comprising a face that is attached to four metal nanoparticles 121 in a rectangular configuration. An analyte 151 is disposed between the four metal nanoparticles 151. The skilled person can readily provide particles with sufficient quantity of attachment sites to couple any necessary quantity of metal nanoparticles to the particle, such as about 2, 3, 4, 5, 6, 7, 8, 9, 10, or more than 10 particles.

FIGS. 1G-1I depict illustrate configurations of plasmonic particle system containing two or more metal nanoparticles, in which the metal nanoparticles are not attached to a particle. FIG. 1G depicts a linear or chain-like configuration, in which terminal metal nanoparticles 121 are attached to only one other metal nanoparticles 121, while medial metal nanoparticles 121 are attached to two other metal nanoparticles 121. The metal nanoparticles may be attached by one or more optional linking moieties 115. An entity (e.g., a detectable label 125 or an analyte or macromolecule) may be disposed between at least pair of adjacent metal nanoparticles 121. Optionally, an entity may be disposed between each pair of adjacent metal nanoparticles 121. FIG. 1H depicts a closed configuration, in which each metal nanoparticle 121 is attached to at least two other metal nanoparticles. FIG. 1I depicts a branched configuration, in which at least one metal nanoparticle 121 is attached to three or more metal nanoparticles 121. The skilled person will readily recognize numerous combinations and variations of the configurations depicted in FIGS. 1G-1I as additional nanoparticles are added to the nanoparticle cluster.

It may be useful to provide a plasmonic particle system on a particle with a controllable architecture, such as a nucleic acid nanoparticle. Preferably the particle can be provided with attachment sites at specific locations that facilitate the attachment of entities (e.g., an analyte, a detectable label, a metal nanoparticle) at the specific locations. Accordingly, a useful particle will facilitate the spatial arrangement and orientation of entities with respect to each other when the entities are attached to the particle. For example, a nucleic acid nanoparticle can comprise a face containing at least three attachment sites, in which two attachment sites are configured to bind a metal nanoparticle and a third attachment site is provided to bind an analyte or a detectable label between the two metal nanoparticles.

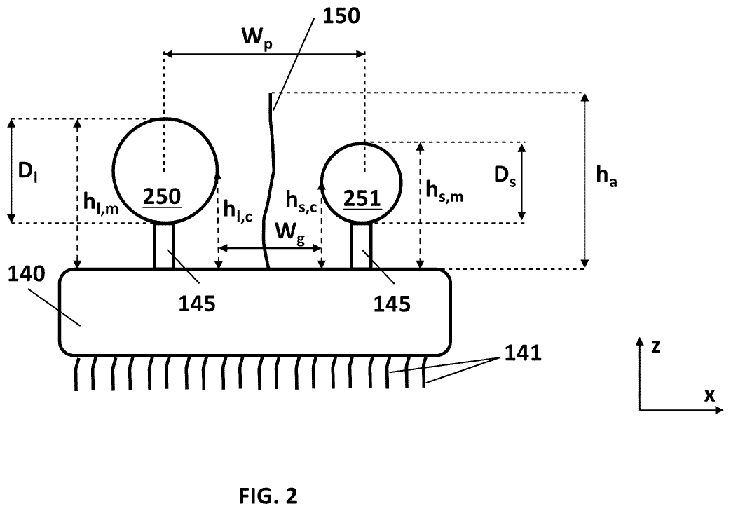

FIG. 2 depicts potentially useful characteristic dimensions of a particle comprising a plasmonic particle system. The composition comprises a particle 140 optionally comprising a plurality of surface-coupling moieties 141 on a first face. The particle 140 further comprises a second face that is substantially opposed to the first face. The second face comprises two attachment sites, in which the attachment sites optionally comprise spacing moieties 145 (e.g., a flexible linker, a rigid linker). The left attachment site is coupled to a larger metal nanoparticle 250 of diameter or height Dₗ, and the right attachment site is coupled to a smaller metal nanoparticle 251 of diameter or height Dₛ. An analyte 150 is attached to the particle 140 at a location between the larger metal nanoparticle 250 and the smaller metal nanoparticle 251. The separation distance between the larger metal nanoparticle 250 and the smaller metal nanoparticle 251 may be defined with respect to the centerpoints of the respective metal nanoparticles (wₚ) or with respect to the smallest distance between the outer surfaces of the two metal nanoparticles (w_{g}). If a metal nanoparticle is provided on a spacing moiety 145, the height of the metal nanoparticle may be defined as the height of the centerpoint of the metal nanoparticle (e.g., h_{l,c}, h_{s,c}) with respect to the second face of the particle 145 or may be defined as the maximum height of the metal nanoparticle (e.g., h_{l,m}, h_{s,m}) with respect to the second face of the particle 140. The height of the analyte 150, ha, may be characterized as the maximum distance of extent in the z-axis direction of the analyte 150. If the maximum distance of extent in the z-axis direction of the analyte 150 is temporally variable, ha may be determined or estimated as a temporal average of the maximum distance of extent in the z-axis direction of the analyte 150. Although FIG. 2 depicts a particle that is coupled to an analyte, a particle comprising a plasmonic particle system for a detectable binding reagent may be characterized by similar dimensions with respect to the location and orientation of the metal nanoparticles of the plasmonic particle system.

A plasmonic particle system may comprise two or more metal nanoparticles that are coupled together. A metal nanoparticle of a plasmonic particle system may comprise a noble metal. In some cases, a metal nanoparticle may comprise a metal selected from the group consisting of rhenium, ruthenium, rhodium, palladium, silver, osmium, iridium, platinum, gold, copper, and combinations or alloys thereof. A plasmonic particle system may comprise a first metal nanoparticle and a second metal nanoparticle, in which the first metal nanoparticle has a same atomic composition as the second metal nanoparticle. For example, a plasmonic particle system may comprise a first gold nanoparticle and a second gold nanoparticle. Alternatively, a plasmonic particle system may comprise a first metal nanoparticle and a second metal nanoparticle, in which an atomic composition of the first metal nanoparticle differs from an atomic composition of the second metal nanoparticle. For example, a plasmonic particle system may comprise a first gold nanoparticle and a second silver nanoparticle.

Useful aspects of plasmonic particle system, including aspects of plasmonic particle system associated with other particles (e.g., nucleic acid nanoparticles) may be described in Perez-Jimenez, A. I., et al. “Surface-enhanced Raman Spectrosocpy: Benefits, Trade-offs and Future Developments.” Chem. Sci. (2020); Mehmandoust, S., et al. “A Review of Fabrication of DNA Origami Plasmonic Structures for the Development of Surface-Enhanced Raman Scattering (SERS) Platforms.” Plasmonics (2023); Kanehira, Y., et al. “The Effect of Nanoparticle Composition on the Surface-Enhanced Raman Scattering Performance of Plasmonic DNA Origami Nanoantennas.” ACS Nano (2023); Simoncelli, S., et al. “Quantitative Single-Molecule Surface-Enhanced Raman Scattering by Optothermal Tuning of DNA Origami-Assembled Plasmonic Nanoantennas.” ACS Nano (2016); Tanwar, S., et al. “Broadband SERS Enhancement by DNA Origami Assembled Bimetallic Nanoantennas with Label-Free Single Proein Sensing.” J. Phys. Chem. Lett. (2021); and Kuhler, P., et al. “Plasmonic DNA-Origami Nanoantennas for Surface-Enhanced Raman Spectroscopy.” Nano. Lett. (2014), each of which is herein incorporated by reference in its entirety.

A metal nanoparticle of a plasmonic particle system may have a diameter of at least about 5 nanometers (nm), 10 nm, 20 nm, 30 nm, 40 nm, 50 nm, 60 nm, 70 nm, 80 nm, 90 nm, 100 nm, 120 nm, 150 nm, 200 nm, or more than 200 nm. Alternatively or additionally, a metal nanoparticle of a plasmonic particle system may have a diameter of no more than about 200 nm, 150 nm, 120 nm, 100 nm, 90 nm, 80 nm, 70 nm, 60 nm, 50 nm, 40 nm, 30 nm, 20 nm, 10 nm, 5 nm, or less than 5 nm. A plasmonic particle system may comprise a first metal nanoparticle and a second metal nanoparticle, in which a diameter of the first metal nanoparticle is substantially the same (e.g., within about ±1%, ±2%, ±3%, ±4%, ±5%, ±10%, ±15%,) as a diameter of the second metal nanoparticle. Alternatively, a plasmonic particle system may comprise a first metal nanoparticle and a second metal nanoparticle, in which a diameter of the first metal nanoparticle is substantially larger (e.g., ±20%, ±30%, ±40%, ±50%, ±75%, ±100%, ±200%) than a diameter of the second metal nanoparticle.

A plasmonic particle system may comprise a first metal nanoparticle and a second metal nanoparticle, in which the first metal nanoparticle can be spatially separated from the second metal nanoparticle by a separation gap (e.g., with respect to particle centerpoints, with respect to nearest points of approach of particle surfaces) of at least about 1 nanometer (nm), 2 nm, 3 nm, 4 nm, 5 nm, 10 nm, 15 nm, 20 nm, 30 nm, 40 nm, 50 nm, 100 nm, or more than 100 nm. Alternatively or additionally, the first metal nanoparticle can be spatially separated from the second metal nanoparticle by a separation gap of at no more than about 100 nanometer nm, 50 nm, 40 nm, 30 nm, 20 nm, 15 nm, 10 nm, 5 nm, 4 nm, 3 nm, 2 nm, 1 nm, or less than 1 nm. A separation gap between a first metal nanoparticle and a second metal nanoparticle may depend upon a dimension (e.g., length, width) of an entity that is to be disposed between the two metal nanoparticles. For example, a detectable label (e.g., a fluorescent or luminescent molecule) disposed between two metal nanoparticles may have a smaller separation gap (e.g., no more than 5 nm, 4 nm, 3 nm, 2 nm, 1 nm, or less than 1 nm) due to the dimensions of the detectable label. In another example, a polymeric molecule (e.g., a polypeptide, a polynucleotide, a polysaccharide, etc.) disposed between two metal nanoparticles may have a larger separation gap (e.g., at least about 10 nm, 15 nm, 20 nm, 30 nm, 40 nm, 50 nm, 100 nm, or more than 100 nm) if the polymeric molecule is provided in a compacted, folded, or globular form.

In some cases, a plasmonic particle system may be coupled to a particle (e.g., a nucleic acid nanoparticle, a branched or dendrimeric polymer particle). A particle may comprise at least two attachment sites, in which each individual attachment site of the at least two attachment sites is configured to bind to a single metal nanoparticle. A metal nanoparticle may be covalently or non-covalently attached to an attachment site of a particle. In some cases, a metal nanoparticle may be functionalized with one or more functional groups on its surface, in which the one or more functional groups facilitate attachment of the metal nanoparticle to an attachment site. For example, a reactive functional group on a surface of a metal nanoparticle may be covalently attached to a complementary reactive functional group of an attachment site, or a reactive functional group on a surface of a metal nanoparticle may be covalently attached to a complementary reactive functional group of an attachment site by a cross-linking molecule, or a coupling moiety may be attached to a functional group of the metal nanoparticle, thereby facilitating attachment of the coupling moiety to a complementary coupling moiety of the attachment site (e.g., complementary nucleic acids, a receptor-ligand binding pair). Additional aspects of nucleic acid nanoparticles are set forth in U.S. Pat. Nos. 11,203,612 and 11,505,796, each of which is herein incorporated by reference in its entirety.

In some cases, a particle having an attached plasmonic particle system may further comprise a third attachment site, in which an entity is attached to the third attachment site. The third attachment site may be spatially located between first and second attachment sites, in which the first and second attachment sites are attached to metal nanoparticles. Accordingly, an entity attached to the third attachment site may be disposed between the first metal nanoparticle and the second metal nanoparticle. An entity may be covalently or non-covalently attached to the third attachment site.

An entity disposed between a first metal nanoparticle and a second metal nanoparticle of a plasmonic particle system may comprise an analyte. In some cases, an analyte may comprise a small molecule (i.e., a molecule having a molecular weight of less than 1 kiloDalton) or a macromolecule (i.e., a molecule having a molecular weight of greater than or equal to 1 kiloDalton). In some cases, a small molecule can comprise a pharmaceutical agent, a metabolite, a peptide, an oligonucleotide, a saccharide, a lipid, or a toxin. In some cases, a macromolecule can comprise a synthetic polymer or a biopolymer (e.g., a polypeptide, a polynucleotide, a polysaccharide, a glycoprotein).

An entity disposed between a first metal nanoparticle and a second metal nanoparticle of a plasmonic particle system may comprise a detectable label (e.g., a fluorescent molecule, a luminescent molecule). A fluorescent molecule can include a fluorescent dye or a fluorescent protein. In some cases, one and only one detectable may be disposed between a first metal nanoparticle and a second metal nanoparticle. In some cases, one or more detectable labels (e.g., at least about 1, 2, 3, 4, 5, 10, 15, 20, 25, 30, 40, 50, or more than 50 detectable labels) may be disposed between a first metal nanoparticle and a second metal nanoparticle. Alternatively, no more than about 50, 40, 30, 25, 20, 15, 10, 5, 4, 3, 2, or less than 2 detectable labels may be disposed between a first metal nanoparticle and a second metal nanoparticle. The quantity of detectable labels disposed in a plasmonic particle system may depend upon a desired intensity of signal from the plasmonic particle system (i.e., more detectable labels produce more detectable signal). Without wishing to be bound by theory, presence of a plasmonic particle system may reduce the number of detectable labels necessary to achieve a desired amount of signal. For example, if a fluorescent system without a plasmonic particle system utilizes 10 fluorescent dyes to produce N amount of signal, the same system with a plasmonic particle system may utilize a single fluorescent dye to produce the same N amount of signal.

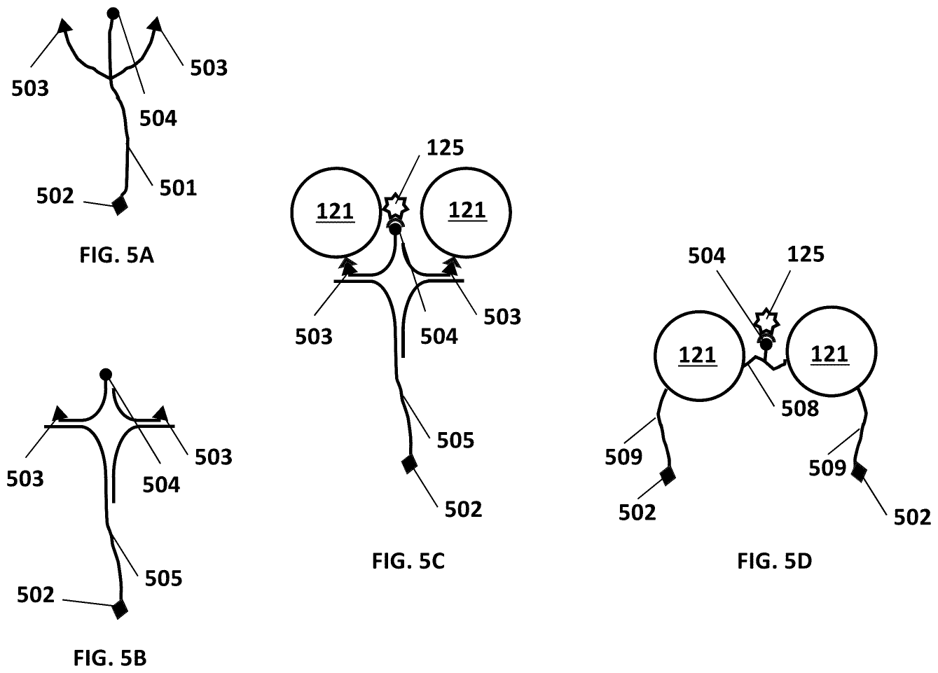

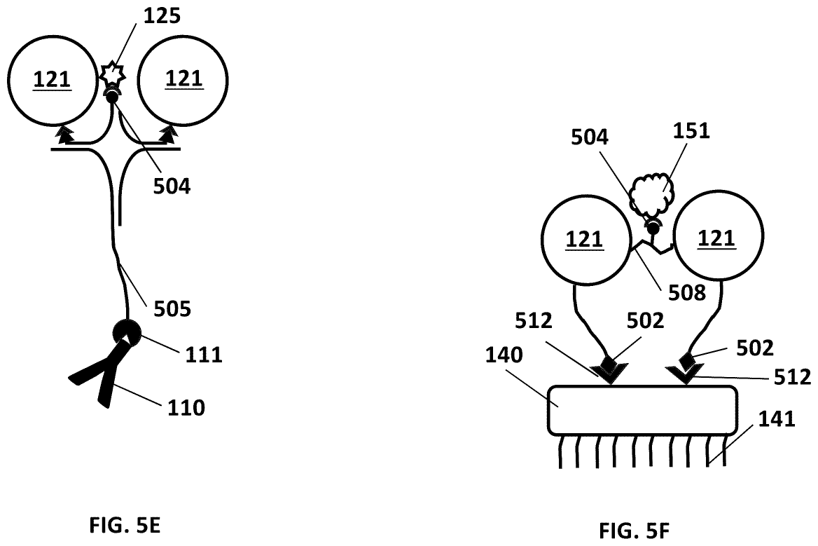

In some cases, two or more metal nanoparticles may be coupled together by linking moieties to form a nanoparticle cluster. In some cases, a nanoparticle cluster may be coupled to an entity (e.g., a detectable label, an analyte) to form a plasmonic particle system. In some cases, it may be advantageous to form a structure comprising a nanoparticle cluster and an entity, then attach the structure to another object (e.g., a particle, an affinity agent, a solid support). FIGS. 5A-5D depict configurations of linking moieties that may be useful for forming plasmonic particle systems and attaching them to objects. FIGS. 5A-5B depict configurations of linking moieties without any attached groups. FIG. 5A depicts a linking moiety 501 comprising a branched polymeric chain. The branched polymeric chain may comprise a plurality of coupling moieties that facilitate attachment of groups to the linking moiety 501, including coupling moieties for attaching metal nanoparticles 503 and one or more coupling moieties for attaching entities 504 (e.g., detectable labels, analytes). The linking moiety may further comprise a coupling moiety 502 for attaching the linking moiety to another object (e.g., a particle, an affinity agent, a solid support). The coupling moieties 502, 503, and 504 may have orthogonal binding specificities to control which objects may be attached to the linking moiety. Orthogonal attachment schemes can include utilizing covalent and non-covalent coupling moieties, or utilizing two differing covalent or non-covalent coupling moieties (e.g., orthogonal Click-type reactants, orthogonal receptor-ligand binding pairs, etc.). FIG. 5B depicts a linking moiety 505 that comprises rigid linkers (e.g., double-stranded nucleic acids, alkenyl or alkynyl moieties). Incorporation of rigid linkers may decrease the spatial degrees of freedom of coupled objects (e.g., metal nanoparticles, detectable labels, analytes) with respect to each other. The rigid linking moiety 505 depicted in FIG. 5B may comprise coupling moieties 502, 503, and 504 as described in FIG. 5A. Coupling moieties may be incorporated into oligonucleotides that assemble into the rigid linker 505. FIG. 5C depicts the rigid linker 505 of FIG. 5B with metal nanoparticles 121 attached to coupling moieties 503 and a detectable label 125 attached to coupling moiety 504, thereby disposing the detectable label 125 between the metal nanoparticles 121. FIG. 5D depicts an alternative configuration in which metal nanoparticles 121 are coupled into a nanoparticle cluster by one or more linking moieties 508. Optionally, a linking moiety 508 may comprise a coupling moiety 504 that facilitates attachment of an entity (e.g., a detectable label 125, an analyte) between the metal nanoparticles 121. One or more additional pendant linking moieties 509 comprising coupling moieties 502 may be attached to the metal nanoparticles, thereby facilitating attachment of the nanoparticle cluster to an object (e.g., a particle, an affinity agent, a solid support).

FIGS. 5E-5F depict the attachment of plasmonic particle systems comprising nanoparticle clusters to objects. FIG. 5E depicts a plasmonic particle system comprising a nanoparticle cluster, as described in FIGS. 5B-5C, attached to an affinity agent 110. Optionally, the coupling moiety 502 described in FIGS. 5B-5C may comprise an antibody-binding protein 111. FIG. 5F depicts a plasmonic particle system comprising a nanoparticle cluster, as described in FIG. 5D, attached to a particle 140 (e.g., a nucleic acid nanoparticle) similar to the particles described in FIG. 1C-1D. The particle 140 comprises a face containing attachment sites, in which each attachment site comprises complementary coupling moiety 512. The nanoparticle cluster is attached to the particle 140 by coupling of coupling moieties 502 to complementary coupling moieties 512.

A composition may be provided with a pendant single-stranded nucleic acid. A pendant single-stranded nucleic acid may be attached to an object such as an array site, a particle, or an affinity agent. A single-stranded nucleic acid may be pendant if it has a sequence of consecutive nucleotides (e.g. at least about 3, 4, 5, 6, 7, 8, 9, 10, 15, 20, 25, 30, 40, 50, or more than 50 consecutive nucleotides) that are not hybridized to a complementary oligonucleotide. Optionally, a pendant single-stranded nucleic acid may comprise an unbound terminal moiety. Optionally, a pendant single-stranded nucleic acid may comprise portions of double-stranded nucleic acid (e.g., a rigid linker moiety).

A composition may be provided a pendant single-stranded nucleic acid to facilitate formation of binding interactions in a system. In some cases, an analyte and/or an anchoring moiety (e.g., a particle, a nucleic acid nanoparticle) may comprise one or more pendant single-stranded nucleic acid strands that facilitate coupling of the analyte and/or anchoring moiety with one or more pedant single-stranded nucleic acids of an array site. In some cases, an array site or a particle attached thereto may comprise a pendant single-stranded nucleic acid docker strand, as set forth herein. In some cases, an affinity agent may comprise or be attached to a pendant single-stranded nucleic acid tether strand, as set forth herein. In some cases, an affinity agent may comprise or be attached to a pendant single-stranded nucleic acid that facilitate attachment of a plasmonic particle system to the affinity agent.

A composition may comprise a particle (e.g., a nucleic acid nanoparticle) comprising two or more faces. In some cases, a pendant single-stranded nucleic acid may be attached to a face of the two or more faces of the particle. In some cases, a particle may comprise a first face and a second face, in which a first pendant single-stranded nucleic acid is attached to the first face and a second single-stranded nucleic acid is attached to the second face. For example, FIG. 1D may depict a nanoparticle 140 having a first face comprising a plurality of surface-coupling oligonucleotides 141 and a second face comprising a pendant docker strand 146. In this configuration, the face containing the docker strand 146 is not substantially distal to the face containing the surface-coupling oligonucleotides 141. Alternatively, the docker strand 146 could be coupled to the face containing the metal nanoparticles 121 and analyte 151, thereby orienting the docker strand 146 on a face that is substantially distal from the face containing the surface-coupling oligonucleotides 141.

An object (e.g., a particle, an affinity agent, an array site) may be provided with at least about 1, 2, 3, 4, 5, 6, 7, 8, 9, 10, 15, 20, 30, 40, 50, 100, 500, 1000, 10000, 100000, 1000000, or more than 1000000 pendant single-stranded nucleic acids. Alternatively or additionally, an object may be provided with no more than about 1000000, 100000, 10000, 1000, 500, 100, 50, 40, 30, 20, 15, 10, 9, 8, 7, 6, 5, 4, 3, 2 or less than 2 pendant single-stranded nucleic acids. The provided quantity of pendant single-stranded nucleic acids for an object may depend upon the intended use of the nucleic acids. For example, pendant single-stranded nucleic acids for attaching analytes or anchoring moieties to array sites may be provided in larger quantities (e.g., at least 10 or more pendant single-stranded nucleic acids). In another example, docker or tether strands may be provided in smaller quantities (e.g., between about 1 and 10 nucleic acids).

A pendant single-stranded nucleic acid may be configured to hybridize to a complementary pendant single-stranded nucleic acid (e.g., a docker strand hybridizing to a tether strand, a surface-coupling oligonucleotide hybridizing to a surface-coupled oligonucleotide). Accordingly, the complementary pendant single-stranded nucleic acids may comprise nucleotide sequences that are configured to form a binding interaction with a complementary nucleotide sequence, and nucleotide sequences that are not configured to form a binding interaction. A nucleotide sequence that is configured to form a binding interaction may have at least about 4, 5, 6, 7, 8, 9, 10, 11, 12, 13, 14, 15, 16, 17, 18, 19, 20, 25, 30, 35, 40, 45, 50, 60, 70, 80, 90, 100, or more than 100 nucleotides. Alternatively or additionally, a nucleotide sequence that is configured to form a binding interaction may have no more than about 100, 90, 80, 70, 60, 50, 45, 40, 35, 30, 25, 20, 19, 18, 17, 16, 15, 14, 13, 12, 11, 10, 9, 8, 7, 6, 5, 4, or less than 4 nucleotides. The quantity of nucleotides in a nucleotide sequence that is configured to form a binding interaction may be determined by the intended strength of the binding interaction. For example, surface-coupling binding interactions may be intended to have a stronger interaction, so a larger quantity of nucleotides (e.g., at least 10, 15, 20, or more than 20 nucleotides) of complementarity may be preferable. In another example, an interaction between a docker and tether strand may be intended to have a weaker interaction, so a smaller quantity of nucleotides (e.g., no more than 10 nucleotides) of complementarity may be preferable.

An object (e.g., a particle, an affinity agent, an array site) may comprise an attachment site for a plasmonic particle system or a component thereof (e.g., a metal nanoparticle, a detectable label, an analyte). An attachment site for a plasmonic particle system or a component thereof may comprise a linking moiety and/or a spacing moiety. A spacing moiety may be provided to spatially separate a component of the plasmonic particle system from a surface (e.g., a surface of a particle, a surface of a solid support). A spacing moiety may comprise a flexible linker (e.g., a synthetic polymer chain, a single-stranded nucleic acid, a peptide chain) or a rigid linker (e.g., a double-stranded nucleic acid, an alkenyl moiety, an alkynyl moiety). A spacing moiety may provide a separation distance between a component of a plasmonic particle system and a surface of at least about 5 nanometers (nm), 10 nm, 15 nm, 20 nm, 25 nm, 30 nm, 40 nm, 50 nm, or more than 50 nm. Alternatively or additionally, a pacing moiety may provide a separation distance between a component of a plasmonic particle system and a surface of no more than about 50 nm, 40 nm, 30 nm, 25 nm 20 nm, 15 nm 10 nm, 5 nm, or less than 5 nm.

Methods provided herein may comprise one or more steps of: i) contacting a composition set forth herein with a photon of light, and/or ii) detecting a photon of light radiated (e.g., reflected, refracted, emitted) from a composition set forth herein. Accordingly, a composition set forth herein may further comprise a photon of light or a plurality thereof. A composition may comprise a photon of light that is incident upon a metal nanoparticle or an entity (e.g., a detectable label, an analyte) of a plasmonic particle system. A composition may comprise a photon of light that is radiated from a metal nanoparticle or an entity (e.g., a detectable label, an analyte) of a plasmonic particle system. A composition may comprise a plurality of photons, in which individual photons of the plurality of photons comprise substantially the same wavelength. For example, a light field may contact a plasmonic particle system comprising a detectable label, in which the photons of the light field have a wavelength at the excitation wavelength of the detectable label. A composition may comprise a plurality of photons, in which individual photons of the plurality of photons comprise differing wavelengths. For example, spectroscopic or fluorescent method may involve the simultaneous illumination of light upon and radiation of light from a plasmonic particle system, in which the illuminating light and radiated light differ with respect to wavelength. Some spectroscopic or fluorescent methods may utilize multiple wavelengths of light, in which the wavelength of light illuminating or radiating from a plasmonic particle system may vary with respect to time.

It may be useful to attach (e.g., covalently, non-covalently) a field-orientable particle to a macromolecule or analyte of a plasmonic particle system. The field-orientable particle may comprise a magnetic nanoparticle or an electrically-charged particle. Altering a field (e.g., a magnetic field, an electric field) surrounding a field-orientable particle may alter the orientation of the field-orientable particle and a macromolecule or analyte attached thereto.

In some compositions, a macromolecule or analyte may be disposed between two or more metal nanoparticles of a plasmonic particle system. A macromolecule or analyte may comprise a polymeric chain, including linear, branched, or dendrimeric chains. A polymeric chain may comprise a plurality of residues that are covalently bonded to form the polymeric chain. A polymeric chain may comprise a repeating sequence of residues (e.g., a block copolymer, certain oligosaccharides) or may comprise a random sequence of residues (e.g., certain polypeptides, polynucleotides, or polysaccharides). A polymeric chain may comprise secondary and/or tertiary structures that provide a three-dimensional conformation or morphology to the polymeric chain. In some cases, a polymeric chain may be provided to a plasmonic particle system with a secondary and/or tertiary structure. In some cases, a polymeric chain may be provided to a plasmonic particle system with a partially denatured secondary or tertiary structure. In some cases, a polymeric chain may be provided to a plasmonic particle system with a fully denatured secondary or tertiary structure. In some cases, a partially denatured polymeric chain may be provided to a plasmonic particle system. In some cases, a fully denatured polymeric chain may be provided to a plasmonic particle system. Accordingly, a composition provided herein may further comprise a denaturing agent or a chaotrope.

A method may comprise a step of contacting a macromolecule or analyte with a denaturing agent or chaotrope, or heating the macromolecule or analyte, before attaching the macromolecule or analyte to a plasmonic particle system. A method may comprise a step of contacting a macromolecule or analyte with a denaturing agent or chaotrope, or heating the macromolecule or analyte, after attaching the macromolecule or analyte to a plasmonic particle system. Any method step set forth herein may occur in the presence of a denaturing agent or chaotrope, or in the presence of heat. In some cases, a method step may occur in an absence of a denaturing agent, chaotrope, or heat. If a macromolecule or analyte is provided in a partially- or fully-denatured state, it may be advantageous to periodically provide a denaturing agent to maintain the macromolecule or analyte in the partially- or fully-denatured state.

In another aspect, provided herein is a method, comprising: a) coupling a binding reagent to an analyte, wherein the analyte is immobilized on a solid support, and wherein the binding reagent comprises a nanoparticle cluster, wherein the nanoparticle cluster comprises a first metal nanoparticle, a second metal nanoparticle, and a light-emitting molecule (e.g., a fluorescent dye, a fluorescent protein, a luminescent molecule) disposed between the first metal nanoparticle and the second metal nanoparticle, b) contacting the nanoparticle cluster with light, and c) detecting an optical signal (e.g., a fluorescent signal, a luminescent signal) from the light-emitting molecule, thereby identifying an address of the solid support containing the binding reagent coupled to the analyte.

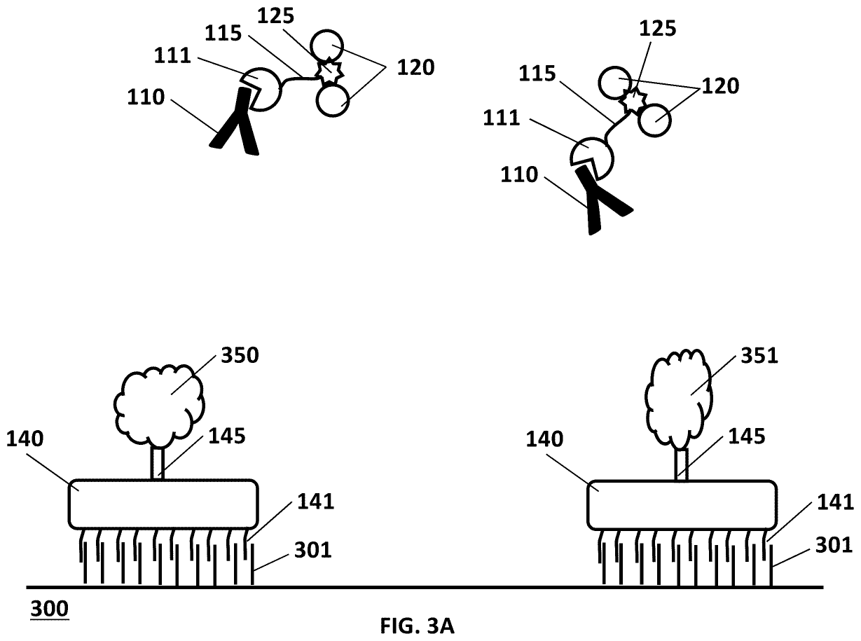

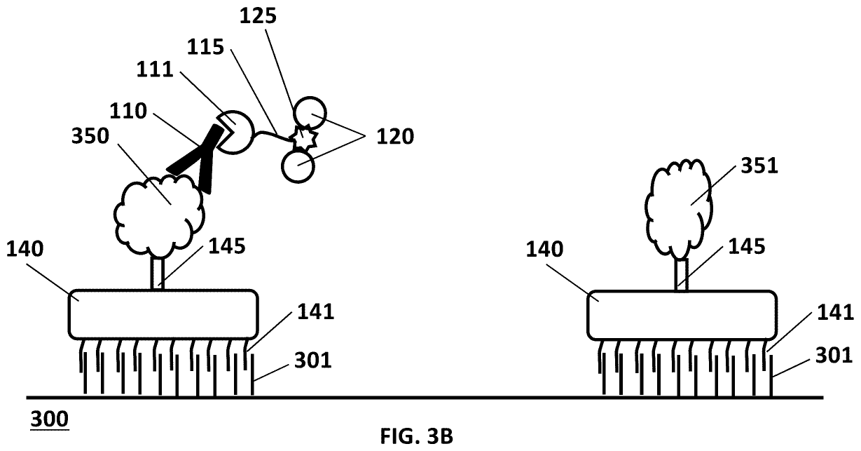

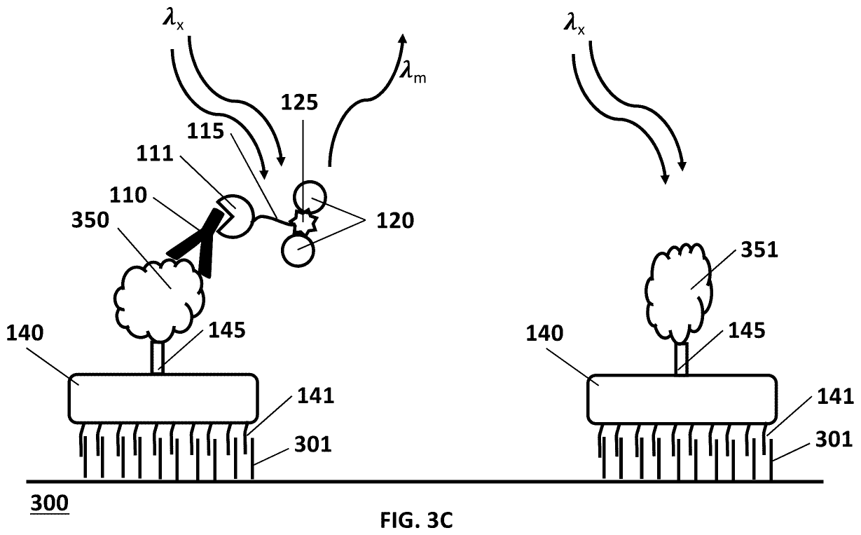

FIGS. 3A-3C illustrate aspects of utilizing a binding reagent comprising a plasmonic particle system. FIG. 3A depicts contacting of a plurality of binding reagents, optionally by delivering a fluidic medium comprising the plurality of binding reagents, to a solid support 300 comprising an array of analytes, in which each individual binding reagent of the plurality of binding reagents comprises a plasmonic particle system. Aspects of binding reagents containing plasmonic particle systems are described herein, for example in FIGS. 1A and 1B. The array of analytes comprises a plurality of sites, optionally configured such that each site is an optically resolvable distance from any other site of the plurality of sites. Each individual site comprises a plurality of surface-coupled coupling moieties 301. One or more surface-coupled coupling moieties 301 are coupled to surface-coupling moieties 141 of a particle 140 (e.g., a nucleic acid nanoparticle), thereby attaching the particle to the individual site. At a first site, a first analyte 350 is attached to a particle 140, optionally by a spacing moiety 145. At a second site, a second analyte 351 is attached to a particle 140, optionally by a spacing moiety 145. In some cases, the first analyte 350 may differ from the second analyte 351 (e.g., with respect to type of analyte, with respect to species of analyte, with respect to a size of the analyte, with respect to a morphology of the analyte, etc.). FIG. 3B depicts a configuration in which a binding reagent has bound to the first analyte 350 by coupling of an affinity agent 110 to the first analyte 350. A binding reagent has not bound to the second analyte 351. Optionally, the solid support 300 has been rinsed to remove any unbound binding reagents. FIG. 3C depicts contacting the binding reagent with a light field comprising photons of light having a wavelength at the excitation wavelength, λₓ, of the detectable label 125. The detectable label 125 radiates a photon of wavelength λₘ that may be detected by a sensing device, thereby facilitating detection of an optical signal at an array address corresponding to the site at which the first analyte 350 is bound.

Without wishing to be bound by theory, the presence of a plasmonic particle system containing a light-emitting molecule may enhance the amount of an optical signal emitted by the light-emitting molecule. Accordingly, the presence of a plasmonic particle system containing the light-emitting molecule may facilitate one or more of: i) increasing a magnitude of an optical signal from an array address containing the light-emitting molecule, ii) increasing a magnitude of an optical signal from an address containing the light-emitting molecule with respect to a background or average optical signal, iii) decreasing a necessary energy or power input of excitation light to detect a fixed amount of optical signal from an array address containing the light-emitting molecule, iv) decreasing a necessary time of providing excitation light to detect a fixed amount of optical signal from an array address containing the light-emitting molecule, and v) decreasing a necessary amount of signal collection time to detect an optical signal from an array address containing the light-emitting molecule.

In another aspect, provided herein is an array, comprising: a) a solid support comprising a plurality of optically resolvable sites, b) at each individual site of the plurality of optically resolvable sites, one and only one analyte coupled to each individual site, and c) binding reagents coupled to analytes at sites of the plurality of optically resolvable sites, in which each individual binding reagent comprises no more than 5 detectable labels (e.g., no more than 4, 3, 2, or 1 detectable labels). In some cases, each individual binding reagent can comprise one and only one detectable label.

In some cases, each site of a plurality of optically-resolvable sites may be separated from any other site of the plurality of optically-resolvable sites by a distance of at least about 100 nanometers (nm), 200 nm, 300 nm, 400 nm, 500 nm, 600 nm, 700 nm, 800 nm, 900 nm, 1 micron (μm), 1.5 μm, 2 μm, 3 μm, 4 μm, 5 μm, 10 μm, or more than 10 μm. Alternatively or additionally, each site of a plurality of optically-resolvable sites may be separated from any other site of the plurality of optically-resolvable sites by a distance of no more than about 10 μm, 5 μm, 4 μm, 3 μm, 2 μm, 1.5 μm, 1 μm, 900 nm, 800 nm, 700 nm, 600 nm, 500 nm, 400 nm, 300 nm, 200 nm, 100 nm, or less than 100 nm. Accordingly, each analyte coupled to the array may be separated from any other analyte coupled to the array by at least about 100 nm, 200 nm, 300 nm, 400 nm, 500 nm, 600 nm, 700 nm, 800 nm, 900 nm, 1 micron (μm), 1.5 μm, 2 μm, 3 μm, 4 μm, 5 μm, 10 μm, or more than 10 μm. Alternatively or additionally, each analyte coupled to the array may be separated from any other analyte coupled to the array by no more than about 10 μm, 5 μm, 4 μm, 3 μm, 2 μm, 1.5 μm, 1 μm, 900 nm, 800 nm, 700 nm, 600 nm, 500 nm, 400 nm, 300 nm, 200 nm, 100 nm, or less than 100 nm.

In another aspect, provided herein is a method comprising: a) providing an array as set forth herein, b) illuminating the plurality of optically resolvable sites with a light field, and c) detecting at each individual site of the sites of the plurality of optically resolvable sites a detectable signal from a binding reagent of the binding reagents.

An address of an array (e.g., an address containing a site, an address containing an analyte, an address containing a plasmonic particle system) may be illuminated with light for at least about 0.1 microseconds (μs), 1 μs, 10 μs, 100 μs, 1 millisecond (ms), 10 ms, 100 ms, 1 second(s), 10 s, or more than 10 s. Alternatively or additionally, an address of an array may be illuminated with light for no more than about 10 s, 1 s, 100 ms, 10 ms, 1 ms, 100 μs, 10 μs, 1 μs, or less than 1 μs. An optical signal may be detected at an address of an array (e.g., an address containing a site, an address containing an analyte, an address containing a plasmonic particle system) for at least about 0.1 microseconds (μs), 1 μs, 10 μs, 100 μs, 1 millisecond (ms), 10 ms, 100 ms, 1 second(s), 10 s, or more than 10 s. Alternatively or additionally, an optical signal may be detected at an address of an array for no more than about 10 s, 1 s, 100 ms, 10 ms, 1 ms, 100 μs, 10 μs, 1 μs, or less than 1 μs.

An address of an array (e.g., an address containing a site, an address containing an analyte, an address containing a plasmonic particle system) may be illuminated with a light field having a power density of at least about 10⁻² Watts per square centimeter (W/cm²), 10⁻¹ W/cm², 1 W/cm², 10 W/cm², 10² W/cm², 10³ W/cm², 10⁴ W/cm², 10⁵ W/cm², 10⁶ W/cm², 10⁷ W/cm², 10⁸ W/cm², 10⁹ W/cm², or more than 10⁹ W/cm². Alternatively or additionally, an address of an array may be illuminated with a light field having a power density of no more than about 10⁹ W/cm², 10⁸ W/cm², 10⁷ W/cm², 10⁶ W/cm², 10⁵ W/cm², 10⁴ W/cm², 10³ W/cm², 10² W/cm², 10 W/cm², 1 W/cm², 10⁻¹ W/cm², 10⁻² W/cm², or less than 10⁻² W/cm².

A method of detecting presence or absence of bound affinity agents coupled to plasmonic particle system at array sites may comprise contacting or illuminating an array containing the array sites with a light field. In some cases, an entire array may be simultaneously illuminated. In other cases, a subset of array sites of an array of sites may be illuminated by a light field. In some cases, contacting an array site with light may comprise rastering a light field across the site of the plurality of sites. In some cases, contacting a plurality of sites with light may comprise rastering a light field across each site of the plurality of sites. In some cases, rastering a light field across each site of a plurality of sites may comprise sequentially illuminating each site of the plurality of sites. In other cases, rastering a light field across each site of a plurality of sites may comprise simultaneously illuminating a subset of the plurality of sites with a light field.

In another aspect, provided herein is a method, comprising: a) providing an analyte or macromolecule immobilized on a solid support, wherein the analyte or macromolecule is disposed on the solid support between a first metal nanoparticle and a second metal nanoparticle, b) contacting the analyte or macromolecule immobilized on the solid support with light, c) detecting scattering of the light contacted to the analyte or macromolecule immobilized on the solid support, and d) based upon the scattering of the light contacted to the analyte or macromolecule, identifying a structure of the analyte or macromolecule immobilized on the solid support.

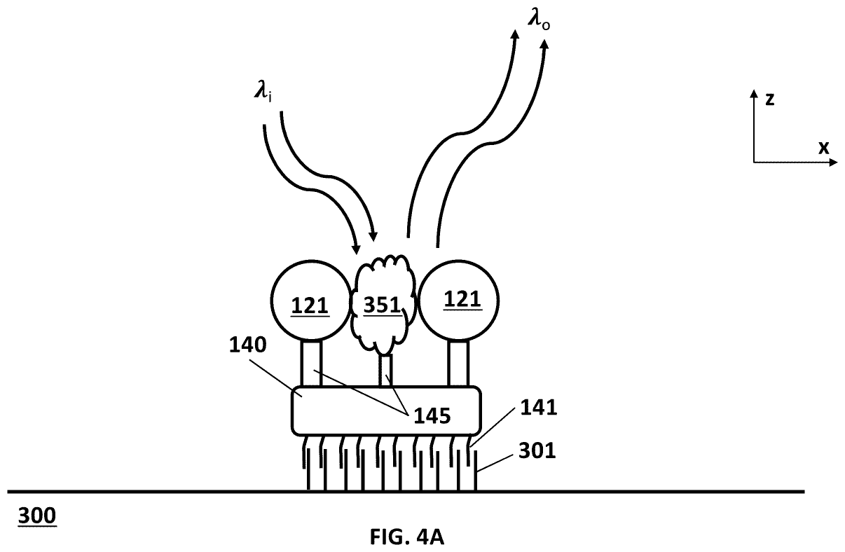

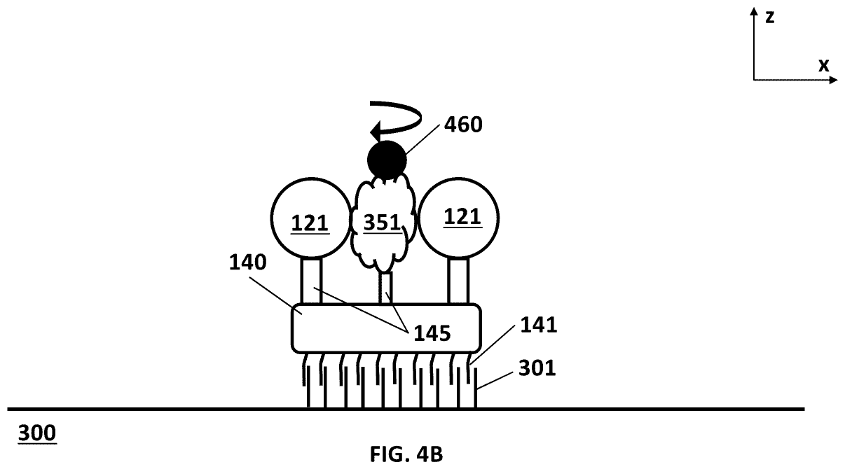

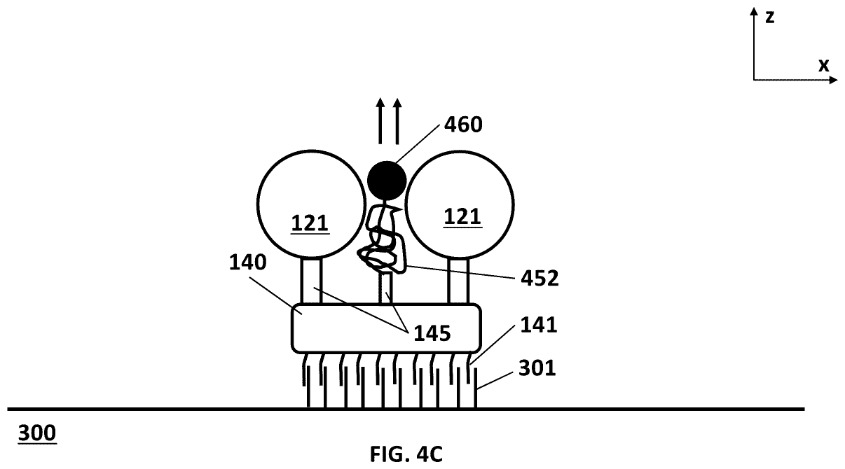

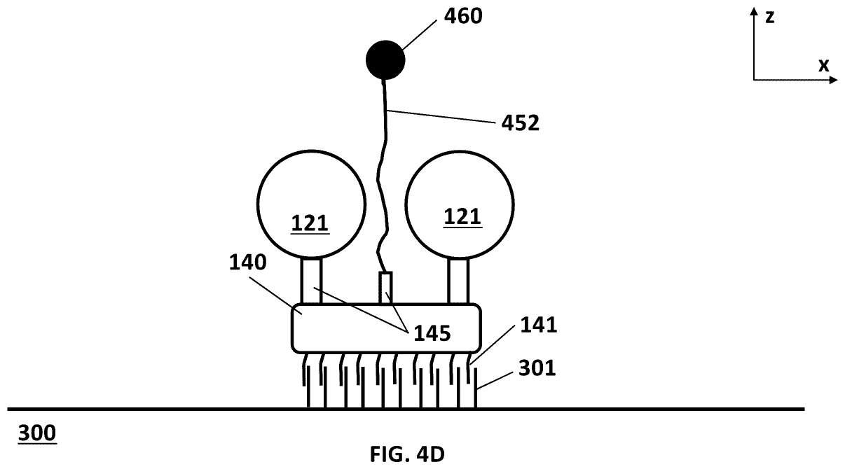

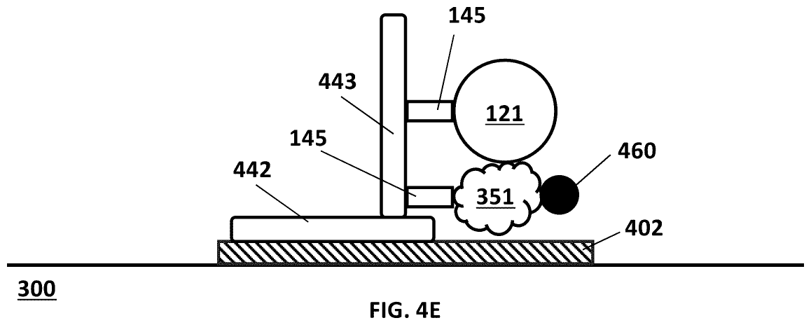

FIGS. 4A-4D depicts steps of a method of characterizing an analyte utilizing a plasmonic particle system. FIG. 4A depicts a solid support 300 containing a site with a coupled analyte 351. The analyte 351 is bound by a similar configuration as described in FIG. 3A. The particle 140 coupled to the analyte 351 further comprises a plasmonic particle system comprising two metal nanoparticles 121. As shown in FIG. 4A, the analyte 351 may be illuminated with light of wavelength λᵢ and may radiate light of wavelength λₒ. Without wishing to be bound by theory, a shift in wavelength between λᵢ and λₒ may occur due to a surface-enhanced Raman effect of the analyte 351 in the presence of the plasmonic particle system. FIG. 4B depicts a similar configuration to FIG. 4A, with a field-orientable particle 460 (e.g., a magnetic particle, an electrically-charged particle) attached to the analyte 351. Rotation of the field-orientable particle 460 in the presence of a force-generating field (e.g., a magnetic field, an electric field) may alter an orientation of the analyte 351 due to rotation of some or all of the structure of the analyte 351. During or after the alteration of the orientation of the analyte 351 by rotation of the field-orientable particle 460, the illumination and detection described for FIG. 4A may be repeated. FIG. 4C depicts a similar configuration to that of FIG. 4B, in which the field-orientable particle 460 is translated in the z-axis direction in the presence of a force-generating field (e.g., a magnetic field, an electric field). The translation of the field-orientable particle 460 may alter an orientation of the analyte 452 due to translation of some or all of the structure of the analyte 452. As shown in FIG. 4D, if the analyte comprises a compacted or folded structure (e.g., a secondary or tertiary structure), translation of a portion of the analyte 452 may facilitate unfolding or denaturation of the structure of the analyte 452.

FIG. 4E depicts an alternative configuration of the plasmonic particle system depicted in FIGS. 4A-4D. A metal pad 402, optionally comprising a noble metal, as set forth herein, is disposed on a solid support 300. A particle (e.g., a nucleic acid nanoparticle) may be attached to the metal pad 402 such that attachment sites (optionally comprising spacing moieties 145) are oriented in a substantially parallel direction to the surface of the metal pad 402 or solid support 300. Optionally, a complex may be formed comprising two or more coupled particles (e.g., 442 and 443). Alternatively, it may be possible to provide a single particle that attaches to the metal pad 402 or solid support 300 and properly orients the analyte 351 such that the analyte 351 is disposed between the metal pad 402 and the metal nanoparticle 121. Optionally, a field-orientable particle may be attached to the analyte 351. Preferably, the metal pad 402 may have a characteristic dimension (e.g., length, width, thickness) that is comparable to the characteristic dimensions of metal nanoparticles 121 set forth herein.

A method may comprise a step of altering the orientation of an analyte or macromolecule that is disposed between metal nanoparticles of a plasmonic particle system. Altering an orientation can include rotating the analyte or macromolecule, translating the analyte or macromolecule, or a combination thereof. Without wishing to be bound by theory, the orientation of an analyte or macromolecule may be altered to provide increased spectroscopic or optical information about the analyte or macromolecule. Altering an orientation of a macromolecule or analyte may disrupt existing secondary or tertiary structures, or form secondary or tertiary structures of the macromolecule or analyte. Changes in the two- or three-dimensional morphology of the macromolecule or analyte may produce spectroscopic or optical changes that may provide increased information about the structure, composition, and/or identity of the macromolecule or analyte. In some cases, a method may comprise one or more cycles of: i) altering an orientation of an analyte or macromolecule, ii) contacting the analyte or macromolecule with light, and iii) detecting scattering of the light contacted to the analyte or macromolecule. Steps i)-iii) may be repeated for at least about 2, 3, 4, 5, 10, 20, 30, 40, 50, 100, 500, 1000, or more than 1000 cycles. Information from each cycle may be combined to provide an identity or other characterization of the analyte or macromolecule.

In some cases, it may be preferable to provide an analyte or macromolecule that comprises a secondary or tertiary structure. Alternatively, it may be preferable to provide an analyte or macromolecule that does not comprise a secondary or tertiary structure (e.g., a linearized or unfolded polymeric chain). Accordingly, a method may comprise a step of disrupting a secondary or tertiary structure (e.g., in the presence of a denaturing agent, chaotrope, or heat). Disrupting a secondary or tertiary structure may comprise partially or fully denaturing the secondary or tertiary structure of an analyte or macromolecule. A step of disrupting a secondary or tertiary structure of a macromolecule or analyte can occur before the macromolecule or analyte is contacted with light. Alternatively, a step of disrupting a secondary or tertiary structure of a macromolecule or analyte can occur after the macromolecule or analyte is contacted with light. If disruption of the secondary or tertiary structure occurs after the macromolecule or analyte is contacted with light, the method of contacting the macromolecule or analyte with light and measuring light radiated from the macromolecule or analyte may be repeated.

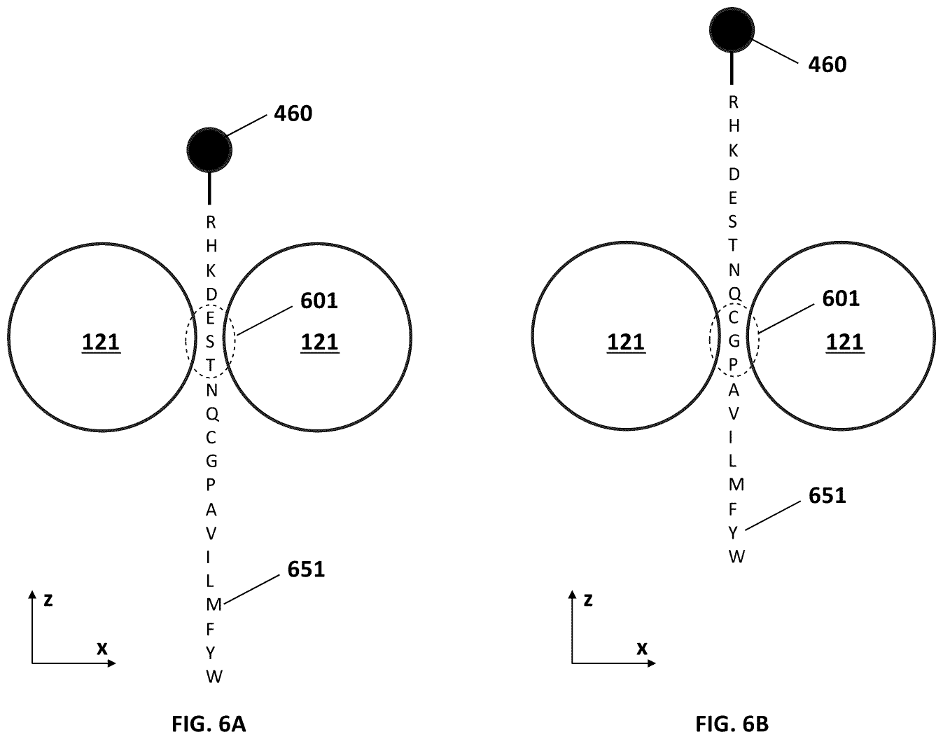

A method may comprise a step of translating a polymeric chain through a nanoparticle cluster of a plasmonic particle system. FIGS. 6A-6B depict a method of translating a linearized or extended polymeric chain through a separation gap between two metal nanoparticles. FIG. 6A depicts a first configuration of a plasmonic particle system at a first time. A linearized or extended polypeptide chain 651 is attached to a field-orientable particle 460. The linearized or extended polypeptide chain 651 is disposed between two metal nanoparticles 121 of a plasmonic particle system. If the plasmonic particle system is contacted with light, only a small region of the polypeptide chain 651 (e.g., the amino acid sequence EST highlighted in oval 601) will be in sufficient proximity to the metal nanoparticle 121 to form a plasmonic interaction. Spectroscopic information (e.g., IR, UV, or Raman spectra) obtained at the first timepoint for the small region may be sufficient to identify the sequence of residues interacting with the impinging light field. FIG. 6B depicts a second configuration of a plasmonic particle system at a second time. The field-orientable particle 460 has been translated upward in the z-axis direction, thereby translating the polypeptide chain 651 through the separation gap between the metal nanoparticles 121. A different region of the polypeptide chain 651 (e.g., the amino acid sequence CGP highlighted in oval 601) is disposed between the two metal nanoparticles 121. Additional spectroscopic information may be obtained at the second timepoint, thereby facilitating identification of additional residue sequences present in the polypeptide chain 651. Repetition of a spectroscopic method as the polypeptide chain 651 is translated through the separation gap between the two metal nanoparticles 121 may provide sufficient information to identify a protein species or proteoform of the polypeptide chain 651, or provide a partial or complete amino acid sequence of the polypeptide chain 651.

Provided herein is a method for identifying a macromolecule or analyte with an optical signature obtained utilizing a plasmonic particle system. In an aspect, a first analyte or macromolecule may be distinguished from a second analyte or macromolecule by differences between an optical signature of the first analyte or macromolecule and an optical signature of the second analyte or macromolecule. Likewise, a first analyte or macromolecule may be assigned a same identity as a second analyte or macromolecule if the first analyte or macromolecule and the second analyte or macromolecule have substantially the same optical signature.

An optical signature of an analyte or macromolecule may comprise measurements of optical signal intensity over a range of frequencies (e.g., IR or UV spectra) or over a range of wavenumbers (e.g., a Raman spectrum). An optical signature of an analyte or macromolecule may comprise a plurality of measurements. For example, a spectral measurement may be associated with each individual orientation of a sequence of analyte or macromolecule orientations produced by altering the orientation of a field-orientable particle. Likewise, a spectral measurement may be associated with each individual structure of a sequence of analyte or macromolecule structures produced by disrupting or forming a secondary or tertiary structure of the analyte or macromolecule.

An Raman spectrum may be obtained in a range of wavenumber from about 4000 cm⁻¹ to about 10 cm⁻¹, or a subrange thereof, such as about 4000 cm⁻¹ to about 100 cm⁻¹, about 4000 cm⁻¹ to about 1000 cm⁻¹, about 3000 cm⁻¹ to about 10 cm⁻¹, about 3000 cm⁻¹ to about 100 cm⁻¹, about 3000 cm⁻¹ to about 1000 cm⁻¹, about 2000 cm⁻¹ to about 10 cm⁻¹, about 2000 cm⁻¹ to about 100 cm⁻¹, about 2000 cm⁻¹ to about 1000 cm⁻¹, about 1000 cm⁻¹ to about 10 cm⁻¹, or about 1000 cm⁻¹ to about 100 cm⁻¹. A spectroscopic measurement may be obtained in the ultraviolet range, visible, infrared range, or a combination thereof. A spectroscopic measurement may be obtained in a range from about 100 nm to about 400 nm, about 100 nm to about 700 nm, about 100 nm to about 1000 nm, about 100 nm to about 2500 nm, about 400 nm to about 700 nm, about 400 nm to about 1000 nm, about 400 nm to about 2500 nm, about 700 nm to about 1000 nm, about 700 nm to about 2500 nm, or about 1000 nm to about 2500 nm.

Optical signatures for differing analyte or macromolecules may be collected in a database of optical signatures. A method may comprise a step of comparing an optical signature of an analyte or macromolecule, as measured by a method set forth herein, and identifying from a database an analyte or macromolecule with a closest matching optical signature (e.g., as determined by a statistical measure of variance or deviation).

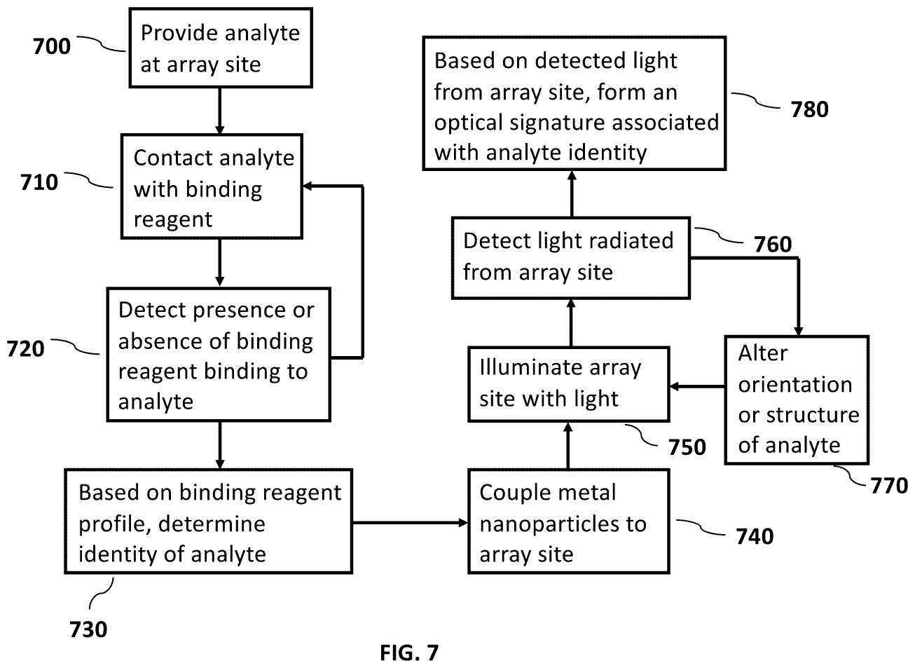

FIG. 7 depicts a schematic of a method of obtaining an optical signature for a particular analyte. It may be especially useful to perform the method of FIG. 7 on a single-analyte array, as set forth herein. The high multiplexity of a single-analyte array can facilitate replicate optical signature detection of a single species of analyte or macromolecule, or can facilitate simultaneous optical signature detection of many differing species of analytes or macromolecules.

Turning to FIG. 7, a first step 700 of a method of collecting an optical signature may comprise providing an analyte or macromolecule at an identifiable address on a solid support, such as an array site. Optionally, the analyte or macromolecule may be attached to a particle that couples the analyte or macromolecule to the solid support or a site thereof. In a second step 710, the analyte or macromolecule may be contacted with a binding reagent or a pool of binding reagents, thereby facilitating binding of the binding reagent to the analyte or macromolecule. In a third step 720, presence or absence of binding of the binding reagent to the analyte or macromolecule may be detected (e.g., via presence or absence of an optical signature from the binding reagent) at the address containing the analyte or macromolecule. Optionally, steps 710 and 720 may be repeated (e.g., with the same binding reagent, with a differing binding reagent) and the results of each cycle (i.e., presence or absence of binding of each binding reagent) may be collected into a binding reagent profile. In a fourth step 730, based upon a binding reagent profile containing data regarding presence or absence of binding of one or more binding reagents, the analyte or macromolecule may be identified according to a method set forth herein.

Continuing with FIG. 7, after an analyte or macromolecule has been identified, a plasmonic particle system may be formed 740 at the address containing the analyte or macromolecule. Forming the plasmonic particle system may comprise attaching two or more metal nanoparticles to two or more attachment sites at the address containing the analyte or macromolecule. Preferably, the two or more metal nanoparticles can be attached to a particle (e.g., a nucleic acid nanoparticle) that positions the metal nanoparticles such that the analyte or macromolecule is disposed between the two or more metal nanoparticles. In a sixth step 750, after forming the plasmonic particle system 740, the analyte or macromolecule may be illuminated by light from a light source (e.g., a laser, a diode, a lamp, a bulb, a luminescent source, etc.). In a seventh step 760, light radiated (e.g., reflected, refracted, emitted) from the plasmonic particle system may be detected on a suitable detector (e.g., an infrared (IR) detector, an ultraviolet (UV) detector, a Raman detector, etc.). Detection by the detector may occur over a range of wavelengths or wavenumbers, thereby providing a spectrum for the analyte or macromolecule under the tested conditions. Optionally, the illumination step 750 and the detection step 760 may be repeated one or more times. Optionally, the analyte or macromolecule may be altered 770 before repeating an illumination step 750 or detection step 760. Altering the analyte or macromolecule can include altering an orientation of the macromolecule (e.g., by re-orienting an attached field-orientable particle), disrupting a secondary or tertiary structure of the analyte or macromolecule (e.g. in the presence of a denaturing agent, chaotrope, or heat), or forming a secondary or tertiary structure of the analyte or macromolecule (e.g., in the absence of a denaturing agent, chaotrope, or heat). In a final step 780, one or more detected data (e.g., one or more spectra) may be collected to form an optical signature for the analyte or macromolecule.

The skilled person can readily recognize that the method described in FIG. 7 can be varied. For example, steps 740-780 can be performed initially, then the metal nanoparticles may be detached from an array site before performing steps 700-730. Such a configuration would favor attachment of metal nanoparticles by a separable attachment system, such as photocleavable linking moieties, oligonucleotides, or enzymatically-cleavable linking moieties (e.g., cleaving of a nucleic acid linker by a restriction enzyme, cleaving of a peptide linker by a protease, etc.).

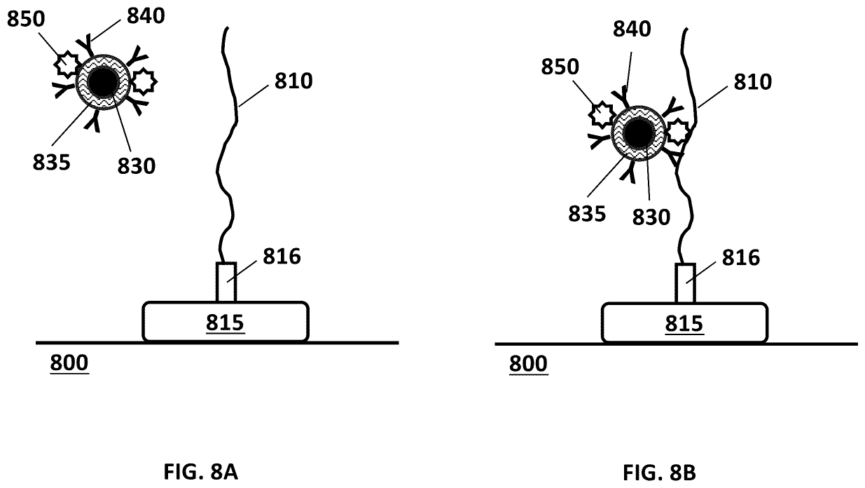

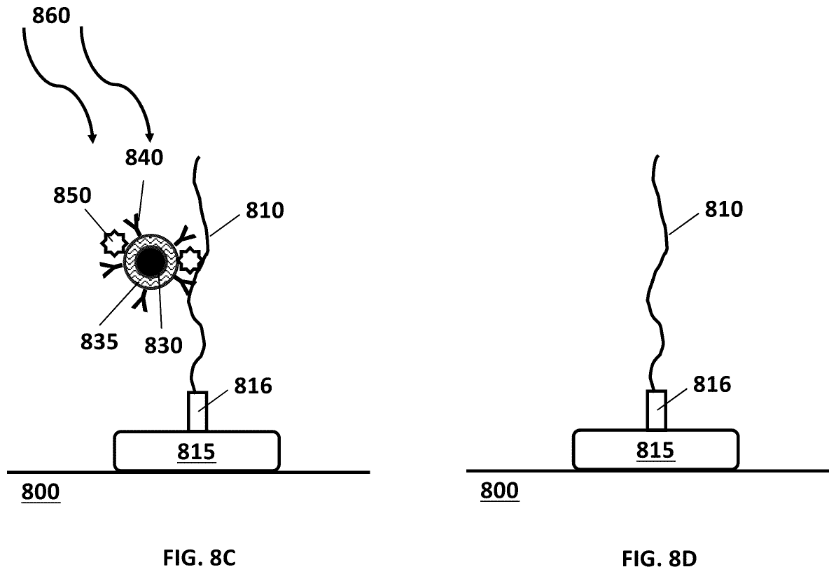

Further provided herein is a method of dissociating a binding reagent comprising a metal nanoparticle from an analyte. FIGS. 8A-8D depicts steps of a method of dissociating a binding reagent from an analyte utilizing a plasmonic interaction. FIG. 8A depicts a first step comprising contacting a binding reagent to an analyte 810. The analyte is immobilized on a solid support 800, optionally by attachment to a particle 815. The binding reagent comprises a metal nanoparticle 830 (e.g., a noble metal nanoparticle such as rhenium, ruthenium, rhodium, palladium, silver, osmium, iridium, platinum, gold, copper, or combinations or alloys thereof) surrounded by an optional passivating layer 835 (e.g., a PEGylated layer, alkyl moieties, fluoroalkyl moieties, etc.). The metal nanoparticle 830 is attached to one or more affinity agents 840 and one or more detectable labels 850 (e.g., fluorescent dyes, luminescent moieties). Alternatively, configurations of binding reagents such as those shown in FIGS. 1A-1B may be useful for a method of binding reagent dissociation. The binding reagent is contacted to the analyte 810, optionally in a fluidic medium, thereby facilitating coupling of the affinity agent 840 of the binding reagent to the analyte 810. FIG. 8B depicts a second step of a method, in which an affinity agent 840 of the binding reagent is coupled to the analyte 810. It will be recognized that a method of dissociating a binding reagent from an analyte can readily be adapted to methods dissociating pluralities of binding reagents from arrays containing pluralities of analytes.

Optionally, a method of dissociating a binding reagent may comprise a step of detecting a detectable signal from the detectable label 850 at an address of the solid support where the analyte 810 is immobilized. In some cases, a method of detecting a detectable signal from a detectable label 850 may comprise illuminating a plasmonic particle system, as set forth herein.

FIG. 8C depicts a third step of a method comprising illuminating the metal nanoparticle 830 with a light field 860. Without wishing to be bound by theory, the light field 860 may comprise photons of a wavelength that is absorbed by the metal nanoparticle 830, thereby resulting in plasmonic dissipation of heat. It may be useful to illuminate the metal nanoparticle with light having a wavelength in the infrared portion of the electromagnetic spectrum. FIG. 8D depicts a fourth step of a method, in which the binding reagent has dissociated from the analyte 810 and optionally diffused away from the address where the analyte 810 is immobilized or has been convectively removed by transfer of a fluidic medium containing the binding reagent away from the solid support 800.

Useful methods of forming binding reagents containing nanoparticles, including metal nanoparticles, can be found in U.S. Pat. No. 11,692,217 and Stawicki, C. M., et al. “Modular Fluorescent Nanoparticle DNA Probes for Detection of Peptides and Proteins.” Sci. Reports (2021), each of which is herein incorporated by reference in its entirety.

Plasmonic heating caused by illumination of a metal nanoparticle may facilitate dissociation of a binding reagent containing the metal nanoparticle from an analyte. For metal nanoparticles, such as noble metal nanoparticles, light wavelengths in the infrared (IR) range (e.g., near-IR, mid-IR, far-IR) may be useful for facilitating dissociation. In some cases, a metal nanoparticle may be illuminated with a light field containing light having a wavelength of between about 700 nanometer (nm) and about 1300 nm. In some cases, a metal nanoparticle may be illuminated with a light field containing light having a wavelength of between about 1300 nanometer (nm) and about 5600 nm. In some cases, a metal nanoparticle may be illuminated with a light field containing light having a wavelength of greater than 5600 nm. In some cases, a metal nanoparticle may be illuminated with a light field containing light having a wavelength of at least about 700 nm, 1000 nm, 1200 nm, 1400 nm, 1600 nm, 1800 nm, 2000 nm, 2200 nm, 2400 nm, 2600 nm, 2800 nm, 3000 nm, 3200 nm, 3400 nm, 3600 nm, 3800 nm, 4000 nm, 4200 nm, 4400 nm, 4600 nm, 4800 nm, 5000 nm, or more than 5000 nm. In some cases, a metal nanoparticle may be illuminated with a light field containing light having a wavelength of no more than about 5000 nm, 4800 nm, 4600 nm, 4400 nm, 4200 nm, 4000 nm, 3800 nm, 3600 nm, 3400 nm, 3200 nm, 3000 nm, 2800 nm, 2600 nm, 2400 nm, 2200 nm, 2000 nm, 1800 nm, 1600 nm, 1400 nm, 1200 nm, 1000 nm, 700 nm, or less than 700 nm.

It may be useful to provide affinity reagents comprising a metal nanoparticle, in which the metal nanoparticle is configured to emit a fluorescent signal, and in which the metal nanoparticle is further configured to produce heat by a thermoplasmonic effect. Such particles are described in, for example, Karan, N. S., et al. “Plasmonic Giant Quantum Dots: Hybrid Nanostructures for Truly Simultaneous Imaging, Photothermal Effect, and Thermometry.” Chem. Sci. (2015), which is herein incorporated by reference in its entirety. In some cases, a metal nanoparticle may comprise a quantum dot or other fluorescent metal nanoparticle. A metal nanoparticle may further comprise a layer, coating or shell comprising a noble metal.

A method may comprise one or more steps of: a) providing an analyte immobilized on a solid support at a fixed address, b) coupling a binding reagent to the analyte at the fixed address, wherein the binding reagent comprises an affinity agent coupled to a metal nanoparticle, c) detecting a detectable signal from the binding reagent at the fixed address, and d) after detecting the detectable signal, contacting the metal nanoparticle with a light field comprising light with an infrared wavelength. In some cases, detecting the detectable signal from the binding reagent can comprise contacting the metal nanoparticle with a light field comprising with a visible wavelength.

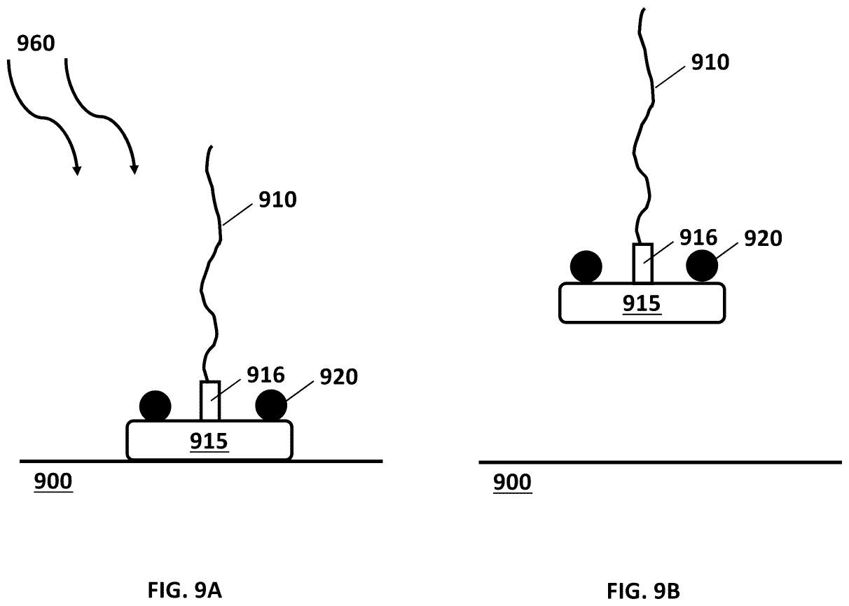

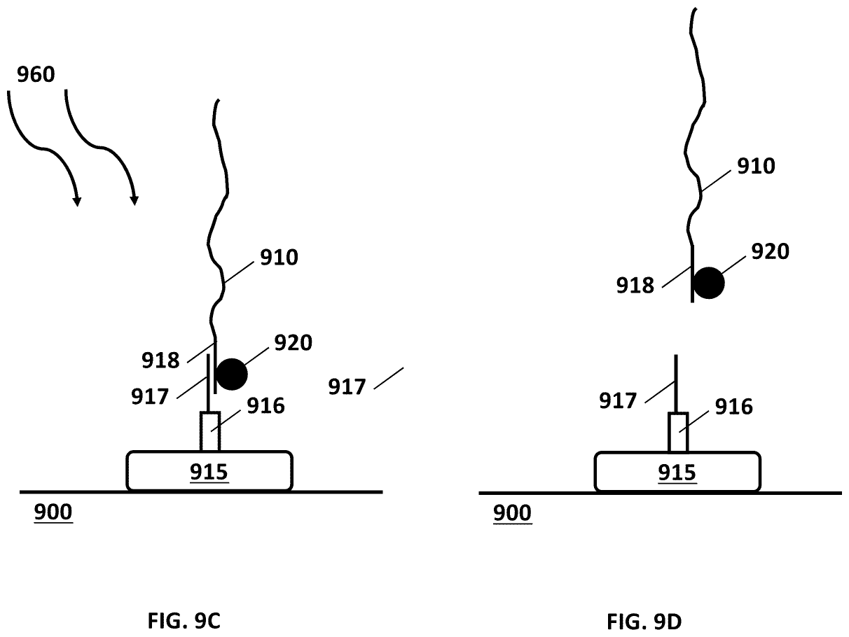

Thermoplasmonic heating may be useful for releasing array-bound entities, such as particles or analytes, from an array surface. In some cases, a method may involve selectively removing an array-bound entity from an address of an array. In some cases, the method may involve selectively removing an array-bound entity without releasing FIGS. 9A-9D illustrate aspects of releasing array-bound entities via thermoplasmonic heating of a metal nanoparticle attached to the array-bound entity. FIGS. 9A-9B illustrate a method of releasing a particle 915 (e.g., a nucleic acid nanoparticle) from a solid support 900 via thermoplasmonic heating of a metal nanoparticle 920. As shown in FIG. 9A, the particle 915 is attached to an analyte 910 by an optional linking moiety 916. The particle 915 may be attached to the surface of the solid support 900 by a covalent or non-covalent attachment, as set forth herein. The particle is attached to one or more metal nanoparticles 920. The address containing the particle 915 and/or its metal nanoparticle(s) 920 is illuminated by a light field 960, preferably containing light within the infrared region of the electromagnetic spectrum. The light field 960 induces localized heating due to thermoplasmonic heating of the one or more metal nanoparticles 920. FIG. 9B illustrates a second configuration, in which the particle 915 has released from the solid support 900. Optionally, the method may further comprise rinsing the mobile particle 915 from the solid support 900 with a fluidic medium.

FIGS. 9C-9D illustrate a method of releasing an analyte 910 from a solid support 900 via thermoplasmonic heating of a metal nanoparticle 920. FIG. 9C illustrates an analyte 910 attached to an optional particle 915 by a linking moiety 916. The particle 915 attaches the analyte 910 to the solid support 900. The linking moiety 916 comprises an oligonucleotide 917 that attached to a complementary oligonucleotide 918 that is attached to the analyte 910. The skilled person will readily recognize numerous additional configurations of covalent or non-covalent attachment of the analyte 910 to the particle 915. The complementary oligonucleotide 918 is attached to a metal nanoparticle. The address containing the complementary oligonucleotide 919 and/or its metal nanoparticle(s) 920 is illuminated by a light field 960, preferably containing light within the infrared region of the electromagnetic spectrum. The light field 960 induces localized heating due to thermoplasmonic heating of the one or more metal nanoparticles 920. FIG. 9D illustrates a second configuration, in which the analyte 910 has released from the particle 915 and the solid support 900. Optionally, the method may further comprise rinsing the mobile analyte 910 from the solid support 900 with a fluidic medium.

An array-bound entity may be released from an array by a method set forth herein for several reasons. In some cases, an array may be provided with an address containing a particle that is not attached to an analyte. A method may comprise releasing a particle that is not attached to an analyte. In some cases, an array may be provided with an address containing two or more particles, wherein each particle of the two or more particles is attached to an individual analyte. A method may comprise releasing at least one array-bound entity (e.g., a particle, an analyte, a particle-analyte complex) from an address containing two or more array-bound entities. A method may comprise releasing all but one array-bound entity from an address containing two or more array-bound entities. In some cases, an array may be provided with an entity (e.g., an affinity agent, a polypeptide, a nucleic acid, etc.) bound (e.g., irreversibly bound, reversibly bound) at an array address (e.g., bound to an analyte, bound to a particle, bound to a solid support). For example, a binding reagent may become irreversibly bound to an analyte due to a photo-catalyzed reaction). In another example, a binding reagent may bind non-specifically to an array site or a particle attached thereto. A method may comprise releasing an entity bound to an array address.

In some cases, a method may comprise illuminating a single array address with a light field. In other cases, a method may comprise illuminating a plurality of array addresses with a light field. Light may be provided to one or more array addresses by any suitable method of forming a localized light field, such as structured illumination or raster scanning. For illumination of a single light field, a characteristic dimension of a light field (e.g., length, width, diameter) may be less than an average spacing between an array site and its nearest neighbor site (i.e., average array site pitch) at its point of impingement on the array site. For example, an array with an average inter-site spacing of 1 micron may be contacted with a light field having a 500 nm diameter. Accordingly, only a single array site can be illuminated by the light field at any time. Alternatively, a characteristic dimension of a light field (e.g., length, width, diameter) may be greater than an average spacing between an array site and its nearest neighbor site (i.e., average array site pitch) at its point of impingement on the array site. Accordingly, only a plurality of sites can be illuminated by the light field at any time.

### Single-Analyte Assays

The present disclosure provides compositions, apparatus and methods for detecting one or more proteins. A protein can be detected using one or more affinity agents having binding affinity for the protein. The affinity agent and the protein can bind each other to form a complex and, during or after formation, the complex can be detected. The complex can be detected directly, for example, due to a label that is present on the affinity agent or protein. In some configurations, the complex need not be directly detected. For example, complex formation can yield a chemical change, such as formation of a nucleic acid tag, that is detected after the complex has been formed and in some cases after the complex has been dissociated.

Many protein detection methods, such as enzyme linked immunosorbent assay (ELISA), achieve high-confidence characterization of one or more protein in a sample by exploiting high specificity binding of antibodies, aptamers or other binding agents to the protein(s). ELISA is generally carried out at low plex scale (e.g. from one to a hundred different proteins detected in parallel or in succession) but can be used at higher plexity is some situations for which specialized hardware or reagents are available. ELISA methods can be carried out by detecting immobilized binding agents and/or proteins in multiwell plates, on arrays, or on particles in microfluidic devices. Exemplary plate-based methods include, for example, the MULTI-ARRAY technology commercialized by MesoScale Diagnostics (Rockville, Maryland) or Simple Plex technology commercialized by Protein Simple (San Jose, CA). Exemplary, array-based methods include, but are not limited to those utilizing Simoa® Planar Array Technology or Simoa® Bead Technology, commercialized by Quanterix (Billerica, MA). Further exemplary array-based methods are set forth in U.S. Pat. Nos. 9,678,068; 9,395,359; 8,415,171; 8,236,574; or 8,222,047, each of which is incorporated herein by reference. Exemplary microfluidic detection methods include those commercialized by Luminex (Austin, Texas) under the trade name xMAP® technology or used on platforms identified as MAGPIX®, LUMINEX® 100/200 or FEXMAP 3D®.

Other detection methods that can also be used, for example at low plex scale, include procedures that employ SOMAmer reagents and SOMAscan assays commercialized by Soma Logic (Boulder, CO). In one configuration, a sample is contacted with aptamers that are capable of binding proteins with specificity for the amino acid sequence of the proteins. The resulting aptamer-protein complexes can be separated from other sample components, for example, by attaching the complexes to beads (or other solid support) that are removed from other sample components. The aptamers can then be isolated and, because the aptamers are nucleic acids, the aptamers can be detected using any of a variety of methods known in the art for detecting nucleic acids, including for example, hybridization to nucleic acid arrays, PCR-based detection, or nucleic acid sequencing. Exemplary methods and compositions are set forth in U.S. Pat. Nos. 7,855,054; 7,964,356; 8,404,830; 8,945,830; 8,975,026; 8,975,388; 9,163,056; 9,938,314; 9,404,919; 9,926,566; 10,221,421; 10,239,908; 10,316,321 10,221,207 or 10,392,621, each of which is incorporated herein by reference.

The present disclosure provides compositions, apparatus and methods that can be useful for characterizing analytes, such as proteins, by obtaining multiple separate and non-identical measurements of the analytes. In particular configurations, the individual measurements may not, by themselves, be sufficiently accurate or specific to make the characterization, but in combination the multiple non-identical measurements can allow the characterization to be made with a high degree of accuracy, specificity and confidence. For example, the multiple separate measurements can include subjecting a sample to reagents that are promiscuous with regard to recognizing a variety of different analytes that are present in the sample. Accordingly, a first measurement carried out using a first promiscuous reagent may perceive a first subset of the analytes without distinguishing different analytes within the subset. A second measurement carried out using a second promiscuous reagent may perceive a second subset of analytes, again, without distinguishing one analyte in the second subset from other analytes in the second subset. However, a comparison of the first and second measurements can distinguish: (i) an analyte that is uniquely present in the first subset but not the second; (ii) an analyte that is uniquely present in the second subset but not the first; (iii) an analyte that is uniquely present in both the first and second subsets; or (iv) an analyte that is uniquely absent in the first and second subsets. The number of promiscuous reagents used, the number of separate measurements acquired, and degree of reagent promiscuity (e.g. the diversity of components recognized by the reagent) can be adjusted to suit the diversity of analytes expected for a particular sample.

The present disclosure provides assays that are useful for detecting one or more analytes. Exemplary assays are set forth herein in the context of detecting proteins. Those skilled in the art will recognize that methods, compositions and apparatus set forth herein can be adapted for use with other analytes such as cells, organelles, nucleic acids, polysaccharides, metabolites, vitamins, hormones, enzyme co-factors, therapeutic agents, candidate therapeutic agents and others set forth herein or known in the art. Particular configurations of the methods, apparatus and compositions set forth herein can be made and used, for example, as set forth in US Pat. Nos. 10,473,654 or 11,282,585; US Pat. App. Pub. Nos. 2020/0082914A1 or 2023/0114905A1; or Egertson et al., BioRxiv (2021), DOI: 10.1101/2021.10.11.463967, each of which is incorporated herein by reference. Exemplary methods, systems and compositions are set forth in further detail below.

A composition, apparatus or method set forth herein can be used to characterize an analyte, or moiety thereof, with respect to any of a variety of characteristics or features including, for example, presence, absence, quantity (e.g. amount or concentration), chemical reactivity, molecular structure, structural integrity (e.g. full length or fragmented), maturation state (e.g. presence or absence of pre- or pro-sequence in a protein), location (e.g. in an analytical system, subcellular compartment, cell or natural environment), association with another analyte or moiety, binding affinity for another analyte or moiety, biological activity, chemical activity or the like. An analyte can be characterized with regard to a relatively generic characteristic such as the presence or absence of a common structural feature (e.g. amino acid sequence length, overall charge or overall pKa for a protein) or common moiety (e.g. a short primary sequence motif or post-translational modification for a protein). An analyte can be characterized with regard to a relatively specific characteristic such as a unique amino acid sequence (e.g. for the full length of the protein or a motif), an RNA or DNA sequence that encodes a protein (e.g. for the full length of the protein or a motif), or an enzymatic or other activity that identifies a protein. A characterization can be sufficiently specific to identify an analyte, for example, at a level that is considered adequate or unambiguous by those skilled in the art.

In particular configurations, a method set forth herein can be used to identify a number of different extant proteins that exceeds the number of affinity reagents used. For example, the number of different protein species identified can be at least 5×, 10×, 25×, 50×, 100× or more than the number of affinity reagents used. This can be achieved, for example, by (1) using promiscuous affinity reagents that bind to multiple different candidate proteins suspected of being present in a given sample, and (2) subjecting the extant proteins to a set of promiscuous affinity reagents that, taken as a whole, are expected to bind each candidate protein in a different combination, such that each candidate protein is expected to generate a unique profile of binding and non-binding events when subjected to the set. Promiscuity of an affinity reagent can arise due to the affinity reagent recognizing an epitope that is known to be present in a plurality of different candidate proteins. For example, epitopes having relatively short amino acid lengths such as dimers, trimers, tetramers or pentamers are expected to occur in a substantial number of different proteins in a typical proteome. Alternatively or additionally, a given promiscuous affinity reagent may recognize multiple different epitopes (e.g. epitopes differing from each other with regard to amino acid composition or sequence). For example, a promiscuous affinity reagent that is designed or selected for its affinity toward a first trimer epitope may also have affinity for a second epitope that has a different sequence of amino acids compared to the first epitope.

Although performing a single binding reaction between a promiscuous affinity reagent and a complex protein sample may yield ambiguous results regarding the identity of the different extant proteins to which it binds, the ambiguity can be resolved by decoding the binding profiles for each extant protein using machine learning or artificial intelligence algorithms that are based on probabilities for the affinity reagents binding to candidate proteins. For example, a plurality of different promiscuous affinity reagents can be contacted with a complex population of extant proteins, wherein the plurality is configured to produce a different binding profile for each candidate protein suspected of being present in the population. The plurality of promiscuous affinity reagents can produce a binding profile for each extant protein that can be decoded to identify a unique combination of positive outcomes (i.e. observed binding events) and/or negative binding outcomes (i.e. observed non-binding events), and this can in turn be used to identify the extant protein as a particular candidate protein having a high likelihood of exhibiting a similar binding profile.

Binding profiles can be obtained for extant proteins and the binding profiles can be decoded or disambiguated to identify extant proteins corresponding to the binding profiles. In many cases one or more binding events produces inconclusive or even aberrant results and this, in turn, can yield ambiguous binding profiles. For example, observation of binding outcomes at single-molecule resolution can be particularly prone to ambiguities due to stochasticity in the behavior of single molecules when observed using certain detection hardware. As set forth above, ambiguity can also arise from affinity reagent promiscuity. Decoding can utilize a binding model that evaluates the likelihood or probability that one or more candidate proteins that are suspected of being present in an assay will have produced an empirically observed binding profile. The binding model can include information regarding expected binding outcomes (e.g. positive binding outcomes and/or negative binding outcomes) for one or more affinity reagents with respect to one or more candidate proteins. A binding model can include a measure of the probability or likelihood of a given candidate protein generating a false positive or false negative binding result in the presence of a particular affinity reagent, and such information can optionally be included for a plurality of affinity reagents.

Decoding can be configured to evaluate the degree of compatibility of one or more empirical binding profiles with results computed for various candidate proteins using a binding model. For example, to identify an extant protein in a sample, an empirical binding profile for the extant protein can be compared to results computed by the binding model for many or all candidate proteins suspected to be in the sample. A machine learning or artificial intelligence algorithm can be used. An algorithm used for decoding can utilize Bayesian inference. In some configurations, identity for an extant protein is determined based on a likelihood of the extant protein being a particular candidate protein given the empirical binding pattern or based on the probability of a particular candidate protein generating the empirical binding pattern. Particularly useful decoding methods are set forth, for example, in US Pat. Nos. 10,473,654 or 11,282,585; US Pat. App. Pub. Nos. 2020/0082914A1 or 2023/0114905A1; or Egertson et al., BioRxiv (2021), DOI: 10.1101/2021.10.11.463967, each of which is incorporated herein by reference. It will be recognized that methods set forth herein that are utilized to decode extant proteins may be useful for other analyte identification assays, provided said analyte identification assays provide a binding profile that can be decoded.

In some detection assays, a protein can be cyclically modified and the modified products from individual cycles can be detected. For example, a protein can be sequenced by a sequential process in which each cycle includes steps of detecting the protein and removing one or more terminal amino acids from the protein to produce a shortened protein. The shortened protein is then subjected to subsequent cycles. Optionally, a protein sequencing method can include steps of adding a label to the protein, for example, at the amino terminal amino acid or at the carboxy terminal amino acid. In particular configurations, a method a protein sequencing method can include steps of (i) removing a terminal amino acid from the protein, thereby forming a truncated protein; (ii) detecting a change in signal from the truncated protein, for example, in comparison to the protein prior to truncation; and (iii) identifying the type of amino acid that was removed in step (i) based on the change detected in step (ii). The terminal amino acid can be removed, for example, by removal of one or more amino acids from the amino terminus or carboxyl terminus of the protein. Steps (i) through (iii) can be repeated to produce a series of signal changes that is indicative of the sequence for the protein.

In a first configuration of a protein sequencing method, one or more types of amino acids in the protein can be attached to a label that uniquely identifies the type of amino acid. In this configuration, the change in signal that identifies the amino acid can be loss of signal from the respective label. For example, lysines can be attached to a distinguishable label such that loss of the label indicates removal of a lysine. Alternatively or additionally, other amino acid types can be attached to other labels that are mutually distinguishable from lysine and from each other. For example, lysines can be attached to a first label and cysteines can be attached to a second label, the first and second labels being distinguishable from each other. Exemplary compositions and techniques that can be used to remove amino acids from a protein and detect signal changes are those set forth in Swaminathan et al., Nature Biotech. 36:1076-1082 (2018); or U.S. Pat. No. 9,625,469 or U.S. Pat. No. 10,545,153, each of which is incorporated herein by reference. Methods and apparatus under development by Erisyon, Inc. (Austin, TX) may also be useful for sequencing, or otherwise detecting, proteins.

In a second configuration of a cyclical protein detection method, a terminal amino acid of a protein can be recognized by an affinity agent that is specific for the terminal amino acid, specific for a labeled terminal amino acid (e.g. the affinity agent can recognize the label alone or in combination with the side chain of a particular type of amino acid). The affinity agent can be detected on the array, for example, due to a label on the affinity agent. Optionally, the label is a nucleic acid barcode sequence that is added to a primer nucleic acid upon formation of a complex. For example, a barcode can be added to the primer via ligation of an oligonucleotide having the barcode sequence or polymerase extension directed by a template that encodes the barcode sequence. The formation of the complex and identity of the terminal amino acid can be determined by decoding the barcode sequence. Multiple cycles can produce a series of barcodes that can be detected, for example, using a nucleic acid sequencing technique. Exemplary affinity agents and detection methods are set forth in US Pat. App. Pub. No. 2019/0145982 A1; 2020/0348308 A1; or 2020/0348307 A1, each of which is incorporated herein by reference. Methods and apparatus under development by Encodia, Inc. (San Diego, CA) or Standard BioTools (e.g. technology developed by SomaLogic or Palamaedrix) may also be useful for detecting proteins.

Cyclical removal of terminal amino acids from a protein can be carried out using an Edman-type sequencing reaction. In some configurations, an Edman-type sequencing reaction can involve reaction of a phenyl isothiocyanate with an N-terminal amino group of a protein under mildly alkaline conditions (e.g. about pH 8) to form a cyclical phenylthiocarbamoyl Edman complex derivative. The phenyl isothiocyanate may be substituted or unsubstituted with one or more functional groups, linker groups, or linker groups containing functional groups. An Edman-type sequencing reaction can include variations to reagents and conditions that yield detectable removal of amino acids from a protein terminus, thereby facilitating determination of the amino acid sequence for a protein or portion thereof. For example, the phenyl group can be replaced with at least one aromatic, heteroaromatic or aliphatic group which may participate in an Edman-type sequencing reaction, non-limiting examples including: pyridine, pyrimidine, pyrazine, pyridazoline, fused aromatic groups such as naphthalene and quinoline), methyl or other alkyl groups or alkyl group derivatives (e.g., alkenyl, alkynyl, cyclo-alkyl). Under certain conditions, for example, acidic conditions of about pH 2, derivatized terminal amino acids may be cleaved, for example, as a thiazolinone derivative. The thiazolinone amino acid derivative under acidic conditions may form a more stable phenylthiohydantoin (PTH) or similar amino acid derivative which can be detected. This procedure can be repeated iteratively for residual protein to identify the subsequent N-terminal amino acid. Many variations of Edman-type degradation have been described and may be used including, for example, a one-step removal of an N-terminal amino acid using alkaline conditions (Chang, J. Y., FEBS LETTS., 1978, 91(1), 63-68). In some cases, Edman-type reactions may be thwarted by N-terminal modifications which may be selectively removed, for example, N-terminal acetylation or formylation (e.g., see Gheorghe M. T., Bergman T. (1995) in Methods in Protein Structure Analysis, Chapter 8: Deacetylation and internal cleavage of Proteins for N-terminal Sequence Analysis. Springer, Boston, MA. https://doi.org/10.1007/978-1-4899-1031-8_8).

Non-limiting examples of functional groups for substituted phenyl isothiocyanate may include ligands (e.g. biotin and biotin analogs) for known receptors, labels such as luminophores, or reactive groups such as click functionalities (e.g. compositions having an azide or acetylene moiety). The functional group may be a DNA, RNA, peptide or small molecule barcode or other tag which may be further processed and/or detected.

Edman-type processes can be carried out in a multiplex format to detect, characterize or identify a plurality of proteins. A method of detecting a protein can include steps of (i) exposing a terminal amino acid on a protein at an address of an array; (ii) binding an affinity agent to the terminal amino acid, where the affinity agent includes a nucleic acid tag, and where a primer nucleic acid is present at the address; (iii) extending the primer nucleic acid in the presence of the nucleic acid tag, thereby producing an extended primer having a copy of the tag; and (iv) detecting the tag of the extended primer. The terminal amino acid can be exposed, for example, by removal of one or more amino acids from the amino terminus or carboxyl terminus of the protein. Steps (i) through (iv) can be repeated to produce a series of tags that is indicative of the sequence for the protein. The method can be applied to a plurality of proteins on the array and in parallel. The extending of a primer can be carried out, for example, by polymerase-based extension of the primer, using the nucleic acid tag as a template. Alternatively, the extending of a primer can be carried out, for example, by ligase- or chemical-based ligation of the primer to a nucleic acid that is hybridized to the nucleic acid tag. The nucleic acid tag can be detected via hybridization to nucleic acid probes (e.g. in an array), amplification-based detections (e.g. PCR-based detection, or rolling circle amplification-based detection) or nuclei acid sequencing (e.g. cyclical reversible terminator methods, nanopore methods, or single molecule, real time detection methods). Exemplary methods that can be used for detecting proteins using nucleic acid tags are set forth in US Pat. App. Pub. No. 2019/0145982 A1; 2020/0348308 A1; or 2020/0348307 A1, each of which is incorporated herein by reference.

A protein can optionally be detected based on its enzymatic or biological activity. For example, a protein can be contacted with a reactant that is converted to a detectable product by an enzymatic activity of the protein. In other assay formats, a first protein having a known enzymatic function can be contacted with a second protein to determine if the second protein changes the enzymatic function of the first protein. As such, the first protein serves as a reporter system for detection of the second protein. Exemplary changes that can be observed include, but are not limited to, activation of the enzymatic function, inhibition of the enzymatic function, attenuation of the enzymatic function, degradation of the first protein or competition for a reactant or cofactor used by the first protein. Proteins can also be detected based on their binding interactions with other molecules such as other proteins, nucleic acids, nucleotides, metabolites, hormones, vitamins, small molecules that participate in biological signal transduction pathways, biological receptors or the like. For example, a protein that participates in a signal transduction pathway can be identified as a particular candidate protein by detecting binding to a second protein that is known to be a binding partner for the candidate protein in the pathway.

In some configurations of the apparatus and methods set forth herein, one or more proteins can be detected on a solid support. For example, protein(s) can be attached to a solid support, the solid support can be contacted with detection agents (e.g. affinity agents) in solution, the agents can interact with the protein(s), thereby producing a detectable signal, and then the signal can be detected to determine the presence of the protein(s). In multiplexed versions of this approach, different proteins can be attached to different addresses in an array, and the probing and detection steps can occur in parallel. In another example, affinity agents can be attached to a solid support, the support can be contacted with proteins in solution, the proteins can interact with the affinity agents, thereby producing a detectable signal, and then the signal can be detected to determine presence, quantity or characteristics of the proteins. This approach can also be multiplexed by attaching different affinity agents to different addresses of an array.

Proteins, affinity agents or other objects of interest can be attached to a solid support via covalent or non-covalent bonds. For example, a linker can be used to covalently attach a protein or other object of interest to an array. A particularly useful linker is a structured nucleic acid particle such as a nucleic acid nanoball (e.g. a concatemeric amplicon produced by rolling circle replication of a circular nucleic acid template) or a nucleic acid origami. For example, a plurality of proteins can be conjugated to a plurality of structured nucleic acid particles, such that each protein-conjugated particle forms a respective address in the array. Exemplary linkers for attaching proteins, or other objects of interest, to an array or other solid support are set forth in US Pat. Nos. 11,203,612 or 11,505,796 or US Pat. App. Pub. No. 2023/0167488 A1, each of which is incorporated herein by reference.

A protein can be detected based on proximity of two or more affinity agents. For example, the two affinity agents can include two components each: a receptor component and a nucleic acid component. When the affinity agents bind in proximity to each other, for example, due to ligands for the respective receptors being at the same address in an array, the nucleic acids can interact to cause a modification that is indicative of the two ligands being in proximity. Optionally, the modification can be polymerase catalyzed extension of one of the nucleic acids using the other nucleic acid as a template. As another option, one of the nucleic acids can form a template that acts as splint to position other nucleic acids for ligation to an oligonucleotide. Exemplary methods are commercialized by Olink Proteomics AB (Uppsala Sweden) or set forth in U.S. Pat. Nos. 7,306,904; 7,351,528; 8,013,134; 8,268,554 or 9,777,315, each of which is incorporated herein by reference.

In some configurations of the compositions, apparatus and methods set forth herein, one or more proteins can be present on a solid support, where the proteins can optionally be detected. For example, a protein can be attached to a solid support, the solid support can be contacted with a detection agent (e.g. affinity agent) in solution, the affinity agent can interact with the protein, thereby producing a detectable signal, and then the signal can be detected to determine the presence, absence, quantity, a characteristic or identity of the protein. In multiplexed versions of this approach, different proteins can be attached to different addresses in an array, and the detection steps can occur in parallel, such that proteins at each address are detected, quantified, characterized or identified. In another example, detection agents can be attached to a solid support, the support can be contacted with proteins in solution, the proteins can interact with the detection agents, thereby producing a detectable signal, and then the signal can be detected to determine the presence of the proteins. This approach can also be multiplexed by attaching different probes to different addresses of an array.

In multiplexed configurations, different proteins can be attached to different unique identifiers (e.g. addresses in an array), and the proteins can be manipulated and detected in parallel. For example, a fluid containing one or more different affinity agents can be delivered to an array such that the proteins of the array are in simultaneous contact with the affinity agent(s). Moreover, a plurality of addresses can be observed in parallel allowing for rapid detection of binding events. A plurality of different proteins can have a complexity of at least 5, 10, 100, 1×10³, 1×10⁴, 1×10⁵ or more different native-length protein primary sequences. Alternatively or additionally, a proteome, proteome subfraction or other protein sample that is analyzed in a method set forth herein can have a complexity that is at most 1×10⁵, 1×10⁴, 1×103, 100, 10, 5 or fewer different native-length protein primary sequences. The total number of proteins of a sample that is detected, characterized or identified can differ from the number of different primary sequences in the sample, for example, due to the presence of multiple copies of at least some protein species. Moreover, the total number of proteins of a sample that is detected, characterized or identified can differ from the number of candidate proteins suspected of being in the sample, for example, due to the presence of multiple copies of at least some protein species, absence of some proteins in a source for the sample, or loss of some proteins prior to analysis.

A protein can be attached to a unique identifier using any of a variety of means. The attachment can be covalent or non-covalent. Exemplary covalent attachments include chemical linkers such as those achieved using click chemistry or other linkages known in the art or described in U.S. patent application Ser. No. 17/062,405, which is incorporated herein by reference. Non-covalent attachment can be mediated by receptor-ligand interactions (e.g. (strept) avidin-biotin, antibody-antigen, or complementary nucleic acid strands), for example, wherein the receptor is attached to the unique identifier and the ligand is attached to the protein or vice versa. In particular configurations, a protein is attached to a solid support (e.g. an address in an array) via a structured nucleic acid particle (SNAP). A protein can be attached to a SNAP and the SNAP can interact with a solid support, for example, by non-covalent interactions of the DNA with the support and/or via covalent linkage of the SNAP to the support. Nucleic acid origami or nucleic acid nanoballs are particularly useful. The use of SNAPs and other moieties to attach proteins to unique identifiers such as tags or addresses in an array are set forth in U.S. patents application Ser. Nos. 17/062,405 and 63/159,500, each of which is incorporated herein by reference.

A method set forth herein can be carried out in a fluid phase or on a solid phase. For fluid phase configurations, a fluid containing one or more proteins can be mixed with another fluid containing one or more affinity agents. For solid phase configurations one or more proteins or affinity agents can be attached to a solid support. One or more components that will participate in a binding event can be contained in a fluid and the fluid can be delivered to a solid support, the solid support being attached to one or more other component that will participate in the binding event. A solid support can be composed of a substrate that is insoluble in aqueous liquid. The substrate can have any of a variety of other characteristics such as being rigid, non-porous or porous. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, inorganic glasses, optical fiber bundles, gels, and polymers. In some cases, a solid support may comprise silicon, fused silica, quartz, mica, or borosilicate glass. In particular configurations a flow cell contains the solid support such that fluids introduced to the flow cell can interact with a surface of the solid support to which one or more components of a binding event (or other reaction) is attached.

A method of the present disclosure can be carried out at single analyte resolution. As such, a single analyte (i.e. one and only one analyte), such as a single protein, can be individually manipulated or distinguished using a method set forth herein. A single analyte can be a single molecule (e.g. single protein), a single complex of two or more molecules (e.g. a single protein attached to a structured nucleic acid particle or a single protein attached to an affinity agent), a single particle, or the like. A single analyte may be resolved from other analytes based on, for example, spatial or temporal separation from the other analytes. Reference herein to a ‘single analyte’ in the context of a composition, apparatus or method does not necessarily exclude application of the composition, apparatus or method to multiple single analytes that are manipulated or distinguished individually, unless indicated to the contrary.

Alternatively to single-analyte resolution, a method can be carried out at ensemble-resolution or bulk-resolution. Bulk-resolution configurations acquire a composite signal from a plurality of different analytes or affinity agents in a vessel or on a surface. For example, a composite signal can be acquired from a population of different protein-affinity agent complexes in a well or cuvette, or on a solid support surface, such that individual complexes are not resolved from each other. Ensemble-resolution configurations acquire a composite signal from a first collection of proteins or affinity agents in a sample, such that the composite signal is distinguishable from signals generated by a second collection of proteins or affinity agents in the sample. For example, the ensembles can be located at different addresses in an array. Accordingly, the composite signal obtained from each address will be an average of signals from the ensemble, yet signals from different addresses can be distinguished from each other.

A composition, apparatus or method set forth herein can be configured to contact one or more analytes (e.g. an array of different proteins) with a plurality of different affinity agents. For example, a plurality of affinity agents (whether configured separately or as a pool) may include at least 2, 5, 10, 25, 50, 100, 250, 500 or more types of affinity agents, each type of affinity agent differing from the other types with respect to the epitope(s) recognized. Alternatively or additionally, a plurality of affinity agents may include at most 500, 250, 100, 50, 25, 10, 5, or 2 types of affinity agents, each type of affinity agent differing from the other types with respect to the epitope(s) recognized. Different types of affinity agents in a pool can be uniquely labeled such that the different types can be distinguished from each other. In some configurations, at least two, and up to all, of the different types of affinity agents in a pool may be indistinguishably labeled with respect to each other. Alternatively or additionally to the use of unique labels, different types of affinity agents can be delivered and detected serially when evaluating one or more proteins (e.g. in an array).

A method of the present disclosure can be performed in a multiplex format. In multiplexed configurations, different analytes can be attached to different unique identifiers (e.g. proteins can be attached to different addresses in an array). Multiplexed analytes can be manipulated and detected in parallel. For example, a fluid containing one or more different affinity agents can be delivered to a protein array such that the proteins of the array are in simultaneous contact with the affinity agent(s). Moreover, a plurality of addresses can be observed in parallel allowing for rapid detection of binding events. A plurality of different proteins can have a complexity of at least 5, 10, 100, 1×10³, 1×10⁴, 2×10⁴, 3×10⁴ or more different native-length protein primary sequences. Alternatively or additionally, a plurality of different proteins can have a complexity that is at most 3×10⁴, 2×10⁴, 1×10⁴, 1×10³, 100, 10, 5 or fewer different native-length protein primary sequences. The plurality of proteins can constitute a proteome or subfraction of a proteome. The total number of proteins that is detected, characterized or identified can differ from the number of different primary sequences in the sample from which the proteins are derived, for example, due to the presence of multiple copies of at least some protein species. Moreover, the total number of proteins that are detected, characterized or identified can differ from the number of candidate proteins suspected of being present, for example, due to the presence of multiple copies of at least some protein species, absence of some proteins in a source for the proteins, or loss of some proteins prior to analysis.

A particularly useful multiplex format uses an array of analytes (e.g. proteins) and/or affinity agents. The analytes and/or affinity agents can be attached to unique identifiers (e.g. addresses of the array) such that the analytes can be distinguished from each other. An array can be used in any of a variety of processes such as an analytical process used for detecting, identifying, characterizing or quantifying an analyte. Analytes can be attached to unique identifiers via covalent or non-covalent (e.g. ionic bond, hydrogen bond, van der Waals forces etc.) bonds. An array can include different analyte species that are each attached to different unique identifiers. An array can include different unique identifiers that are attached to the same or similar analyte species. An array can include separate solid supports or separate addresses that each bear a different analyte, in which the different analytes can be identified according to the locations of the solid supports or addresses.

An address of an array can contain a single analyte, or it can contain a population of several analytes of the same species (i.e. an ensemble of the analytes). Alternatively, an address can include a population of different analytes. Addresses are typically discrete in an array. Discrete addresses that neighbor each other can be contiguous, or they can be separated by interstitial spaces. An array useful herein can have, for example, addresses that are separated by an average distance of less than 100 microns, 10 microns, 1 micron, 100 nm, 10 nm or less. Alternatively or additionally, an array can have addresses that are separated by an average distance of at least 10 nm, 100 nm, 1 micron, 10 microns, 100 microns or more. The addresses can each have an area of less than 1 square millimeter, 500 square microns, 100 square microns, 10 square microns, 1 square micron, 100 square nm or less. An array can include at least about 1×10⁴, 1×10⁵, 1×10⁶, 1×10⁷, 1×10⁸, 1×10⁹, 1×10¹⁰, 1×10¹¹, 1×10¹², or more addresses.

A protein or other analyte can be attached to a unique identifier (e.g. an address in an array) using any of a variety of means. The attachment can be covalent or non-covalent. Exemplary covalent attachments include chemical linkers such as those achieved using click chemistry or other linkages known in the art or described in US Pat. Nos. 11,203,612 or 11,505,796 or US Pat. App. Pub. No 2023/0167488 A1, each of which is incorporated herein by reference. Non-covalent attachment can be mediated by receptor-ligand interactions (e.g. (strept) avidin-biotin, antibody-antigen, or complementary nucleic acid strands), for example, in which the receptor is attached to the unique identifier and the ligand is attached to the protein or vice versa. In particular configurations, a protein is attached to a solid support (e.g. an address in an array) via a structured nucleic acid particle (SNAP). A protein can be attached to a SNAP and the SNAP can interact with a solid support, for example, by non-covalent interactions of the DNA with the support and/or via covalent linkage of the SNAP to the support. Nucleic acid origami or nucleic acid nanoballs are particularly useful SNAPs. The use of SNAPs and other moieties to attach proteins to unique identifiers such as tags or addresses in an array are set forth in US Pat. Nos. 11,203,612 or 11,505,796 or US Pat. App. Pub. No 2023/0167488 A1, each of which is incorporated herein by reference.

One or more compositions set forth herein can be present in an apparatus or vessel. For example, a composition of the present disclosure can be present in a vessel, such as a flow cell. As a further option, the vessel can be engaged with a detection apparatus. The vessel can be permanently or temporarily engaged with the detection apparatus. A detection apparatus can be configured to detect contents of a vessel, for example, by acquiring signals arising from the vessel. For example, a detection apparatus can be configured to acquire optical signals through an optically transparent window of the vessel. Optionally, the detection apparatus can be configured for luminescence detection, for example, having an optical train that delivers radiation from an excitation source (e.g. a laser or lamp) then through a window of the vessel. The detection apparatus can further include a camera or other detector that acquires signals transmitted through the window of the vessel and through an optical train. Optionally excitation and emission can be transmitted through the same optical train; however, separate optical trains can also be useful.

A detection apparatus can include a light sensing device that is appropriate for detecting a characteristic set forth herein or known in the art. Particularly useful components of a light sensing device can include, but are not limited to, optical sub-systems or components used in nucleic acid sequencing systems. Examples of useful sub systems and components thereof are set forth in US Pat. App. Pub. No. 2010/0111768 A1 or U.S. Pat. Nos. 7,329,860; 8,951,781 or 9,193,996, each of which is incorporated herein by reference. Other useful light sensing devices and components thereof are described in U.S. Pat. Nos. 5,888,737; 6,175,002; 5,695,934; 6,140,489; or 5,863,722; or US Pat. Pub. Nos. 2007/007991 A1, 2009/0247414 A1, or 2010/0111768; or WO2007/123744, each of which is incorporated herein by reference. Light sensing devices and components that can be used to detect luminophores based on luminescence lifetime are described, for example, in U.S. Pat. Nos. 9,678,012; 9,921,157; 10,605,730; 10,712,274; 10,775,305; or 10,895,534, each of which is incorporated herein by reference.

For configurations that use optical detection (e.g. luminescent detection), one or more analytes (e.g. proteins) may be immobilized on a surface, and this surface may be observed by a microscope to detect any signal from the immobilized analytes. The microscope itself may include a digital camera or other luminescence detector configured to record, store, and analyze the data collected during the scan. A luminescence detector can further include an excitation source that is capable of irradiating analytes, for example, proteins at addresses on an array, at an appropriate wavelength. A luminescence detector of the present disclosure can be configured for epiluminescent detection, total internal reflection (TIR) detection, waveguide assisted excitation, or the like. Optical filters or other optical components can be present to tune the wavelength, polarization or other optical properties of excitation and/or emission radiation used by a luminescence detector.

A light sensing device may be based upon any suitable technology, and may be, for example, a charged coupled device (CCD) sensor that generates pixilated image data based upon photons impacting locations in the device. It will be understood that any of a variety of other light sensing devices may also be used including, but not limited to, a detector array configured for time delay integration (TDI) operation, a complementary metal oxide semiconductor (CMOS) detector, an avalanche photodiode (APD) detector, a Geiger-mode photon counter, a photomultiplier tube (PMT), charge injection device (CID) sensors, JOT image sensor (Quanta), or any other suitable detector. Light sensing devices can optionally be coupled with one or more excitation sources, for example, lasers, light emitting diodes (LEDs), arc lamps or other energy sources known in the art.

A light sensing device can be configured for single molecule resolution. For example, waveguides or optical confinements can be used to deliver excitation radiation to locations of a solid support where analytes are located. Zero-mode waveguides can be particularly useful, examples of which are set forth in U.S. Pat. Nos. 7,181,122, 7,302,146, or 7,313,308, each of which is incorporated herein by reference. Analytes can be confined to surface features that function as addresses and facilitate single molecule resolution. For example, analytes can be distributed into wells having nanometer dimensions such as those set forth in U.S. Pat. No. 7,122,482 or U.S. Pat. No. 8,765,359, or US Pat. App. Pub. No 2013/0116153 A1, each of which is incorporated herein by reference. The wells can be configured for selective excitation, for example, as set forth in U.S. Pat. No. 8,798,414 or 9,347,829, each of which is incorporated herein by reference. Analytes can be distributed to nanometer-scale posts, such as high aspect ratio posts which can optionally be dielectric pillars that extend through a metallic layer to improve detection of an analyte attached to the pillar. See, for example, U.S. Pat. Nos. 8,148,264, 9,410,887 or U.S. Pat. No. 9,987,609, each of which is incorporated herein by reference. Further examples of nanostructures that can be used to detect analytes are those that change state in response to the concentration of analytes such that the analytes can be quantitated as set forth in WO 2020/176793 A1, which is incorporated herein by reference.

A detection apparatus need not be configured for optical detection. For example, an electronic detector can be used for detection of protons or charged labels (see, for example, US Pat. App. Pub. Nos. 2009/0026082 A1; 2009/0127589 A1; 2010/0137143 A1; or 2010/0282617 A1, each of which is incorporated herein by reference in its entirety). A field effect transistor (FET) can be used to detect analytes or other entities, for example, based on proximity of a field disrupting moiety to the FET. The field disrupting moiety can be due to an extrinsic label attached to an analyte or affinity reagent, or the moiety can be intrinsic to the analyte or affinity agent being used. Surface plasmon resonance can be used to detect binding of analytes or affinity agents at or near a surface. Exemplary sensors and methods for attaching molecules to sensors are set forth in US Pat. App. Pub. Nos. 2017/0240962 A1; 2018/0051316 A1; 2018/0112265 A1; 2018/0155773 A1 or 2018/0305727 A1; or U.S. Pat. Nos. 9,164,053; 9,829,456; 10,036,064, each of which is incorporated herein by reference.

Luminescence lifetime can be detected using an integrated circuit having a photodetection region configured to receive incident photons and produce a plurality of charge carriers in response to the incident photons. The integrated circuit can include at least one charge carrier storage region and a charge carrier segregation structure configured to selectively direct charge carriers of the plurality of charge carriers directly into the charge carrier storage region based upon times at which the charge carriers are produced. See, for example, U.S. Pat. Nos. 9,606,058, 10,775,305, and 10,845,308, each of which is incorporated herein by reference. Optical sources that produce short optical pulses can be used for luminescence lifetime measurements. For example, a light source, such as a semiconductor laser or LED, can be driven with a bipolar waveform to generate optical pulses with FWHM durations as short as approximately 85 ps having suppressed tail emission. See, for example, in U.S. Pat. No. 10,605,730, which is incorporated herein by reference.

A detection apparatus can include a fluidics system, for example, configured for fluidic communication with a vessel, such as a flow cell. In some configurations, a detection apparatus can include one or more reservoirs containing affinity reagents or analytes that are delivered to a vessel. Optionally, a detection apparatus can be configured to include a waste receptacle to which waste from the vessel is collected. For example, a composition set forth herein can be delivered from the apparatus through an ingress of a flow cell and waste can be removed through an egress of the flow cell to the apparatus.

One or more compositions set forth herein can be provided in kit form including, if desired, a suitable packaging material. Optionally, one or more compositions can be provided as a solid, such as crystals or a lyophilized pellet. Accordingly, any combination of reagents or components that is useful in a method set forth herein can be included in a kit.

The packaging material included in a kit can include one or more physical structures used to house the contents of the kit. The packaging material can be constructed by well-known methods, preferably to provide a sterile, contaminant-free environment. The packaging materials employed herein can include, for example, those customarily utilized in affinity reagent systems. Exemplary packaging materials include, without limitation, glass, plastic, paper, foil, and the like, capable of holding within fixed limits a component useful in the methods of the present disclosure.

Packaging material or other components of a kit can include a kit label which identifies or describes a particular method set forth herein. For example, a kit label can indicate that the kit is useful for detecting a particular protein or proteome. In another example, a kit label can indicate that the kit is useful for a therapeutic or diagnostic purpose, or alternatively that it is for research use only.

Instructions for use of the packaged reagents or components are also typically included in a kit. The instructions for use can include a tangible expression describing the reagent or component concentration or at least one assay method parameter, such as the relative amounts of kit components and sample to be admixed, maintenance time periods for reagent/sample admixtures, temperature, buffer conditions, and the like.

In some cases, a kit can be configured as a cartridge or component of a cartridge. The cartridge can in turn be configured to be engaged with a detection apparatus. For example, the cartridge can be engaged with a detection apparatus such that contents of the cartridge are in fluidic communication with the detection apparatus or with a flow cell engaged with the detection apparatus. A cartridge can be engaged with a detection apparatus such that contents of the cartridge can be observed by the detection apparatus, for example, using an assay set forth herein.

A solid support or a surface thereof may be configured to display an analyte or a plurality of analytes. A solid support may contain one or more addresses in formed or prepared surfaces. Multiple addresses can be configured to form a pattern. In some cases, a solid support may contain one or more patterned, formed, or prepared surfaces that contain a plurality of addresses, with each address configured to display one or more analytes. Accordingly, an array as set forth herein may comprise a plurality of analytes coupled to a solid support or a surface thereof. In some configurations, a solid support or a surface thereof may be patterned or formed to produce an ordered or repeating pattern of addresses. The deposition of analytes on the repeating pattern of addresses may be controlled by interactions between the solid support and the analytes such as, for example, electrostatic interactions, magnetic interactions, hydrophobic interactions, hydrophilic interactions, covalent interactions, or non-covalent interactions. Accordingly, the coupling of an analyte at each address of an array may produce an array of analytes whose average spacing between analytes is relatively uniform, for example, being determined based upon the tolerance of the ordering or patterning of the solid support and the size of an analyte-binding region for each address. An ordered or patterned array of analytes may be characterized as having a regular geometry, such as a rectangular, triangular, polygonal, or annular grid. In other configurations, a solid support or a surface thereof may have a random or non-repeating pattern of addresses. The deposition of analytes on the random or non-repeating pattern may be controlled by interactions between the solid support and the analytes, or inter-analyte interactions such as, for example, steric repulsion, electrostatic repulsion, electrostatic attraction, magnetic repulsion, magnetic attraction, covalent interactions, or non-covalent interactions.

A solid support or a surface thereof may contain one or more structures or features. A structure or feature may comprise an elevation, profile, shape, geometry, or configuration that deviates from an average elevation, profile, shape, geometry, or configuration of a solid support or surface thereof. A structure or feature may be a raised structure or feature, such as a ridge, post, pillar, or pad, if the structure or feature extends above the average elevation of a surface of a solid support. A structure or feature may be a depressed structure, such as a channel, well, pore, or hole, if the structure or feature extends below the average elevation of a surface of a solid support. A structure or feature may be an intrinsic structure or feature of a substrate (i.e., arising due to the physical or chemical properties of the substrate, or a physical or chemical mechanism of formation), such as surface roughness structures, crystal structures, or porosity. A structure or feature may be formed by a method of processing a solid support. In some configurations, a solid support or a surface may be processed by a lithographic method to form one or more structures or features. A solid support or a surface thereof may be formed by a suitable lithographic method, including, but not limited to photolithography, Dip-Pen nanolithography, nanoimprint lithography, nanosphere lithography, nanoball lithography, nanopillar arrays, nanowire lithography, immersion lithography, neutral particle lithography, plasmonic lithography, scanning probe lithography, thermochemical lithography, thermal scanning probe lithography, local oxidation nanolithography, molecular self-assembly, stencil lithography, laser interference lithography, soft lithography, magnetolithography, stereolithography, deep ultraviolet lithography, x-ray lithography, ion projection lithography, proton-beam lithography, or electron-beam lithography.

A solid support or surface may comprise a plurality of structures or features. Structures or features may be provided as analyte-binding sites for the coupling of analytes or other moieties (e.g., anchoring moieties). A plurality of structures or features may comprise a repeating pattern of structures or features. A plurality of structures or features may comprise a non-ordered, non-repeating, or random distribution of structures or features. A structure or feature may have an average characteristic dimension (e.g., length, width, height, diameter, circumference, etc.) of at least about 1 nanometer (nm), 5 nm, 10 nm, 20 nm, 30 nm, 40 nm, 50 nm, 75 nm, 100 nm, 150 nm, 200 nm, 250 nm, 300 nm, 400 nm, 500 nm, 750 nm, 1000 nm, or more than 1000 nm. Alternatively or additionally, a structure or feature may have an average characteristic dimension of no more than about 1000 nm, 750 nm, 500 nm, 400 nm, 300 nm, 250 nm, 200 nm, 150 nm, 100 nm, 75 nm, 50 nm, 40 nm, 30 nm, 20 nm, 10 nm, 5 nm, 1 nm, or less than 1 nm. An array of structures or features may have an average pitch, in which the pitch is measured as the average separation between respective centerpoints of adjacent structures or features. An array may have an average pitch of at least about 1 nm, 5 nm, 10 nm, 20 nm, 30 nm, 40 nm, 50 nm, 75 nm, 100 nm, 150 nm, 200 nm, 250 nm, 300 nm, 400 nm, 500 nm, 750 nm, 1 micron (μm), 2 μm, 5 μm, 10 μm, 50 μm, 100 μm, or more than 100 μm. Alternatively or additionally, an array may have an average pitch of no more than about 100 μm, 50 μm, 10 μm, 5 μm, 1 μm, 750 nm, 500 nm, 400 nm, 300 nm, 250 nm, 200 nm, 150 nm, 100 nm, 75 nm, 50 nm, 40 nm, 30 nm, 20 nm, 10 nm, 5 nm, 1 nm, or less than 1 nm.

A structure or feature of an array may have a characteristic dimension (e.g., a width, length, or diameter) that is smaller than a characteristic dimension of an analyte or other object (e.g., a nanoparticle) that is attached to the structure or feature. It may be preferable to provide structures or features that are smaller than analytes or other objects attached to the structure or feature to occlude the attachment of additional analytes or other objects to the structure or feature. Alternatively, a structure or feature may have a characteristic dimension that is larger than a characteristic dimension of an analyte or other object (e.g., a nanoparticle) that is attached to the structure or feature.

A solid support or a surface thereof may include a base substrate material and, optionally, one or more additional materials that are contacted or adhered with the substrate material. A solid support may comprise one or more additional materials that are deposited, coated, or inlayed onto the substrate material. Additional materials may be added to the substrate material to alter the properties of the substrate material. For example, materials may be added to alter the surface chemistry (e.g., hydrophobicity, hydrophilicity, non-specific binding, electrostatic properties), alter the optical properties (e.g., reflective properties, refractive properties), alter the electrical or magnetic properties (e.g., dielectric materials, conducting materials, electrically-insulating materials), or alter the heat transfer characteristics of the substrate material. Additional materials contacted or adhered with a substrate material may be ordered or patterned onto the substrate material to, for example, locate the additional material at addresses or locate the additional material at interstitial regions between addresses. Exemplary additional materials may include metals (e.g., gold, silver, copper, etc.), metal oxides (e.g., titanium oxide, silicon dioxide, alumina, iron oxides, etc.), metal nitrides (e.g., silicon nitride, aluminum nitride, boron nitride, gallium nitride, etc.), metal carbides (e.g., tungsten carbide, titanium carbide, iron carbide, etc.), metal sulfides (e.g., iron sulfide, silver sulfide, etc.), and organic moieties (e.g., polyethylene glycol (PEG), dextrans, chemically-reactive functional groups, etc.).

A method of the present disclosure can include the step of coupling one or more analytes to a solid support or a surface thereof, for example, prior to performing a detection step set forth herein. The coupling of one or more analytes to a solid support surface may include covalent or non-covalent coupling of the one or more analytes to the solid support. Covalent coupling of an analyte to a solid support can include direct covalent coupling of an analyte to a solid support (e.g., formation of coordination bonds) or indirect covalent coupling between a reactive functional group of the analyte and a reactive functional group that is coupled to the solid support (e.g., a CLICK-type reaction). Non-covalent coupling can include the formation of any non-covalent interaction between an analyte and a solid support, including electrostatic or magnetic interactions, or non-covalent bonding interactions (e.g., ionic bonds, van der Waals interactions, hydrogen bonding, etc.). The skilled person will readily recognize that the particular analyte and the choice of solid support can affect the selection of a coupling chemistry for the compositions and methods set forth herein.

Accordingly, a coupling chemistry may be selected based upon the criterium that it provides a sufficiently stable coupling of an analyte to a solid support for a time scale that meets or exceeds the time scale of a method as set forth herein. For example, a polypeptide identification method can require a coupling of the analyte to the solid support for a sufficient amount of time to permit a series of empirical measurements of the analyte to occur. An analyte may be continuously coupled to a solid support for an observable length of time such as, for example, at least about 1 minute, 1 hour (hr), 3 hrs, 6 hrs, 12 hrs, 1 day, 1.5 days, 2 days, 3 days, 1 week (wk), 2 wks, 3 wks, 1 month, or more. The coupling of an analyte to a solid support can occur with a solution-phase chemistry that promotes the deposition of the analyte on the solid support. Coupling of an analyte to a solid support may occur under solution conditions that are optimized for any conceivable solution property, including solution composition, species concentrations, pH, ionic strength, solution temperature, etc. Solution composition can be varied by chemical species, such as buffer type, salts, acids, bases, and surfactants. In some configurations, species such as salts and surfactants may be selected to facilitate the formation of interactions between an analyte and a solid support. Covalent coupling methods for coupling an analyte to a solid support may include species such as catalyst, initiators, and promoters to facilitate particular reactive chemistries.

An array of analytes may be provided for a method, composition, system, or apparatus set forth in the present disclosure. Although analytes are exemplified as proteins throughout the present disclosure, it will be understood that other analytes may be provided in a similar array format. Exemplary analytes include, but are not limited to, cells, organelles, biomolecules, polysaccharides, nucleic acids, lipids, metabolites, hormones, vitamins, enzyme cofactors, therapeutic agents, candidate therapeutic agents, or combinations thereof. An analyte can be a non-biological atom or molecule, such as a synthetic polymer, metal, metal oxide, ceramic, semiconductor, mineral, or a combination thereof.

An array of analytes may be provided on a solid support containing a plurality of discrete analyte-binding sites. The analyte-binding sites may be present at addresses. Each analyte-binding site may be separated from each other analyte-binding site by one or more interstitial regions. For example, each analyte-binding site may be located at a respective address, wherein the addresses are separated from each other by one or more interstitial regions. An array interstitial region may be configured to inhibit binding of analytes or other moieties to the interstitial region, for example by containing a surface coating or layer. Exemplary interstitial region surface layers or coatings can include hydrophobic moieties (e.g., hexmethyldisilazane, alkyl moieties) or hydrophilic moieties (e.g., polyethylene glycol moieties). Surface layers or coatings provided at an interstitial region can comprise linear, branched, or dendrimeric moieties. A surface layer or coating provided at an interstitial region may be a self-assembled monolayer. An address can include a single analyte-binding site (i.e. one and only one analyte-binding site or, alternatively, a plurality of analyte-binding sites can be present at a given address.

Array analyte-binding sites can comprise one or more moieties that are coupled or otherwise bound to a solid support at the analyte-binding site. Moieties may be bound to a solid support at an analyte-binding site for facilitating coupling of an analyte to the analyte-binding site, or to inhibit unwanted binding of moieties to the analyte-binding site. Moieties may be covalently or non-covalently bound to a solid support at an analyte-binding site.

An analyte-binding site may be provided with one or more moieties that couple an analyte to the analyte-binding site. Coupling moieties can include non-covalent coupling moieties (e.g., oligonucleotides, receptor-ligand binding pairs, electrically-charged moieties, magnetic moieties, etc.), or covalent coupling moieties (e.g., Click-type reactive groups, etc.). An analyte-binding site may be provided with one or more passivating moieties that inhibit unwanted or unexpected binding of moieties to the analyte-binding site. Exemplary passivating moieties can include polymeric molecules such as polyethylene glycol (PEG), bovine serum albumin, pluronic F-127, polyvinylpyrrolidone, and Teflon, or hydrophobic materials such as hexamethyldisilazane. A passivating moiety may be covalently or non-covalently bound to a solid support at an analyte-binding site. An analyte-binding site may contain a covalently bound passivating moiety and a non-covalently bound passivating moiety. For example, an analyte-binding site may contain a PEG moiety that is covalently attached to the solid support at the analyte-binding site and a bovine serum albumin moiety that is electrostatically bound to the analyte-binding site.

An analyte-binding site may comprise a plurality of moieties coupled to a solid support. The plurality of moieties can include a coupling moiety and an optional plurality of passivating moieties. Preferably, a moiety containing a coupling moiety may further comprise a passivating moiety. For example, an oligonucleotide coupling moiety may further comprise a PEG passivating moiety. In some configurations, each individual moiety of a plurality of moieties coupled to an analyte-binding site can contain a coupling moiety. Alternatively, in some configurations, only a fraction of moieties of a plurality of moieties coupled to an analyte-binding site may contain a coupling moiety. Coupling moieties and passivating moieties may be provided at an analyte-binding site in a ratio of at least about 1000:1, 100:1, 10:1, 5:1, 2:1, 1:1, 1:2, 1:5, 1:10, 1:100, or 1:1000 coupling-to-passivating moieties. Alternatively or additionally, coupling moieties and passivating moieties may be provided at an analyte-binding site in a ratio of no more than about 1:1000, 1:100, 1:10, 1:5, 1:2, 1:1, 2:1, 5:1, 10:1, 100:1, or 1000:1 coupling-to-passivating moieties.

Analyte-binding sites may have an average characteristic dimension of at least about 10 nm, 25 nm, 50 nm, 100 nm, 150 nm, 200 nm, 250 nm, 300 nm, 500 nm, 1 □m, or more than 1 μm. Alternatively or additionally, analyte-binding sites may have an average characteristic dimension of no more than about 1 μm, 500 nm, 300 nm, 250 nm, 200 nm, 150 nm, 100 nm, 50 nm, 25 nm, 10 nm, or less than 10 nm.

Analytes may be attached directly to analyte-binding sites, for example, by coupling of a moiety attached to an analyte to a moiety attached to an analyte-binding site. Alternatively, analytes may be attached to analyte-binding sites by an anchoring moiety. An anchoring moiety may attach an analyte to an analyte-binding site, and optionally orient the analyte and/or occlude additional analytes from attaching to the analyte-binding site. An anchoring moiety may comprise a nanoparticle, such as a metal nanoparticle, a metal oxide nanoparticle, a semiconductor nanoparticle, a carbon nanoparticle, or a polymeric nanoparticle. Preferably, an anchoring moiety may comprise a nucleic acid nanoparticle. A nucleic acid nanoparticle of an anchoring moiety may comprise a first face containing one or more coupling moieties, and a second face containing an analyte-coupling site. The first face and the second face of the anchoring moiety may be substantially opposed. The anchoring moiety may further comprise a linking moiety that attaches the analyte to the anchoring moiety. The linking moiety may spatially separate the analyte from the surface of the array, for example by a distance of at least about 5 nm, 10 nm, 15 nm, 20 nm, 25 nm, 30 nm, 40 nm, 50 nm, or more than 50 nm. The linking moiety may comprise a flexible linker (e.g., a PEG or alkyl moiety) or a rigid linker (e.g., a double-stranded nucleic acid linker). An anchoring moiety may be attached to one and only one analyte. An anchoring moiety may be attached to more than one analyte. Additional aspects of anchoring moieties are described in U.S. Pat. Nos. 11,203,612, and 11,505,796, each of which is incorporated herein by reference in its entirety.

An array of analytes may be provided with a characterized or characterizable analyte-binding site occupancy. The analyte-binding site occupancy can be measured as the fraction or percentage of analyte-binding sites of a plurality of analyte-binding sites containing an attached analyte. An array of analytes may be provided with an analyte-binding site occupancy of at least about 10%, 20%, 25%, 30%, 35%, 40%, 45%, 50%, 60%, 70%, 80%, 90%, 95%, 99%, 99.9%, or more than 99.9%. Alternatively or additionally, an array of analytes may be provided with an analyte-binding site occupancy of no more than about 99.9%, 99%, 95%, 90%, 80%, 70%, 60%, 50%, 45%, 40%, 35%, 30%, 25%, 20%, 10%, or less than 10%.

An array of analytes may be provided with a fraction or percentage of individual sites that each contain one and only one analyte. The fraction or percentage may be calculated relative to all other sites in the array including, but not limited to, those containing no analyte and those containing multiple analytes. Preferably, an array of analytes may be provided with super Poisson loading of single analytes (i.e., a fraction or percentage of attachments sites containing one and only one analyte exceeding 37%). An array of analytes may be provided with at least about 10%, 20%, 25%, 30%, 35%, 37%, 40%, 45%, 50%, 60%, 70%, 80%, 90%, 95%, 99%, 99.9%, or more than 99.9% of analyte-binding sites containing one and only one analyte. Alternatively or additionally, an array of analytes may be provided with no more than about 99.9%, 99%, 95%, 90%, 80%, 70%, 60%, 50%, 45%, 40%, 35%, 30%, 25%, 20%, 10%, or less than 10% of analyte-binding sites containing one and only one analyte.

It may be especially useful to provide an array of analytes with a diversity of polypeptide species. The diversity of polypeptide species may be measured with respect to a proteome, sub-proteome (e.g., a tissue proteome, a cell proteome, an organelle proteome, a metabolome, a signalome, an albuminome, etc.), or a microbiome. An array of analytes may be provided with a diversity of polypeptide species as measured by total number of polypeptide species, percentage of species of a proteome, subproteome, or microbiome, number of proteoforms of a polypeptide species, or polypeptide dynamic range.

An array of analytes may be provided with more than one unique species of polypeptide. A first polypeptide may be considered unique from a second polypeptide if the amino acid sequences of the first polypeptide and second polypeptide differ. An array of analytes may be provided with at least about 2, 5, 10, 50, 100, 500, 1000, 2000, 5000, 10000, 15000, 20000, 25000, 30000, 40000, 500000, 100000, or more than 100000 unique species of polypeptides. Alternatively or additionally, an array of analytes may be provided with no more than about 100000, 50000, 40000, 30000, 25000, 20000, 15000, 10000, 5000, 2000, 1000, 500, 100, 50, 10, 5, 2, or less than 2 unique species of polypeptides.

An array of analytes may be provided with a fraction or percentage of species of a proteome, subproteome, or microbiome. An array of analytes may be provided with at least about 0.1%, 1%, 5%, 10%, 15%, 20%, 25%, 30%, 35%, 40%, 45%, 50%, 55%, 60%, 65%, 70%, 75%, 80%, 85%, 90%, 95%, 99%, 99.9%, or more than 99.9% of polypeptide species of a proteome, subproteome, or microbiome. Alternatively or additionally, an array of analytes may be provided with no more than about 99.9%, 99%, 95%, 90%, 85%, 80%, 75%, 70%, 65%, 60%, 55%, 50%, 45%, 40%, 35%, 30%, 25%, 20%, 15%, 10%, 5%, 1%, 0.1%, or less than 0.1% of polypeptide species of a proteome, subproteome, or microbiome.

An array of analytes may be provided with more than one proteoform of a polypeptide species. An array of analytes may be provided with more than one proteoform for two or more unique polypeptide species. Types of proteoforms of a polypeptide species can include coding variation proteoforms, translational variation proteoforms, post-translational modification proteoforms, splice variants, and combinations thereof. An array of analytes may be provided with at least about 2, 3, 4, 5, 10, 20, 50, 100, 200, 500, 1000, or more than 1000 proteoforms of a polypeptide species. Alternatively or additionally, an array of analytes may be provided with no more than about 1000, 500, 200, 100, 50, 20, 10, 5, 4, 3, or less than 3 proteoforms of a polypeptide species.

An array of analytes may be provided with a dynamic range of polypeptides. Dynamic range can refer to the ratio of abundance between a more populous polypeptide species and a less populous polypeptide species. A dynamic range can be an absolute measure (ratio of most populous polypeptide species to least populous polypeptide species) or a relative measure (ratio of a first particular polypeptide species to a second particular polypeptide species). An array of analytes may be provided with a dynamic range of at least about 10, 10², 10³, 10⁴, 10⁵, 10⁶, 10⁷, 10⁸, 10⁹, 10¹⁰, 10¹¹, 10¹², or more than 10¹². Alternatively or additionally, an array of analytes may be provided with a dynamic range of no more than about 10¹², 10¹¹, 10¹⁰, 10⁹, 10⁸, 10⁷, 10⁶, 10⁵, 10⁴, 10², 10², 10, or less than 10.

In some methods, providing an array of analytes may further comprise forming the array of analytes. An array of analytes may be formed by a process that includes a step of coupling analytes to analyte-binding sites of the array. An analyte may be coupled to an analyte-binding site by coupling of a coupling moiety attached to the analyte to a compatible coupling moiety attached to the analyte-binding site. In some cases where an analyte is attached to an anchoring moiety, a step of coupling the analyte to the analyte-binding site may comprise coupling the anchoring moiety to the analyte-binding site. In particular cases, an analyte may be coupled to an analyte-binding site by coupling of a coupling moiety attached to an anchoring moiety to a compatible coupling moiety attached to the analyte-binding site.

When forming an array of analytes, a plurality of analytes may be provided in a fluidic medium. A fluidic medium containing a plurality of analytes may be contacted to a solid support comprising a plurality of analyte-binding sites. After contacting the fluidic medium comprising the analytes to the solid support, analytes may couple to analyte-binding sites, thereby forming the array of analytes. In some cases, after contacting a fluidic medium containing analytes to a solid support containing analyte-binding sites, a mass transfer process may occur to facilitate coupling of the analytes to the analyte-binding sites. A mass transfer process can include chemical or mechanical processes that increase a rate of mass transfer of analytes to the surface of the solid support containing the analyte-binding sites. Chemical methods can include altering a pH (e.g., increasing the pH, decreasing the pH), ionic strength (e.g., increasing the ionic strength, decreasing the ionic strength), or temperature (e.g., increasing the temperature, decreasing the temperature) of a fluidic medium containing analytes. A chemical method of increasing mass transfer of analytes may depend upon the chemical composition of the analytes or moieties attached thereto (e.g., anchoring moieties). For example, an analyte attached to a nucleic acid nanoparticle (or any other particle having a net negative electrical surface charge) may transfer toward a hydrophobic surface more readily if the ionic strength of the fluidic medium is decreased. Mechanical methods of increasing mass transfer can include any suitable method of imparting a force on an analyte or a moiety attached thereto, such as centrifugation, electrophoresis, or magnetic attraction. Accordingly, it may be useful to provide an analyte attached to an electrically-charged particle, a magnetic particle, a particle that is denser than a fluidic medium, or a combination thereof.

A method of forming an array of analytes may include repeating one or more steps of attaching analytes to analyte-binding sites of the array. It may be preferable to repeat certain analyte-coupling steps to increase the analyte-binding site occupancy of an array of analytes. Fluidic media containing analytes may be repetitively or sequentially contacted to a solid support. A method of forming an array of analytes may further include a rinsing step (e.g., after contacting a fluidic medium to a solid support), thereby removing unbound or weakly-bound analytes or other moieties (e.g., anchoring moieties) from contact with the solid support.

It may be useful to provide an array of analytes with one or more fiducial elements. A fiducial element may comprise a detectable address or region of the array that facilitates spatial identification on the array. A fiducial element may provide a landmark or fixed reference for determining position on an array, a measurement of length or distance on the array, a spatial reference for calibrating a detection device (e.g., a sensor or camera), and a spatial reference for registering the addresses of analyte-binding sites consistently over the timespan of an array-based process. In some cases, fiducial elements may be disposed in interstitial regions of a solid support. In other cases, fiducial elements may be disposed at analyte-binding sites of the solid support.

A fiducial element may be formed on a surface of a solid support, preferably at an interstitial region. A fiducial element can be formed by etching the surface of a solid support, thereby providing a fiducial element as a protrusion or depression of the solid support material. Alternatively, a fiducial element can be formed by depositing and/or forming a material (e.g., a metal, metal oxide, or semiconductor) on the surface of the solid support. Alternatively, a particle can be deposited on a surface of the solid support. Various lithographic techniques may be useful for providing fiducial elements on solid support surfaces at fixed addresses and with useful shapes or morphologies. A plurality of fiducial elements can be formed on a solid support at regular or patterned addresses or regions, thereby providing spatial landmarks or a scale for determining relative or absolute distance. Alternatively, fiducial elements may be formed at random locations.

Fiducial elements may be deposited at analyte-binding sites of an array of analytes. It may be preferable to deposit fiducial elements at analyte-binding sites with a random spatial distribution (e.g., providing fiducial elements at a set of analyte-binding sites having no identifiable short-range or long-range spatial pattern). A random spatial distribution of fiducial elements at analyte-binding sites may provide a spatial reference that facilitates analyte-binding site registration when detectable signals are produced from a relatively small quantity of analyte-binding sites (e.g., detectable signals produced from less than about 25%, 20%, 15%, 10%, 5%, 1%, 0.5%, or 0.1% of analyte-binding sites). It may be preferable to provide fiducial elements at at least about 0.01%, 0.05%, 0.1%, 0.5%, 1%, or more than 1% of analyte-binding sites. Alternatively or additionally, fiducial elements may be provided at no more than about 1%, 0.5%, 0.1%, 0.05%, 0.01%, or less than 0.01% of analyte-binding sites. In a preferable configuration, an analyte-binding site may contain one and only one fiducial element. In other configurations, an analyte-binding site may contain more than one fiducial element.

Fiducial elements attached to analyte-binding sites of an array of analytes may comprise a material that provides a detectable signal (e.g., a fluorescent or luminescent signal). Exemplary materials for fiducial elements can include quantum dots or fluorescently-labeled polymer particles. In some cases, a fiducial element may further comprise a coupling moiety that couples to a complementary coupling moiety that is attached to an analyte-binding site. For example, a fiducial element may be functionalized with one or more oligonucleotides that comprise a nucleotide sequence that is complementary to a nucleotide sequence of a site-coupled oligonucleotide. In some cases, a fiducial element and an analyte may be coupled to individual analyte-binding sites by the same type of coupling moiety. For example, fiducial elements and analytes may be attached to individual analyte-binding sites by hybridization of oligonucleotides on the fiducial elements or analytes to complementary oligonucleotides at analyte-binding sites. Alternatively, a fiducial element and an analyte may be coupled to individual analyte-binding sites by a different type of coupling moiety. For example, a fiducial element may be attached electrostatically to a first oligonucleotide-containing analyte-binding site while an analyte may be attached by nucleic acid hybridization to a second oligonucleotide-containing analyte-binding site.

Fiducial elements may be provided at analyte-binding sites to facilitate multiplexed detection of analytes. In configurations utilizing more than one wavelength of light for optical detection, it may be preferable to provide fiducial elements that provide corresponding signals for each detected wavelength of light. For example, in a four-color detection system, it may be preferable to provide fiducial elements that provide signals for each of the four colors of light detected by the system. In some configurations, a fiducial element may be provided on an array that produces signals for each detected wavelength of light (e.g., a multi-color fluorescently-labeled polymer particle).

Fiducial elements may be deposited on an array before or after analytes are deposited on the array. A fluidic medium containing a plurality of fiducial elements may be contacted to a solid support containing a plurality of analyte-binding sites. After contacting the fluidic medium to the solid support, fiducial elements may couple to individual analyte-binding sites, preferably in a random spatial distribution. Alternatively, fiducial elements and analytes may be deposited simultaneously. A fluidic medium comprising a plurality of analyte and a plurality of fiducial elements may be contacted to a solid support containing a plurality of analyte-binding sites. After contacting the fluidic medium to the solid support, fiducial elements and analytes may couple to individual analyte-binding sites, preferably in a random spatial distribution. Additional aspects of arrays containing fiducial elements are described in U.S. Patent Publication No. 2023/0314324 A1, which is herein incorporated by reference in its entirety.

An analyte or affinity reagent can be attached to a retaining component such as a particle, array address, solid support or other substance. A particularly useful retaining component is a structured nucleic acid particle (SNAP). SNAPS can optionally include nucleic acid origami. A nucleic acid origami can include one or more nucleic acids folded into a variety of overall shapes such as a disk, tile, cylinder, cone, sphere, cuboid, tubule, pyramid, polyhedron, or combination thereof. Examples of structures formed with DNA origami are set forth in Zhao et al. Nano Lett. 11, 2997-3002 (2011); Rothemund Nature 440:297-302 (2006); Sigle et al, Nature Materials 20:1281-1289 (2021); or U.S. Pat. No. 8,501,923 or U.S. Pat. No. 9,340,416, each of which is incorporated herein by reference. In some configurations, a structured nucleic acid particle can include a nucleic acid nanoball and the nucleic acid nanoball can include a concatemeric repeat of amplified nucleotide sequences. The concatemeric amplicons can include complements of a circular template amplified by rolling circle amplification. Exemplary nucleic acid nanoballs and methods for their manufacture are described, for example, in U.S. Pat. No. 8,445,194, which is incorporated herein by reference. Further examples of structured nucleic acid particles are set forth in US Pat. Nos. 11,203,612 or 11,505,796; or US Pat. App. Pub. No. 2022/0162684 A1 or 2023/0167488 A1, each of which is incorporated herein by reference.

A retaining component, such as a SNAP, may have any of a variety of sizes and shapes to accommodate use in a desired application. For example, a retaining component can have a regular or symmetric shape or, alternatively, it can have an irregular or asymmetric shape. The shape can be rigid or pliable. The size or shape of a SNAP or other retaining component can be characterized with respect to length, area (i.e. footprint), or volume. The size or shape of a SNAP or other retaining component can be smaller than an address in an array to which it will associate or attach. Optionally, the relative sizes and shapes of an individual retaining component and an address to which it will attach are configured to preclude more than one of the retaining components from occupying the address.

Optionally, a retaining component (e.g. SNAP) or population thereof has a minimum, maximum or average length of at least about 50 nm, 100 nm, 250 nm, 500 nm, 1 micron, 5 micron or more. Alternatively or additionally, a retaining component (e.g. SNAP) or population thereof has a minimum, maximum or average length of no more than about 5 micron, 1 micron, 500 nm, 250 nm, 100 nm, 50 nm, or less.

Optionally, a retaining component (e.g. SNAP) or population thereof has a minimum, maximum or average volume of at least about 1 micron³, 10 micron³, 100 micron³, 1 mm³ or more. Alternatively or additionally, a retaining component (e.g. SNAP) or population thereof has a minimum, maximum or average volume of no more than about 1 mm³, 100 micron³, 10 micron³, 1 micron³ or less.

Optionally, the minimum, maximum or average area (i.e. footprint) for a retaining component (e.g. SNAP) is at least about 10 nm², 100 nm², 1 micron², 10 micron², 100 micron², 1 mm² or more. Alternatively or additionally, the minimum, maximum or average area for a retaining component (e.g. SNAP) footprint is at most about 1 mm², 100 micron², 10 micron², 1 micron², 100 nm², 10 nm², or less. The footprint of a retaining component (e.g. SNAP) may have a regular shape or an approximately regular shape, such as triangular, square, rectangular, circular, ovoid, or polygonal shape.

A structured nucleic acid particle (e.g. having origami or nanoball structures) may include regions of single-stranded nucleic acid, regions of double-stranded nucleic acid, or combinations thereof. For example, a SNAP can have a nucleic acid origami structure which includes a scaffold strand and a plurality of staple strands. The scaffold strand can be configured as a single, continuous strand of nucleic acid, and the staples can be formed by nucleic acid strands that hybridize, in whole or in part, with the scaffold strand.

In some configurations, a nucleic acid origami includes a scaffold composed of a nucleic acid strand to which a plurality of oligonucleotides is hybridized. A nucleic acid origami may have a single scaffold molecule or multiple scaffold molecules. A scaffold strand can be linear (i.e. having a 3′ end and 5′ end) or circular (i.e. closed such that the scaffold lacks a 3′ end and 5′ end). A scaffold strand can be derived from a natural source, such as a viral genome or a bacterial plasmid. For example, a nucleic acid scaffold can include a single strand of an M13 viral genome. In other configurations, a scaffold strand may be synthetic, for example, having a non-naturally occurring nucleotide sequence in full or in part. A scaffold nucleic acid can be single stranded but for a plurality of oligonucleotides hybridized thereto or short regions of internal complementarity. The size of a scaffold strand may vary to accommodate different uses. For example, a scaffold strand may include at least about 100, 500, 1000, 2500, 5000 or more nucleotides. Alternatively or additionally, a scaffold strand may include at most about 5000, 2500, 1000, 500, 100 or fewer nucleotides.

A nucleic acid origami can include one or more oligonucleotides that are hybridized to a scaffold strand. An oligonucleotide can include two sequence regions that are hybridized to a scaffold strand, for example, to function as a ‘staple’ that restrains the structure of the scaffold. For example, a single oligonucleotide can hybridize to two regions of a scaffold strand that are separated from each other in the primary sequence of the scaffold strand. As such, the oligonucleotide can function to retain those two regions of the scaffold strand in proximity to each other or to otherwise constrain the scaffold strand to a desired conformation. Two sequence regions of an oligonucleotide staple that bind to a scaffold strand can be adjacent to each other in the nucleotide sequence of the oligonucleotide or separated by a spacer region that does not hybridize to the scaffold strand.

An oligonucleotide can include a first sequence region that is hybridized to a complementary sequence of a nucleic acid origami and a second region that provides a “handle” or “linker” for attaching another moiety. For example, the moiety can include an analyte (e.g. protein), paratope, affinity moiety (e.g. antibody), organic linker, inorganic ion, docker or tether. Optionally, the moiety can be attached to an oligonucleotide that is complementary to the second region of the handle and the moiety can be attached to the nucleic acid origami via hybridization of the handle to the complementary oligonucleotide.

Oligonucleotides can be configured to hybridize with a nucleic acid scaffold, another oligonucleotide, a staple oligonucleotide, or a combination thereof. One or more regions of an oligonucleotide that hybridizes to another sequence of a nucleic acid origami or other structured nucleic acid particle can be located at or near 5′ end of the oligonucleotide, at or near 3′ end of the oligonucleotide, or in a region of the oligonucleotide that is between the end regions. The oligonucleotides can be linear (i.e. having a 3′ end and a 5′ end) or closed (i.e. circular, lacking both 3′ and 5′ ends). An oligonucleotide that is included in a nucleic acid origami or other structured nucleic acid particle can have any of a variety of lengths including, for example, at least about 10, 25, 50, 100, 250, 500, or more nucleotides. Alternatively or additionally, an oligonucleotide may have a length of no more than about 500, 250, 100, 50, 25, 10, or fewer nucleotides. An oligonucleotide may form a hybrid of at least about 5, 6, 7, 8, 9, 10, 15, 20, 25, 50 or more consecutive or total base pairs with another nucleotide sequence of a nucleic acid origami. Alternatively or additionally, an oligonucleotide may form a hybrid of no more than about 50, 25, 20, 15, 10, 9, 8, 7, 6, 5, or fewer consecutive or total base pairs with another nucleotide sequence.

A retaining component may be provided with moieties that facilitate a binding interaction with a surface of a solid support, or moieties coupled to the surface of the solid support. Moieties that facilitate coupling of a retaining component to a solid support may be configured to form a covalent interaction or a non-covalent interaction with the solid support or a moiety coupled to the solid support. In an example, a retaining component may be provided with one or more nucleic acid strands that can hybridize to a complementary nucleic acid strand on a surface of a solid support by nucleic acid hybridization. Preferably, a retaining component may be provided with a plurality of moieties that can bind to a surface of a solid support. In some cases, the moieties may be pendant from the retaining component. Pendant moieties may include a linking moiety that increases the length of the moiety and/or increases the flexibility or spatial degrees of freedom of the moiety. A linking moiety can be, for example, a single-stranded nucleic acid (e.g., with a nucleotide sequence that is not complementary to a surface-bound oligonucleotide), a peptide linker, or a synthetic polymer (e.g., polyethylene glycol, alkyl moieties, etc.).

A structured nucleic acid particle (e.g., nucleic acid origami, or nucleic acid nanoball) may be formed by an appropriate technique including, for example, those known in the art. Nucleic acid origami can be designed, for example, as described in Rothemund, Nature 440:297-302 (2006), or U.S. Pat. No. 8,501,923 or U.S. Pat. No. 9,340,416, each of which is incorporated herein by reference. Nucleic acid origami may be designed using a software package, such as CADNANO (cadnano.org), ATHENA (github.com/lcbb/athena), or DAEDALUS (daedalus-dna-origami.org).

Other useful retaining components include artificial polymers. Artificial polymers can include polymers that are made by human activity rather than occurring naturally. For example, a polymer that is made at least in part by human activity or that includes at least one artificial moiety is referred to as an “artificial polymer.” In some cases the artificial polymers are configured as dendrons. A dendron will include at least one branched chain polymer. A branched chain polymer can include at least 1, 2, 3, 4, 5, 6, 8 or 10 branch points. Alternatively or additionally, a branched chain can include at most 10, 8, 6, 5, 4, 3, 2 or 1 branch points. A branch point is a covalent intersection between at least two chains. For example, at least 2, 3, 4, 5 or more chains can intersect at a branch point of a branched chain. Alternatively or additionally, at most 5, 4, 3 or 2 chains can intersect at a branch point of a branched chain. A polymer, whether branched or not, can include a single type of monomer subunit or multiple different types of monomer subunits. Accordingly, a polymer can include at least 1, 2, 3, 4, 5 or more different types of monomer subunits. Alternatively or additionally, a polymer can include at most 5, 4, 3, 2 or 1 different types of monomer subunits. A polymer having only one type of subunit in the network of covalent bonds is referred to as a “homopolymer.” In contrast, a “copolymer” includes two or more different types of subunits in the network of covalent bonds.

A retaining component that includes an artificial polymer can have a length, volume or footprint in a range set forth above. A retaining component can be further characterized in terms of molecular weight (or molecular weight distribution) in a desired size range. For example, the molecular weight, average molecular weight distribution, minimum molecular weight distribution or maximum molecular weight distribution can be at least 1 kDa, 2 kDa, 5 kDa, 10 kDa, 25 kDa, 50 kDa or more. Alternatively or additionally, the molecular weight, average molecular weight distribution, minimum molecular weight distribution or maximum molecular weight distribution can be at most 50 kDa, 25 kDa, 10 kDa, 5 kDa, 2 kDa, 1 kDa or less. A retaining component can be characterized in terms of radius of gyration. For example, the radius of gyration can be at least about 2 nm, 5 nm, 10 nm, 15 nm, 25 nm, 50 nm or more. Alternatively or additionally, retaining component can be configured to have a radius of gyration that is at most about 50 nm, 25 nm, 15 nm, 10 nm, 5 nm, 2 nm or less. An artificial polymer can be characterized in term of degree of polymerization (i.e. number of monomer subunits) present. For example, an artificial polymer can include at least 2, 10, 20, 30, 40, 50, 100, 200, 300 or more monomers. Alternatively or additionally, an artificial polymer can include at most 300, 200, 100, 50, 40, 30, 20, 10, or 2 monomers.

An artificial polymer can lack natural polymers or monomers found in natural polymers. For example, the skeletal structure of the artificial polymer can lack natural polymers or monomers. This can be the case whether or not the artificial polymer has attached moieties that include natural polymers or monomers. Examples of natural moieties that can be absent from an artificial polymer, for example in the skeletal structure include, but are not limited to, nucleic acids (e.g. DNA or RNA), nucleotides (e.g. deoxyribonucleotides or ribonucleotides), nucleosides (e.g. deoxyribonucleosides or ribonucleosides), peptides (e.g. proteins, polypeptides or oligopeptides), amino acids, or sugars (e.g. saccharide monomers, monosaccharides, oligosaccharides, polysaccharides or glycans). An artificial polymer can optionally lack any polymer or monomer that is synthesized in vivo or that is capable of being synthesized in vivo. Alternatively, an artificial polymer can include natural moieties that are combined to form a non-naturally occurring molecule. For example, an artificial polymer can be composed of nucleic acid monomers or nucleic acid strands that form a non-naturally occurring nucleic acid dendrimer structure.

Particularly useful artificial polymers include, for example, poly(amidoamine) (PAMAM) dendrimer, poly(amidoamine) dendron, hyperbranched polymers such as linear and branched polyethyleneimine (PEI) and polypropyleneimine (PPI), star polymers, grafted polymers, peptide-based linear or branched dendrimers such as branched poly-L-lysine (PLL) and silane-cored dendrimer. Other useful artificial polymers include dendrimer nucleic acids having branching structures. See, for example, Liu et al., J. Mater. Chem. B 9:4991-5007 (2021) and Meng et al., ACS Nano 8:6171-6181 (2014), each of which is incorporated herein by reference. Examples of useful polymers are set forth in Tomalia, et al. J Polym Sci Part A: Polym Chem 40:2719-2728 (2002); Higashihara, et al. Polym J 44, 14-29 (2012); Gupta, et al. J. Phys. Chem. B 124, 20, 4193-4202 (2020); Ren, et al. Chem. Rev. 116, 12, 6743-6836 (2016); Chis, et al. Molecules 25 (17): 3982 (2020); Zheng, et al. or Chem. Soc. Rev. 44, 4091-4130 (2015), each of which is incorporated herein by reference.

The present disclosure provides compositions and methods for improving binding of analytes to affinity reagents by increasing avidity of the binding interaction. In particular embodiments, avidity between an analyte and affinity reagent can be increased by association of a docker with the analyte and association of a tether with the affinity reagent. The docker and tether recognize each other and can thus bind to each other. Avidity of the interaction between the affinity reagent and analyte is a function not only of recognition between the paratope and epitope, but also recognition between the docker and tether.

A docker can be associated with an analyte via covalent and/or non-covalent attachment of the docker to the analyte. Similarly, a tether can be associated with an affinity reagent via covalent and/or non-covalent attachment of the docker to the affinity reagent. Exemplary attachment chemistries include those set forth herein in the context of attaching analytes and affinity reagents to retaining components, addresses of an array, solid supports, labels, etc. In some configurations, a docker or tether can be attached to a particle (e.g. structured nucleic acid particle), unique identifier, address or solid support to which an analyte or affinity reagent, respectively, is attached.

Accordingly, the present disclosure provides a method of processing an analyte. The method can include the steps of (a) providing an analyte comprising an epitope and a docker; (b) providing an affinity reagent, wherein the affinity reagent comprises a paratope that recognizes the epitope and a tether that recognizes the docker; and (c) contacting the analyte with the affinity reagent, whereby the affinity reagent associates with the analyte via binding of the paratope to the epitope and via binding of the tether to the docker. Optionally, the method further includes a step of detecting association of the affinity reagent with the analyte, thereby identifying the analyte. In another option, the analyte is present in a sample including other analytes and the method further includes a step of separating the analyte from the other analytes via the association of the affinity reagent with the analyte.

The compositions and methods of the present disclosure are particularly well suited for detecting analytes using affinity reagents in non-equilibrium conditions. A typical binding assay employ an excess amount of affinity reagent and immobilized analytes to drive formation of an immobilized complex between the affinity reagent and analyte. In some assays the excess labeled affinity reagent in solution produces unwanted background that overwhelms signal produced by immobilized complexes. Removal of excess affinity reagents from solution creates a non-equilibrium condition that drives affinity reagents to dissociate from the immobilized analytes. The use of tethers and dockers can increase the half-life of the complexes under non-equilibrium conditions, thereby improving detectability of analyte-affinity reagent complexes.

A variety of different types of dockers and tethers can be employed to increase avidity of binding between an analyte and affinity reagent. The type of docker and tether that is to be used in combination with a particular analyte and affinity reagent pair can be selected based on known or expected affinity of the affinity reagent for the analyte. For example, a method that employs a first affinity reagent having relatively strong affinity for a particular analyte can utilize a docker and tether pair having relatively weak affinity, whereas a method that employs a second affinity reagent having weaker affinity for the analyte can utilize a docker and tether pair having higher affinity compared to the pair used for the first affinity reagent. Accordingly, the probability of forming a complex and duration of the complex can be tuned by appropriate choice of docker type and tether type.

A docker can be any molecule or moiety that is capable of binding to a tether and a tether can be any molecule or moiety that is capable of binding to a docker. A particularly useful docker or tether is a nucleic acid strand having a nucleotide sequence that complements a nucleotide sequences of a tether or docker, respectively. A nucleic acid strand that is used as a docker or tether can include a sequence of at least 3, 4, 5, 6, 7, 8, 9, 10, 12, 14, 16, 18, 20, 25 or more nucleotides. Alternatively or additionally, a nucleic acid strand that is used as a docker or tether can include a sequence of at most 25, 20, 18, 16, 14, 12, 10, 9, 8, 7, 6, 5, 4, 3 or fewer nucleotides. Other useful dockers or tethers include, for example, a receptor that recognizes a ligand, a ligand that recognizes a receptor, an affinity reagent that recognizes an analyte, an analyte that recognizes an affinity reagent, a paratope that recognizes an epitope, an epitope that recognizes a paratope, or a reactive moiety that forms a covalent bond with another reactive moiety. Exemplary dockers or tethers include, but are not limited to, an antibody, Fab′ fragment, F(ab′)2 fragment, single-chain variable fragments, di-scFv, tri-scFv, microantibody, nucleic acid aptamer, affibody, affilin, affimer, affitin, alphabody, anticalin, avimer, miniprotein, DARPin, monobody, nanoCLAMP, lectin, carbohydrate, SpyCatcher or SpyTag. In some configurations, a docker or tether can be a protein that recognizes a nucleic acid sequence such as a DNA binding protein or RNA binding protein. Exemplary nucleic acid-binding proteins, which can be used as dockers or tethers, and the nucleic acid moieties to which they bind, which can be used as tethers or dockers, respectively, include a Toll-Like Receptor (TLR) which binds to DNA having a CpG moiety, transcription factor which binds to a specific nucleic acid sequence, or histone protein(s) which binds to DNA. Further examples are provided in the Eukaryotic nucleic acid binding protein database (ENPD). See Leung et al. Nucleic Acids Res. 47 (Database issue): D322-D329 (2019), which is incorporated herein by reference.

A further variable that can be employed to tune binding between an analyte and affinity reagent is the number of dockers associated with the analyte and/or the number of tethers associated with the affinity reagent. For example, a method that employs a first affinity reagent having relatively strong affinity for an analyte can utilize a relatively low number of docker-tether pairs, whereas a method that employs a second affinity reagent having weaker affinity for the analyte can utilize a greater number of docker-tether pairs compared to the number(s) used for the first affinity reagent.

An analyte can be associated with a single docker or, alternatively, with a plurality of dockers. For example, an analyte can be associated with at least 2, 3, 4, 5, 6, 7, 8, 9, 10, 15, 20, 25, 50 or more dockers. Alternatively or additionally, an analyte can be associated with at most 50, 25, 20, 15, 10, 9, 8, 7, 6, 5, 4, 3, 2 or fewer dockers. The dockers can be substantially identical to each other, thereby recognizing the same tethers. Alternatively, a plurality of dockers can include dockers that differ from each other. In some cases, the different dockers will recognize different tethers. It is also possible for the different dockers to recognize the same tethers. In some configurations, an analyte and the docker with which it is associated will have binding characteristics that are orthogonal to each other. As such, a paratope of an affinity reagent that recognizes or binds to the analyte will not recognize or bind to the docker, and a tether that recognizes or binds to the docker will not recognize or bind to the analyte.

An affinity reagent can be associated with a plurality of tethers. For example, an affinity reagent can be associated with at least 2, 3, 4, 5, 6, 7, 8, 9, 10, 15, 20, 25, 50 or more tethers. Alternatively or additionally, an affinity reagent can be associated with at most 50, 25, 20, 15, 10, 9, 8, 7, 6, 5, 4, 3, 2 or fewer tethers. The tethers can be substantially identical to each other, thereby recognizing the same dockers. Alternatively, a plurality of tethers can include tethers that differ from each other. In some cases, the different tethers will recognize different dockers. It is also possible for the different tethers to recognize the same dockers. In some configurations, an affinity reagent and the tether with which it is associated will have orthogonal binding recognition. As such, an analyte that recognizes or binds to a paratope of the affinity reagent will not recognize or bind to the tether, and a docker that recognizes or binds to the tether will not recognize or bind to the paratope.

Of course, a binding event can be tuned via a combination of the number and type of docker-tether pairs used. This can be illustrated in the context of nucleic acid dockers and tethers having complementary nucleotide sequences. For example, the maintenance of a complex between an analyte and affinity reagent can be increased by increasing the number of dockers and tethers present in the complex and also by increasing the avidity of each docker for its complementary tether. The avidity of binding between a nucleic acid docker and tether can be increased, for example, by increasing the length of the complementary sequences, increasing the GC content of the complementary sequences, or otherwise increasing the melting temperature (Tm) of the duplex formed by the complementary sequences. The length of the complementary sequences can be at least 3, 4, 5, 6, 7, 8, 9, 10, 12, 14, 16, 18, 20, 25 or more nucleotides. Alternatively or additionally, the length of the complementary sequences can be at most 25, 20, 18, 16, 14, 12, 10, 9, 8, 7, 6, 5, 4, 3 or fewer nucleotides. The GC content of the complementary sequences can be at least 25%, 40%, 50%, 60%, 75%, or higher. Alternatively or additionally, the GC content of the complementary sequences can be at most 75%, 60%, 50%, 40%, 25% or lower.

Multiplex methods, in which a plurality of different analytes are processed in parallel, can employ universal dockers. The dockers are referred to as ‘universal’ because they are identical with respect to structural features that interact with tethers. For example, an array can include a plurality of addresses, each of the addresses being attached to an analyte that differs from other analytes in the array and each of the addresses being attached to a docker that is the same as other dockers in the array. A plurality of different analytes that are associated with universal dockers can be contacted with a plurality of different affinity reagents that are associated with tethers. Some or all the different affinity reagents can have the same tether structure. As such, the avidity effect of the dockers and tethers can be substantially uniform.

Methods that employ multiple different affinity reagents can employ universal tethers. The tethers are referred to as ‘universal’ because they are identical with respect to structural features that interact with dockers. For example, an array of analytes can be contacted with a plurality of different affinity reagents, each of the affinity reagents having a paratope that differs from other affinity reagents in the plurality and each of the affinity reagents being attached to a tether that is the same as other tethers in the plurality. The different affinity reagents can be present in a mixture that is simultaneously in contact with the array or, alternatively, the different affinity reagents can be serially contacted with the array.

Compositions set forth herein can interact with each other via covalent bonds. Molecules, moieties thereof or atoms thereof can form covalent bonds with other molecules, moieties or atoms. Covalent interactions can be reversible or irreversible in the context of a method set forth herein. A covalent bond can arise due to a chemical reaction between a first reactive moiety and a second reactive moiety, optionally in the presence of a third intermediary or catalytic moiety. Covalent bonds can be formed via various chemical mechanisms, including addition, substitution, elimination, oxidation, and reduction. In some cases, a covalent binding interaction may be formed by a Click-type reaction, as set forth herein (e.g., methyltetrazine (mTz)-tetracyclooctylene (TCO), azide-dibenzocyclooctene (DBCO), thiol-epoxy). In some cases, a ligand-receptor-type binding interaction can form a covalent binding interaction. For example, SpyCatcher-Spy Tag, SnoopCatcher-SnoopTag, and SdyCatcher-Sdy Tag are receptor-ligand binding pairs that can form covalent binding interactions due to isopeptide bond formation. Additional useful covalent binding interactions can include coordination bond formation, such as between a metal-containing substrate and a ligand. Exemplary coordination bonds can include silicon-silane, metal oxide-phosphate, and metal oxide-phosphonate. Useful reagents and mechanisms for forming covalent binding interactions, including bioorthogonal binding interactions, as set forth herein, are provided in U.S. Pat. Nos. 11,203,612 or 11,505,796, each of which is herein incorporated by reference in its entirety.

Compositions set forth herein can interact with each other via non-covalent bonds. A non-covalent bond can include an electrostatic or magnetic interaction between a first moiety and a second moiety. A non-covalent bond can include electrostatic interactions such as ionic bonding, hydrogen bonding, halogen bonding, Van der Waals interactions, Pi-Pi stacking, Pi-ion interactions, Pi-polar interactions, or magnetic interactions. In some cases, a non-covalent bond may be formed by hybridization of a first oligonucleotide to a complementary second oligonucleotide. Such bonding is also known as Watson-Crick base-pairing. In some cases, a non-covalent interaction may be formed by a receptor-ligand binding pair, such as streptavidin-biotin. Other useful non-covalent interactions can include affinity reagent-target interactions, such as antibody-epitope or aptamer-epitope interactions.

Systems and methods for forming and utilizing arrays, such as those set forth herein, may contain multiple types of covalent and/or non-covalent interactions. For example, a useful array site configuration may comprise an analyte (e.g., a polypeptide) that is covalently bonded to an oligonucleotide, in which the oligonucleotide is hybridized to a nucleic acid nanoparticle, in which the nucleic acid nanoparticle is hybridized to a surface-coupled oligonucleotide, and in which the surface-coupled oligonucleotide is covalently bonded to a surface of a solid support. This example may be extended to further include an affinity reagent that is non-covalently bound to the analyte. The affinity reagent bound to the analyte, in turn, may be covalently bonded to a nanoparticle or a moiety thereof (e.g., an oligonucleotide). The skilled person will recognize that the various covalent and non-covalent interactions occurring in the system and methods set forth herein may vary with respect to both time-scale and reversibility (or lack thereof) for association and/or dissociation of the binding interactions. Accordingly, it will be recognized that certain binding interactions (e.g., covalent binding of an analyte to an oligonucleotide) will be selected to inhibit or minimize a likelihood of association or dissociation over the duration of a method, or a step thereof, as set forth herein, and other binding interactions (e.g., non-covalent binding of an affinity reagent to an analyte) will be selected to facilitate or increase a likelihood of association or dissociation within the duration of a method or a step thereof, as set forth herein.

Entities, such as affinity reagents and their binding targets, can be associated with each other and dissociated form each other in a method set forth herein. Association of a first entity to a second entity can involve a contacting step, in which the first entity is brought into proximity of the second entity, and an association step in which a first coupling moiety of the first entity forms a binding interaction with a second coupling moiety of the second entity. Dissociation of a first entity and a second entity need not be construed as a reversal of an association process between the first entity and the second entity. For example, a first entity comprising a first oligonucleotide coupled to a second entity comprising a second oligonucleotide by hybridization of the first oligonucleotide to the second oligonucleotide could be dissociated by dehybridization of the nucleic acids (thereby returning the first entity and the second entity as originally provided before association), or dissociated by enzymatic cleavage of the hybridized nucleic acids (thereby providing the first and the second entities with each individually further comprising an at least partially double-stranded cleavage product).

Systems and methods utilizing arrays, as set forth herein, may have binding interactions that are unwanted, unexpected, or contrary-to-design. The binding interactions (e.g. covalent or non-covalent) can occur between an array surface or array feature and an unbound moiety that may become contacted with the array surface or array feature. Such interactions may be referred to hereinafter as “orthogonal binding phenomena.” Orthogonal binding phenomena may be qualitatively characterized as a binding interaction that occurs in a system that is expected or engineered to prevent such a binding interaction (e.g., a putatively hydrophilic molecule binding to a putatively hydrophobic surface). Orthogonal binding phenomena may be quantitatively characterized as measurable binding interactions occurring between an array surface or array feature (e.g., an interstitial region, an analyte address) and moiety that may transition from a fluid-phase state to an immobilized state, for example, by attaching to the array surface or feature, wherein the measurable binding interactions occur at a rate and/or to an extent that exceeds a predicted rate and/or extent. For example, the rate or extent may exceed a thermodynamic or kinetic prediction (e.g., a dissociation constant, a binding on-rate, a binding off-rate, etc.). By way of further example, if an unbound moiety is characterized to bind to a surface-coupled passivating moiety (e.g., polyethylene glycol) with a kilomolar dissociation constant (a very weak binding interaction), then observing a millimolar binding dissociation constant between the unbound moiety and an array surface that is provided with a uniform layer of the surface-coupled passivating moiety would indicate an orthogonal binding phenomena (i.e., binding due to a mechanism other than the specific binding of the unbound moiety to the surface-coupling passivating moiety). Orthogonal binding phenomena may be characterized based upon a stochastic measure, such as spatial and/or temporal variations in unwanted, unexpected, or contrary-to-design binding phenomena.

The presence, type or degree of orthogonal binding phenomenon is typically contextual, for example, relating to the conditions in which a binding interaction occurs. Consider, for example, a surface that is engineered to comprise a material that prevents binding of an assay reagent to the surface but also contains an unwanted impurity or a vacancy that can form a binding interaction with the assay reagent. Independent of any predictability or specificity that may be ascribed to the binding interaction between the impurity and the assay reagent, it is unwanted and unintended. Accordingly, “non-orthogonal” and “orthogonal” binding interactions can be discerned in the context of the intended use of the system within which they occur. In the context of a molecular array, such as a single-analyte array, the molecular array may be engineered to specifically retain chosen moieties at particular sites and inhibit binding of particular moieties at other sites or regions. In such a context, orthogonal binding interactions may include binding of moieties other than the chosen moieties at the particular sites, as well as binding of particular moieties at the other sites or regions.

In some configurations of a method or apparatus set forth herein, an assay reagent may recognize or bind an analyte that is a target analyte for the assay reagent. Nevertheless, the assay reagent may orthogonally bind to non-target materials or substances, such as non-target materials or substances present in an array that also includes the target analyte (e.g., anchoring moieties). Accordingly, orthogonal binding phenomena may be defined in certain cases as “analyte orthogonal binding interactions” or “non-analyte orthogonal binding interactions.” An analyte orthogonal binding interaction may refer to a binding interaction between a moiety and a non-analyte component of a single-analyte system, in which the moiety is expected or intended to form a binding interaction with an analyte of a single-analyte system. For example, an analyte orthogonal binding interaction may comprise an affinity agent becoming bound to an interstitial region of a single-analyte array. A non-analyte binding interaction may refer to a binding interaction between a moiety and a component of a single-analyte system (including an analyte), in which the moiety is not expected or intended to form a binding interaction with the component of a single-analyte system. For example, a blocking moiety (e.g., albumin, dextran, etc.) that is configured to remain unbound in solution may become bound to a surface-coupled molecule adjacent an analyte on a single-analyte array, thereby sterically impeding access to the analyte.

Systems or methods set forth herein may utilize one or more fluidic media to implement a process or step thereof. For array-based processes and systems, fluidic media may be provided for various process steps, including preparing arrays, attaching analytes to arrays, associating affinity agents to analytes, dissociating affinity agents from analytes, rinsing unbound moieties from array surfaces, performing detection processes on arrays, displacing a fluidic medium from contact with an array or other system components, and various other chemical and/or physical alterations of analytes or array components. A fluidic medium may be formulated to deliver a plurality of macromolecules (e.g., analytes, affinity agents) to an array as set forth herein. A fluidic medium may be formulated to mediate an interaction between macromolecules (e.g., an interaction between an analyte and an affinity agent).

A fluidic medium may be a single-phase or multi-phase fluidic medium. A multi-phase fluidic medium can include a gas phase and a liquid phase or at least two immiscible liquids. A multi-phase fluidic medium may comprise an interface between a first phase and a second phase. An interface between two fluidic phases may be laminar (e.g., an oil phase floating on an aqueous phase) or dispersed (e.g., bubbles, vesicles or droplets). A dispersed interface may be formed by a process such as emulsification. A divided interface may be stable (e.g., an emulsion) or unstable (e.g., a flocculating suspension). A multi-phase fluidic medium may comprise a colloidal agent that mediates an interface between a first phase and a second phase.

A fluidic medium can further contain solids, including particles (e.g., microparticles, nanoparticles). A fluidic medium comprising solids may be provided as a mixture, a suspension, or a slurry. It may be advantageous to provide a fluidic medium comprising a mixture or suspension of macromolecules. In some cases, solubility or suspendability of solids, such as particles or macromolecules, within a fluidic medium can be modulated by the composition of the fluidic medium. For example, alteration of fluidic properties such as solvent composition, ionic strength, and/or pH can induce precipitation, sedimentation, or flocculation of solvated or suspended solids.

A fluidic medium may be formulated with any one of numerous components depending upon its intended application. A fluidic medium can comprise one or more solvents. A single-phase fluidic medium can comprise two or more miscible solvents. In a mixture of miscible solvents, a solvent may be considered a base solvent if it comprises a greater than 50% fraction on a mass, molar, or volumetric basis. A miscible solvent may be mixed into a base solvent to alter a physical property of the base solvent, such as polarity, density, pH, viscosity, or surface tension. A fluidic medium can comprise a polar solvent or a non-polar solvent. A fluidic medium can comprise a protic or aprotic solvent. A fluidic medium can comprise an aqueous medium. A fluidic medium can comprise an organic solvent, such as acetic acid, acetone, acetonitrile, benzene, a butanol, 2-butanone, carbon tetrachloride, chlorobenzene, chloroform, cyclohexane, 1,2-dichloroethane, diethylene glycol, diethyl ether, diglyme, 1,2-dimethoxy-ethane, dimethylformamide, dimethyl sulfoxide, 1,4-dioxane, ethanol, ethyl acetate, ethylene glycol, glycerin, heptane, hexamethylphosphoramide, hexamethylphophorus triamide, hexanes, methanol, methyl t-butyl ether, methylene chloride, N-methyl-pyrrolidinone, nitromethane, pentane, petroleum ether, 1-proponal, 2-propanol, pyridine, tetrahydrofuran, toluene, triethyl amine, xylene, or a combination thereof. A fluidic medium can comprise a polar solvent, such as N-methyl pyrrolidone, tetrahydrofuran, ethyl acetate, acetone, dimethylfuran, acetonitrile, dimethyl sulfoxide, propylene carbonate, N-butanol, isopropyl alcohol, nitromethane, ethanol, methanol, acetic acid, or a combination thereof. A fluidic medium can comprise a non-polar solvent, such as benzene, carbon tetrachloride, chloroform, cyclohexane, dichloromethane, dimethoxyethane, ethyl ether, heptane, hexachloroethane, hexane, limonene, naphtha, pentane, tetrachloroethylene, tetrahydrofuran, toluene, xylenes, and combinations thereof. In some cases, a fluidic medium may comprise an aprotic solvent, such as N-methyl pyrrolidone, tetrahydrofuran, ethyl acetate, acetone, dimethylfuran, acetonitrile, dimethyl sulfoxide, propylene carbonate, or a combination thereof.

A fluidic medium may further comprise one or more components, including: 1) an ionic species, 2) a buffering agent, 3) a surfactant or detergent, 4) a chelating agent, 5) a denaturing agent or a chaotrope, 6) a cosmotropic or crowding agent, 7) a clouding agent, 8) a reactive scavenger, and 9) a blocking agent.

A fluidic medium may comprise one or more ionic species. An ionic species may be provided to a fluidic medium as a salt, thereby providing an anionic species and a cationic species to the fluidic medium. An ionic species can include a zwitterionic species. A fluidic medium may comprise a cationic species such as Na⁺, K⁺, Ag⁺, Cu⁺, NH⁴⁺, Mg²⁺, Ca²⁺, Cu²⁺, Cd²⁺, Zn²⁺, Fe²⁺, Co²⁺, Ni²⁺, Cr²⁺, Mn²⁺, Ge²⁺, Sn²⁺, Al³⁺, Cr³⁺, Fe³⁺, Co³⁺, Ni³⁺, Ti³⁺, Mn³⁺, Si⁴⁺, V⁴⁺, Ti⁴⁺, Mn⁴⁺, Ge⁴⁺, Se⁴⁺, V⁵⁺, Mn⁵⁺, Mn⁶⁺, Se⁶⁺, and combinations thereof. A fluidic medium may comprise an anionic species such as F⁻, Cl⁻, Br⁻, ClO₃⁻, H₂PO₄⁻, HCO₃⁻, HSO₄⁻, OH⁻, I⁻, NO₃⁻, NO₂⁻, MnO₄⁻, SCN⁻, CO₃²⁻, CrO₄²⁻, Cr₂O₇²⁻, HPO₄²⁻; SO₄²⁻, SO₃²⁻, PO₄³⁻, and combinations thereof. A fluidic medium may comprise a chelating agent, such as ethylenediaminetetraacetic acid (EDTA), nitrilotriacetic acid, n-hydroxyethylenediaminetetraacetic acid (HEDTA), oxalic acid, malic, acid, rubeanic acid, citric acid, or combinations thereof.

A fluidic medium may include a buffering species including, but not limited to, MES, Tris, Bis-tris, Bis-tris propane, ADA, ACES, PIPES, MOPSO, MOPS, BES, TES, HEPES, HEPBS, HEPPSO, DIPSO, MOBS, TAPSO, TAPS, TABS, POPSO, TEA, EPPS, Tricine, Gly-Gly, Bicine, AMPD, AMPSO, AMP, CHES, CAPSO, CAPS, PBS, and CABS.

A fluidic medium may comprise a surfactant or detergent. A surfactant or detergent may comprise a cationic surfactant or detergent, an anionic surfactant or detergent, a zwitterionic surfactant or detergent, an amphoteric surfactant or detergent, or a non-ionic surfactant or detergent. A fluidic medium may include a surfactant species including, but not limited to, stearic acid, lauric acid, oleic acid, sodium dodecyl sulfate, sodium dodecyl benzene sulfonate, dodecylamine hydrochloride, hexadecyltrimethylammonium bromide, polyethylene oxide, nonylphenyl ethoxylates, Triton X, pentapropylene glycol monododecyl ether, octapropylene glycol monododecyl ether, pentaethylene glycol monododecyl ether, octaethylene glycol monododecyl ether, lauramide monoethylamine, lauramide diethylamine, octyl glucoside, decyl glucoside, lauryl glucoside, Tween 20, Tween 80, n-dodecyl-β-D-maltoside, nonoxynol 9, glycerol monolaurate, polyethoxylated tallow amine, poloxamer, digitonin, zonyl FSO, 2,5-dimethyl-3-hexyne-2,5-diol, Igepal CA630, Aerosol-OT, triethylamine hydrochloride, cetrimonium bromide, benzethonium chloride, octenidine dihydrochloride, cetylpyridinium chloride, adogen, dimethyldioctadecylammonium chloride, CHAPS, CHAPSO, cocamidopropyl betaine, amidosulfobetaine-16, lauryl-N,N-(dimethylammonio)butyrate, lauryl-N,N-(dimethyl)-glycinebetaine, hexadecyl phosphocholine, lauryldimethylamine N-oxide, lauryl-N,N-(dimethyl)-propanesulfonate, 3-(1-pyridinio)-1-propanesulfonate, 3-(4-tert-butyl-1-pyridinio)-1-propanesulfonate, N-laurylsarcosine, and combinations thereof.

A fluidic medium may comprise a denaturing or chaotropic species, such as acetic acid, trichloroacetic acid, sulfosalicylic acid, sodium bicarbonate, ethanol, ethylenediamine tetraacetic acid (EDTA), urea, guanidinium chloride, lithium perchlorate, sodium dodecyl sulfate, 2-mercaptoethanol, dithiothreitol, tris(2-carboxyethyl) phosphine (TCEP), or a combination thereof. A denaturing or chaotropic species may be provided to alter a conformational state of an array component (e.g., causing denaturation of a polypeptide), or may be provided to maintain a conformational state of an array component (e.g., maintaining a polypeptide in a denatured or partially-denatured state).

A fluidic medium may comprise a cosmotropic species, such as carbonate ion, sulfate ion, phosphate ion, magnesium ion, lithium ion, zinc ion, aluminum ion, trehalose, glucose, proline, tert-butanol, or a combination thereof. A fluidic medium may comprise a clouding agent such as sodium chloride, potassium chloride, sodium bromide, potassium bromide, sodium nitrate, sodium sulfate, sodium phosphate, or a combination thereof. A cosmotropic species may be provided to decrease a separation distance between molecules and array components (e.g., causing smaller separation between an affinity agent and an analyte).

A fluidic medium may comprise a reactive scavenger species. A reactive scavenger may be provided to reduce solution-phase concentrations of reactive species (e.g., oxidizing or reducing species). A reactive scavenger may be provided during a photon-mediated process (e.g., fluorescent imaging) to reduce photodamage or other deleterious photon-related processes (e.g., singlet oxygen generation, free radical generation). Exemplary reactive scavenger species can include ascorbic acid, 9,10-anthracenediyl-bis(methylene)dimalonic acid (ABDA), epigallocatechin gallate (EPGG), N-acetyl-L-cysteine, caffeic acid, reseveratrol, 4-hydroxy-2,2,6,6-tetramethylpiperidin-1-oxyl (TEMPOL), sodium sulfite, 1,4-diazabicyclo[2.2.2]octane (DABCO), sodium pyruvate, N,N′-dimethylthiourea (DMTU), mannitol, dimethyl sulfoxide (DMSO), 6-hydroxy-2,5,7,8-tetramethylchroman-2-carboxylic acid (Trolox), 2-phenyl-1,2-benzisoselenazol-3(2H)-one (Ebselen), α-tocopherol, uric acid, sodium azide, manganese(III)-tetrakis(4-benzoic acid) porphyrin, 4,5-dihydroxybenzene-1,3-disulfonate, or a combination thereof. Other useful reactive scavengers and methods for their use in reducing photodamage or other deleterious photon-related processes are set forth in U.S. Pat. No. 10,106,851, which is incorporated herein by reference.

A fluidic medium may comprise a blocking agent. A blocking agent may include any species that inhibits orthogonal binding phenomena between assay agents and array components, such as polyethylene glycol, dextrans, albumin, or synthetic polymers such as PF-127 or polyvinylpyrrolidone.

A method set forth herein may involve a step of delivering a fluidic medium to a vessel (e.g., a flow cell, a fluidic cartridge, a reactor or microreactor, etc.) containing an array, as set forth herein. In some cases, after delivering a fluidic medium to a vessel, the fluidic medium may be incubated with an array within the vessel. Incubation of a fluidic medium with an array may be substantially quiescent. Alternatively, incubation of a fluidic medium with an array may be non-quiescent due to mixing, agitation, or circulation of the fluidic medium within or through the vessel.

A method set forth herein may involve a step of altering a fluidic medium with respect to one or more properties of the fluidic medium. Altered properties can include temperature, pH, ionic strength, and composition of the fluidic medium. In some cases, altering a fluidic medium may comprise displacing a first fluidic medium having a first property (e.g., temperature, pH, ionic strength, composition) with a second fluidic medium having a second property, in which the first property differs from the second property. In other cases, altering a fluidic medium may comprise mixing a second fluidic medium or chemical component (e.g., a solute) into a first fluidic medium. For example, a pH of a fluidic medium may be altered by adding an acid or base species to a fluidic medium in a vessel. In another example, a fluidic medium may be diluted or condensed with respect to ionic strength or concentration of a component by addition of a second fluidic medium to the vessel.

A fluidic medium may be provided at, heated to, cooled to, or maintained at a temperature of at least about −80 degrees Celsius (C), −50° C., −10° C., −5° C., 0° C., 5° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 35° C., 40° C., 45° C., 50° C., 60° C., 70° C., 80° C., 90° C., 95° C., or more than 95° C. Alternatively or additionally, a fluidic medium may be provided at, heated to, cooled to, or maintained at a temperature of no more than about 95° C., 90° C., 80° C., 70° C., 60° C., 50° C., 45° C., 40° C., 35° C., 30° C., 29° C., 28° C., 27° C., 26° C., 25° C., 24° C., 23° C., 22° C., 21° C., 20° C., 19° C., 18° C., 17° C., 16° C., 15° C., 14° C., 13° C., 12° C., 11° C., 10° C., 5° C., 0° C., −5° C., −10° C., −50° C., −80° C., or less than −80° C.

A fluidic medium may be provided at or adjusted to a pH of at least about 0.0, 0.5, 1.0, 1.5, 2.0, 2.5, 3.0, 3.5, 4.0, 4.5, 5.0, 5.5, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.5, 9.0, 9.5, 10.0, 10.5, 11.0, 11.5, 12.0, 12.5, 13.0, 13.5, 14.0, or more than 14.0. Alternatively or additionally, a fluidic medium may be provided at or adjusted to a pH of no more than about 14.0, 13.5, 13.0, 12.5, 12.0, 11.5, 11.0, 10.5, 10.0, 9.5, 9.0, 8.5, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.5, 5.0, 4.5, 4.0, 3.5, 3.0, 2.5, 2.0, 1.5, 1.0, 0.5, or less than 0.5

A component of a fluidic medium may be provided at or adjusted to a molar concentration of at least about 0.0001 moles per liter (M), 0.001M, 0.01M, 0.02M, 0.03M, 0.04M, 0.05M, 0.06M, 0.07M, 0.08M, 0.09M, 0.1M, 0.2M, 0.3M, 0.4M, 0.5M, 0.6M, 0.7M, 0.8M, 0.9M, 1M, 1.1M, 1.2M, 1.3M, 1.4M, 1.5M, 1.6M, 1.7M, 1.8M, 1.9M, 2M, 2.1M, 2.2M, 2.3M, 2.4M, 2.5M, 2.6M, 2.7M, 2.8M, 2.9M, 3M, 3.1M, 3.2M, 3.3M, 3.4M, 3.5M, 3.6M, 3.7M, 3.8M, 3.9M, 4M, 4.1M, 4.2M, 4.3M, 4.4M, 4.5M, 4.6M, 4.7M, 4.8M, 4.9M, 5M, 5.1M, 5.2M, 5.3M, 5.4M, 5.5M, 5.6M, 5.7M, 5.8M, 5.9M, 6M, 7M, 8M, 9M or more than 10M. Alternatively or additionally, a component of a fluidic medium may be provided at or adjusted to a molar concentration of no more than about 10 M, 9M, 8M, 7M, 6M, 5.9M, 5.8M, 5.7M, 5.6M, 5.5M, 5.4M, 5.3M, 5.2M, 5.1M, 5.0M, 4.9M, 4.8M, 4.7M, 4.6M, 4.5M, 4.4M, 4.3M, 4.2M, 4.1M, 4.0M, 3.9M, 3.8M, 3.7M, 3.6M, 3.5M, 3.4M, 3.3M, 3.2M, 3.1M, 3.0M, 2.9M, 2.8M, 2.7M, 2.6M, 2.5M, 2.4M, 2.3M, 2.2M, 2.1M, 2.0M, 1.9M, 1.8M, 1.7M, 1.6M, 1.5M, 1.4M, 1.3M, 1.2M, 1.1M, 1.0M, 0.9M, 0.8M, 0.7M, 0.6M, 0.5M, 0.4M, 0.3M, 0.2M, 0.1M, 0.09M, 0.08M, 0.07M, 0.06M, 0.05M, 0.04M, 0.03M, 0.02M, 0.01M, 0.001M, 0.001M, or less than about 0.001M.

A component of a fluidic medium may be provided at or adjusted to a weight or volumetric percentage of at least about 0.0001%, 0.001%, 0.005%, 0.01%, 0.05%, 0.1%, 0.5%, 1%, 2%, 3%, 4%, 45%, 6%, 7%, 8%, 9%, 10%, 20%, 30%, 40%, 50%, or more than 50%. Alternatively or additionally, a component of a fluidic medium may be provided at or adjusted to a weight or volumetric percentage of no more than about 50%, 40%, 30%, 20%, 10%, 9%, 8%, 7%, 6%, 5%, 4%, 3%, 2%, 1%, 0.5%, 0.1%, 0.05%, 0.01%, 0.005%, 0.001%, 0.0001%, or less than 0.0001%.

The methods, compositions and apparatus of the present disclosure are particularly well suited for use with proteins. Although proteins are exemplified throughout the present disclosure, it will be understood that other analytes can be similarly used. Exemplary analytes include, but are not limited to, biomolecules, polysaccharides, nucleic acids, lipids, metabolites, hormones, vitamins, enzyme cofactors, therapeutic agents, candidate therapeutic agents or combinations thereof. An analyte can be a non-biological atom or molecule, such as a synthetic polymer, metal, metal oxide, ceramic, semiconductor, mineral, or a combination thereof.

One or more proteins that are used in a method, composition or apparatus herein, can be derived from a natural or synthetic source. Exemplary sources include, but are not limited to biological tissues, fluids, cells or subcellular compartments (e.g. organelles). For example, a sample can be derived from a tissue biopsy, biological fluid (e.g. blood, sweat, tears, plasma, extracellular fluid, urine, mucus, saliva, semen, vaginal fluid, synovial fluid, lymph, cerebrospinal fluid, peritoneal fluid, pleural fluid, amniotic fluid, intracellular fluid, extracellular fluid, etc.), fecal sample, hair sample, cultured cell, culture media, fixed tissue sample (e.g. fresh frozen or formalin-fixed paraffin-embedded) or product of a protein synthesis reaction. A protein source may include any sample where a protein is a native or expected constituent. For example, a primary source for a cancer biomarker protein may be a tumor biopsy sample or bodily fluid. Other sources include environmental samples or forensic samples.

Exemplary organisms from which proteins or other analytes can be derived include, for example, a mammal such as a rodent, mouse, rat, rabbit, guinea pig, ungulate, horse, sheep, pig, goat, cow, cat, dog, primate, non-human primate or human; a plant such as Arabidopsis thaliana, tobacco, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a dictyostelium discoideum; a fungi such as Pneumocystis carinii, Takifugu rubripes, yeast, Saccharamoyces cerevisiae or Schizosaccharomyces pombe; or a Plasmodium falciparum. Proteins can also be derived from a prokaryote such as a bacterium, Escherichia coli, staphylococci or Mycoplasma pneumoniae; an archae; a virus such as Hepatitis C virus, influenza virus, coronavirus, or human immunodeficiency virus; or a viroid. Proteins can be derived from a homogeneous culture or population of the above organisms or alternatively from a collection of several different organisms, for example, in a community or ecosystem.

In some cases, a protein or other biomolecule can be derived from an organism that is collected from a host organism. For example, a protein may be derived from a parasitic, pathogenic, symbiotic, or latent organism collected from a host organism. A protein can be derived from an organism, tissue, cell or biological fluid that is known or suspected of being linked with a disease state or disorder (e.g., cancer). Alternatively, a protein can be derived from an organism, tissue, cell or biological fluid that is known or suspected of not being linked to a particular disease state or disorder. For example, the proteins isolated from such a source can be used as a control for comparison to results acquired from a source that is known or suspected of being linked to the particular disease state or disorder. A sample may include a microbiome or substantial portion of a microbiome. In some cases, one or more proteins used in a method, composition or apparatus set forth herein may be obtained from a single source and no more than the single source. The single source can be, for example, a single organism (e.g. an individual human), single tissue, single cell, single organelle (e.g. endoplasmic reticulum, Golgi apparatus or nucleus), or single protein-containing particle (e.g., a viral particle or vesicle).

A method, composition or apparatus of the present disclosure can use or include a plurality of proteins having any of a variety of compositions such as a plurality of proteins composed of a proteome or fraction thereof. For example, a plurality of proteins can include solution-phase proteins, such as proteins in a biological sample or fraction thereof, or a plurality of proteins can include proteins that are immobilized, such as proteins attached to a particle or solid support. By way of further example, a plurality of proteins can include proteins that are detected, analyzed or identified in connection with a method, composition or apparatus of the present disclosure. The content of a plurality of proteins can be understood according to any of a variety of characteristics such as those set forth below or elsewhere herein.

A plurality of proteins can be characterized in terms of total protein mass. The total mass of protein in a liter of plasma has been estimated to be 70 g and the total mass of protein in a human cell has been estimated to be between 100 pg and 500 pg depending upon cells type. See Wisniewski et al. Molecular & Cellular Proteomics 13:10.1074/mcp.M113.037309, 3497-3506 (2014), which is incorporated herein by reference. A plurality of proteins used or included in a method, composition or apparatus set forth herein can include at least 1 pg, 10 pg, 100 pg, 1 ng, 10 ng, 100 ng, 1 mg, 10 mg, 100 mg, 1 mg, 10 mg, 100 mg or more protein by mass. Alternatively or additionally, a plurality of proteins may contain at most 100 mg, 10 mg, 1 mg, 100 mg, 10 mg, 1 mg, 100 ng, 10 ng, 1 ng, 100 pg, 10 pg, 1 pg or less protein by mass.

A plurality of proteins can be characterized in terms of percent mass relative to a given source such as a biological source (e.g. cell, tissue, or biological fluid such as blood). For example, a plurality of proteins may contain at least 60%, 75%, 90%, 95%, 99%, 99.9% or more of the total protein mass present in the source from which the plurality of proteins was derived. Alternatively or additionally, a plurality of proteins may contain at most 99.9%, 99%, 95%, 90%, 75%, 60% or less of the total protein mass present in the source from which the plurality of proteins was derived.

A plurality of proteins can be characterized in terms of total number of protein molecules. The total number of protein molecules in a Saccharomyces cerevisiae cell has been estimated to be about 42 million protein molecules. See Ho et al., Cell Systems (2018), DOI: 10.1016/j.cels.2017.12.004, which is incorporated herein by reference. A plurality of proteins used or included in a method, composition or apparatus set forth herein can include at least 1 protein molecule, 10 protein molecules, 100 protein molecules, 1×10⁴ protein molecules, 1×10⁶ protein molecules, 1×10⁸ protein molecules, 1×10¹⁰ protein molecules, 1 mole (6.02214076×10²³ molecules) of protein, 10 moles of protein molecules, 100 moles of protein molecules or more. Alternatively or additionally, a plurality of proteins may contain at most 100 moles of protein molecules, 10 moles of protein molecules, 1 mole of protein molecules, 1×10¹⁰ protein molecules, 1×10⁸ protein molecules, 1×10⁶ protein molecules, 1×10⁴ protein molecules, 100 protein molecules, 10 protein molecules, 1 protein molecule or less.

A plurality of proteins can be characterized in terms of the variety of full-length primary protein structures in the plurality. For example, the variety of full-length primary protein structures in a plurality of proteins can be equated with the number of different protein-encoding genes in the source for the plurality of proteins. Whether or not the proteins are derived from a known genome or from any genome at all, the variety of full-length primary protein structures can be counted independent of presence or absence of post translational modifications in the proteins. A human proteome is estimated to have about 20,000 different protein-encoding genes such that a plurality of proteins derived from a human can include up to about 20,000 different primary protein structures. See Aebersold et al., Nat. Chem. Biol. 14:206-214 (2018), which is incorporated herein by reference. Other genomes and proteomes in nature are known to be larger or smaller. A plurality of proteins used or included in a method, composition or apparatus set forth herein can have a complexity of at least 2, 5, 10, 100, 1×103, 1×10⁴, 2×10⁴, 3×10⁴ or more different full-length primary protein structures. Alternatively or additionally, a plurality of proteins can have a complexity that is at most 3×10⁴, 2×10⁴, 1×10⁴, 1×103, 100, 10, 5, 2 or fewer different full-length primary protein structures.

In relative terms, a plurality of proteins used or included in a method, composition or apparatus set forth herein may contain at least one representative for at least 60%, 75%, 90%, 95%, 99%, 99.9% or more of the proteins encoded by the genome of a source from which the sample was derived. Alternatively or additionally, a plurality of proteins may contain a representative for at most 99.9%, 99%, 95%, 90%, 75%, 60% or less of the proteins encoded by the genome of a source from which the sample was derived.

A plurality of proteins can be characterized in terms of the variety of primary protein structures in the plurality including transcribed splice variants. The human proteome has been estimated to include about 70,000 different primary protein structures when splice variants ae included. See Aebersold et al., Nat. Chem. Biol. 14:206-214 (2018), which is incorporated herein by reference. Moreover, the number of the partial-length primary protein structures can increase due to fragmentation that occurs in a sample. A plurality of proteins used or included in a method, composition or apparatus set forth herein can have a complexity of at least 2, 5, 10, 100, 1×10³, 1×10⁴, 7×10⁴, 1×10⁵, 1×10⁶ or more different primary protein structures. Alternatively or additionally, a plurality of proteins can have a complexity that is at most 1×10⁶, 1×10⁵, 7×10⁴, 1×10⁴, 1×10³, 100, 10, 5, 2 or fewer different primary protein structures.

A plurality of proteins can be characterized in terms of the variety of protein structures in the plurality including different primary structures and different proteoforms among the primary structures. Different molecular forms of proteins expressed from a given gene are considered to be different proteoforms. Proteoforms can differ, for example, due to differences in primary structure (e.g. shorter or longer amino acid sequences), different arrangement of domains (e.g. transcriptional splice variants), or different post translational modifications (e.g. presence or absence of phosphoryl, glycosyl, acetyl, or ubiquitin moieties). The human proteome is estimated to include hundreds of thousands of proteins when counting the different primary structures and proteoforms. See Aebersold et al., Nat. Chem. Biol. 14:206-214 (2018), which is incorporated herein by reference. A plurality of proteins used or included in a method, composition or apparatus set forth herein can have a complexity of at least 2, 5, 10, 100, 1×10³, 1×10⁴, 1×10⁵, 1×10⁶, 5×10⁶, 1×10⁷ or more different protein structures. Alternatively or additionally, a plurality of proteins can have a complexity that is at most 1×10⁷, 5×10⁶, 1×10⁶, 1×10⁵, 1×10⁴, 1×10³, 100, 10, 5, 2 or fewer different protein structures.

A plurality of proteins can be characterized in terms of the dynamic range for the different protein structures in the sample. The dynamic range can be a measure of the range of abundance for all different protein structures in a plurality of proteins, the range of abundance for all different primary protein structures in a plurality of proteins, the range of abundance for all different full-length primary protein structures in a plurality of proteins, the range of abundance for all different full-length gene products in a plurality of proteins, the range of abundance for all different proteoforms expressed from a given gene, or the range of abundance for any other set of different proteins set forth herein. The dynamic range for all proteins in human plasma is estimated to span more than 10 orders of magnitude from albumin, the most abundant protein, to the rarest proteins that have been measured clinically. See Anderson and Anderson Mol Cell Proteomics 1:845-67 (2002), which is incorporated herein by reference. The dynamic range for plurality of proteins set forth herein can be a factor of at least 10, 100, 1×10³, 1×10⁴, 1×10⁶, 1×10⁸, 1×10¹⁰, or more. Alternatively or additionally, the dynamic range for plurality of proteins set forth herein can be a factor of at most 1×10¹⁰, 1×10⁸, 1×10⁶, 1×10⁴, 1×10³, 100, 10 or less.

The present disclosure provides compositions, apparatus and methods that are useful for detecting, characterizing and identifying proteoforms. For example, the presence or absence of a particular post-translational modification or a particular post-translationally modified amino acid can be determined. In some embodiments, a proteoform can be characterized with respect to the location(s) of one or more post-translational modifications in the amino acid sequence of the proteoform. Locations can be identified, for example, at a specific position of the amino acid sequence for the proteoform. However, in some cases, the location of a post-translational modification in a proteoform can be determined relative to a particular structural motif of the proteoform. For example, a post-translational moiety of a proteoform can be located relative to a short sequence of amino acids in the proteoform or relative to another post-translational moiety in the proteoform.

Methods of the present disclosure are particularly well suited for manipulating and detecting proteoforms. The presence or absence of post-translational modifications (PTM) can be detected using a composition, apparatus or method set forth herein. A PTM can be detected using an affinity agent that recognizes the PTM or based on a chemical property of the PTM. In some configurations, methods set forth herein can be used to differentially manipulate proteoforms based on unique molecular properties or to distinguish one proteoform from another.

A post-translational modification may be one or more of myristoylation, palmitoylation, isoprenylation, prenylation, farnesylation, geranylgeranylation, lipoylation, flavin moiety attachment, Heme C attachment, phosphopantetheinylation, retinylidene Schiff base formation, dipthamide formation, ethanolamine phosphoglycerol attachment, hypusine, beta-Lysine addition, acylation, acetylation, deacetylation, formylation, alkylation, methylation, C-terminal amidation, arginylation, polyglutamylation, polyglycylation, butyrylation, gamma-carboxylation, glycosylation, glycation, polysialylation, malonylation, hydroxylation, iodination, nucleotide addition, phosphoate ester formation, phosphoramidate formation, phosphorylation, adenylylation, uridylylation, propionylation, pyrolglutamate formation, S-glutathionylation, S-nitrosylation, S-sulfenylation, S-sulfinylation, S-sulfonylation, succinylation, sulfation, glycation, carbamylation, carbonylation, isopeptide bond formation, biotinylation, carbamylation, oxidation, reduction, pegylation, ISGylation, SUMOylation, ubiquitination, neddylation, pupylation, citrullination, deamidation, elminylation, disulfide bridge formation, isoaspartate formation, and racemization. Proteoforms can differ with regard to presence or absence of a post-translational modification, type of post-translational modification present, location of a post-translational modification, number of post-translational modifications present or combination thereof.

A post-translational modification may occur at a particular type of amino acid residue in a protein. For example, the phosphate moiety of a particular proteoform can be present on a serine, threonine, tyrosine, histidine, cysteine, lysine, aspartate or glutamate residue. In another example, an acetyl moiety of a particular proteoform can be present on the N-terminus or on a lysine of a protein. In another example, a serine or threonine residue of a proteoform can have an O-linked glycosyl moiety, or an asparagine residue of a proteoform can have an N-linked glycosyl moiety. In another example, a proline, lysine, asparagine, aspartate or histidine amino acid of a proteoform can be hydroxylated. In another example, a proteoform can be methylated at an arginine or lysine amino acid. In another example, a proteoform can be ubiquitinated at the N-terminal methionine or at a lysine amino acid.

A post-translationally modified version of a given amino acid can include a post-translational moiety at a side chain position that is unmodified in a standard version of the amino acid. Post-translationally modified lysines can include epsilon amines attached to post-translational moieties, whereas standard lysines have epsilon amines lacking the post-translational moieties. Post-translationally modified histidines can include side-chain tertiary amines attached to post-translational moieties, whereas in standard histidines the side-chain amines are secondary amines lacking the post-translational moieties. Post-translationally modified versions of aspartates or glutamates can include side-chain carbonyls, esters or amides attached to post-translational moieties, whereas in standard versions of aspartates or glutamates the side-chains have carboxyls lacking the post-translational moieties. Post-translationally modified versions of arginines can include side-chain amines attached to post-translational moieties, whereas in standard versions of arginines the side-chain amines lack the post-translational moieties. Post-translationally modified versions of cysteines can include thioethers attached to post-translational moieties, whereas standard versions of cysteines have sulfurs lacking the post-translational moieties. Post-translationally modified versions of serines, threonines or tyrosines can include ethers or esters attached to post-translational moieties, whereas standard versions of serines, threonines or tyrosines have hydroxyls lacking the post-translational moieties.

A method of the present disclosure can include a step of removing post-translational moieties from post-translationally modified amino acids, thereby forming standard amino acids. In some cases, an enzyme can be used to remove a post-translational moiety from an amino acid. An enzyme that removes a post-translational moiety independently of amino acid sequence context surrounding the post-translationally modified amino acid can be used. In other cases, a sequence-specific enzyme can be used to remove a post-translational moiety.

A phosphatase enzyme can be used to remove a phosphate moiety from an amino acid. A broadscale (e.g. sequence agnostic) phosphatase such as alkaline phosphatase can be useful. Protein phosphatases are available for removing phosphate moieties from various types of amino acids. Exemplary protein phosphatases include, but are not limited to, tyrosine-specific kinases such as PTP1B; serine/threonine-specific phosphatases such as PP2C and PPP2CA; dual specificity phosphatases such as lambda protein phosphatase or VHR, both of which can remove phosphate moieties from serine, threonine or tyrosine residues; or histidine phosphatase such as PHP. Phosphatases or kinases that are specific to particular signal transduction pathways can be used to remove phosphates in a sequence specific manner if desired.

Several enzymes are available for removing post-translational moieties from lysines. Examples are set forth in Wang and Cole, Cell Chemical Biology 27:953-969 (2020) (which is incorporated herein by reference) and below. Lysine deacetylases can be used to remove acetyl moieties from lysines. For example, at least eighteen different protein lysine deacetylases (e.g. histone deacetylases) are known to remove acetyl moieties from lysines in human proteins. Lysine demethylases can be used to remove methyl moieties from lysines. Deubiquitinases (DUBs) are isopeptidases that sever the amide bond between a lysine side chain of a protein and the ubiquitin (Ub) C terminus. Many DUBs can cleave Ub-Ub amide linkages whereas others show selectivity for particular ubiquitinated proteins.

Optionally, glycan moieties can be released from proteins in a method of the present disclosure. For example, N-glycans or O-glycans can be released from glycoproteins using glycosidases. Any of a variety of enzymes can be used to remove glycans from proteins. For example, α-2-3,6,8,9-Neuraminidase can be used to cleave non-reducing terminal branched and unbranched sialic acids; β-1,4-galactosidase can be used to remove β-1,4-linked nonreducing terminal galactose from proteins; β-N-acetylgucosaminidase can be used to cleave non-reducing terminal β-linked N-acetylgucosamine from proteins; endo-a-N-acetylgalactosaminidase can be used to remove O-glycosylation, for example, removing serine- or threonine-linked unsubstituted Galb 1,3GalNac; and PNGase F can be used to cleave oligosaccharides from asparagines. Exemplary reagents and methods for releasing glycans from proteins are set forth in Zhang et al. Frontiers in Chemistry, vol 8, Article 508 (2020) doi: 10.3389/fchem.2020.00508, which is incorporated herein by reference.

A plurality of extant proteins may contain two or more proteoforms of a single species of protein (e.g., at least 2, 3, 4, 5, 10, 20, 50, 100, or more than 100 proteoforms). Alternatively, a plurality of extant proteins may contain only a single proteoform of a single species. A plurality of extant proteins may contain at least one species of protein having two or more proteoforms (e.g., at least 2, 10, 50, 100, 500, 1000, 5000, 10000, or more than 10000 species of protein having two or more proteoforms). Alternatively, a plurality of extant proteins may contain at least one species of protein having only one proteoform (e.g., at least 2, 10, 50, 100, 500, 1000, 5000, 10000, or more than 10000 species of protein having only one proteoform).

A method of identifying extant proteins may further include identifying proteoforms of extant proteins. Accordingly, a method of identifying a proteoform of an individual protein can include the steps of: i) identifying a primary amino acid sequence of the protein based upon a binding profile of the protein, thereby identifying the protein, and ii) identifying a proteoform of the protein. Proteoform-specific affinity agents may be useful for identifying the proteoform of an extant protein. A proteoform-specific affinity agent can be a promiscuous affinity agent, for example binding to post-translational modifications (e.g., methylations, phosphorylations, glycosylations, etc.) of a plurality of protein species and/or proteoforms. A proteoform-specific affinity agent can be highly specific to a single proteoform of one or more protein species (e.g., only binding to a single post-translationally modified amino acid of a single protein species). A proteoform may be identified in part by detecting presence of binding of one or more affinity agents to an extant protein. Alternatively, a proteoform may be identified in part by an absence of detectable binding of one or more affinity agents to an extant protein (e.g., due to absence of a post-translational modification at an amino acid residue of the extant protein, due to absence of a bindable epitope due to splice variation of the extant protein, etc.).

In some cases, it may be preferable to contact extant proteins with a proteoform-specific affinity agent before contacting the extant proteins with other promiscuous or non-proteoform affinity agents. Presence of certain post-translational modification may inhibit binding of affinity agents to epitopes where said post-translational modification are present. Accordingly, a method may further comprise a step of removing post-translation modification (e.g., chemically or enzymatically) from extant proteins. After detecting binding of proteoform-specific affinity agents to extant proteins, and optionally removing one or more post-translational modification from the extant proteins, the extant proteins may be subsequently contacted with a series of promiscuous affinity agents, thereby providing binding profiles for each individual extant protein.

1. A composition, comprising:
a) a nucleic acid nanoparticle comprising a face, wherein the face contains a first attachment site and a second attachment site;
b) a first metal nanoparticle and a second metal nanoparticle, wherein the first metal nanoparticle is attached to the first attachment site and the second metal nanoparticle is attached to the second attachment site;
c) an entity coupled to the nucleic acid nanoparticle, wherein the entity is disposed between the first metal nanoparticle and the second metal nanoparticle; and
d) a pendant single-stranded nucleic acid attached to the nucleic acid nanoparticle.

2. The composition of claim 1, wherein the first metal nanoparticle comprises a metal selected from the group consisting of rhenium, ruthenium, rhodium, palladium, silver, osmium, iridium, platinum, gold, copper, and combinations thereof.

3. The composition of claim 1, wherein the second metal nanoparticle comprises a metal selected from the group consisting of rhenium, ruthenium, rhodium, palladium, silver, osmium, iridium, platinum, gold, copper, and combinations thereof.

4. The composition of claim 1, wherein the first metal nanoparticle has a same atomic composition as the second metal nanoparticle.

5. The composition of claim 1, wherein an atomic composition of the first metal nanoparticle differs from an atomic composition of the second metal nanoparticle.

6. The composition of claim 1, wherein a diameter of the first metal nanoparticle is substantially the same as a diameter of the second metal nanoparticle.

7. The composition of claim 1, wherein a diameter of the first metal nanoparticle is substantially the larger than a diameter of the second metal nanoparticle.

8.-11. (canceled)

12. The composition of claim 1, wherein the first metal nanoparticle and the second metal nanoparticle have a smallest separation gap of no more than about 20 nm.

13.-15. (canceled)

16. The composition of claim 1, wherein a complementary attachment moiety is attached to the first metal nanoparticle.

17. The composition of claim 16, wherein the complementary attachment moiety of the first metal nanoparticle is coupled to an attachment moiety of the first attachment site.

18.-29. (canceled)

30. The composition of claim 1, wherein the nucleic acid nanoparticle further comprises a second face, wherein the pendant single-stranded nucleic acid is attached to the second face.

31. The composition of claim 30, wherein the second face is substantially distal to the first face.

32.-46. (canceled)

47. The composition of claim 1, wherein the first attachment site or the second attachment site comprises a spacing moiety.

48.-51. (canceled)

52. A composition, comprising:
a) a solid support;
b) a nucleic acid nanoparticle attached to the solid support, wherein the nucleic acid nanoparticle comprises a face, wherein the face is substantially distal to the solid support, and wherein the face comprises a first attachment site, a second attachment site, and a third attachment site;
c) first metal nanoparticle and a second metal nanoparticle, wherein the first metal nanoparticle is attached to the first attachment site and the second metal nanoparticle is attached to the second attachment site; and
d) a polymeric chain coupled to the third attachment site, wherein the polymeric chain is disposed between the first metal nanoparticle and the second metal nanoparticle.

53. The composition of claim 52, further comprising a magnetic nanoparticle, wherein the magnetic nanoparticle is attached to the polymeric chain.

54. The composition of claim 52, wherein the polymeric chain comprises a secondary or tertiary structure.

55.-65. (canceled)

66. A method, comprising:
a) coupling a binding reagent to an analyte, wherein the analyte is immobilized on a solid support, and wherein the binding reagent comprises a nanoparticle cluster, wherein the nanoparticle cluster comprises a first metal nanoparticle, a second metal nanoparticle, and a fluorescent dye disposed between the first metal nanoparticle and the second metal nanoparticle;
b) contacting the nanoparticle cluster with light; and
c) detecting a fluorescent signal from the fluorescent dye, thereby identifying an address of the solid support containing the binding reagent coupled to the analyte.

67.-72. (canceled)

73. The method of claim 66, wherein the analyte is immobilized on the solid support at a site, wherein the solid support comprises an array of sites containing the site, and wherein individual sites of the plurality of sites each contain an immobilized analyte.

74. The method of claim 73, wherein contacting the nanoparticle cluster with light comprises rastering a light field across each site of the plurality of sites.

75. The method of claim 73, wherein contacting the nanoparticle cluster with light comprises simultaneously contacting a subset of the plurality of sites with a light field.

76.-95. (canceled)
